SEQUENCE LISTING (4 sequences)

SEQ ID NO: 1 - protein, 666 aa, Homo sapiens
  REGION 1..666: note = Human SEMA7A
MTPPPPGRAAPSAPRARVPGPPARLGLPLRLRLLLLLWAAAASAQGHLRSGPRIFAVWKGHVGQDRVDFGQTEPHTVLFHEPGSSSVWVGGRGKVYLFDFPEGKNASVRTVNIGSTKGSCLDKRDCENYITLLERRSEGLLACGTNARHPSCWNLVNGTVVPLGEMRGYAPFSPDENSLVLFEGDEVYSTIRKQEYNGKIPRFRRIRGESELYTSDTVMQNPQFIKATIVHQDQAYDDKIYYFFREDNPDKNPEAPLNVSRVAQLCRGDQGGESSLSVSKWNTFLKAMLVCSDAATNKNFNRLQDVFLLPDPSGQWRDTRVYGVFSNPWNYSAVCVYSLGDIDKVFRTSSLKGYHSSLPNPRPGKCLPDQQPIPTETFQVADRHPEVAQRVEPMGPLKTPLFHSKYHYQKVAVHRMQASHGETFHVLYLTTDRGTIHKVVEPGEQEHSFAFNIMEIQPFRRAAAIQTMSLDAERRKLYVSSQWEVSQVPLDLCEVYGGGCHGCLMSRDPYCGWDQGRCISIYSSERSVLQSINPAEPHKECPNPKPDKAPLQKVSLAPNSRYYLSCPMESRHATYSWRHKENVEQSCEPGHQSPNCILFIENLTAQQYGHYFCEAQEGSYFREAQHWQLLPEDGIMAEHLLGHACALAASLWLGVLPTLTLGLLVH

SEQ ID NO: 2 - protein, 10 aa, synthetic construct
  REGION 1..10: note = Synthetic Construct; 10mer peptide of human SEMA7A
ADRHPEVAQR

SEQ ID NO: 3 - protein, 13 aa, synthetic construct
  REGION 1..13: note = Synthetic Construct; 10mer peptide of human SEMA7A with linker for conjugation
CGGADRHPEVAQR

SEQ ID NO: 4 - protein, 41 aa, synthetic construct
  REGION 1..41: note = Synthetic Construct; 41mer peptide of human SEMA7A
QPIPTETFQVADRHPEVAQRVEPMGPLKTPLFHSKYHYQKV

(19) United States
(12) Patent Application Publication
(10) Pub. No.: US 2024/0174762 A1
(43) Pub. Date: May 30, 2024

(54) METHODS, COMPOSITIONS AND USES FOR TARGETING SEMA7A IN THE DIAGNOSIS AND TREATMENT OF HEALTH CONDITIONS

(71) Applicant: THE REGENTS OF THE UNIVERSITY OF COLORADO, A BODY CORPORATE, Denver, CO (US)

(72) Inventors: Traci R. LYONS, Denver, CO (US); Robert HODGES, Denver, CO (US)

(21) Appl. No.: 18/525,459

(22) Filed: Nov. 30, 2023

Related U.S. Application Data
(63) Continuation of application No. PCT/US22/31800, filed on Jun. 1, 2022.
(60) Provisional application No. 63/195,572, filed on Jun. 1, 2021.
(60) Provisional application No. 63/234,594, filed on Aug. 18, 2021.

(51) Int. Cl. C07K 16/28; A61K 39/00; A61P 35/00; C07K 14/47

(52) U.S. Cl. CPC C07K 16/2896; A61P 35/00; C07K 14/4703; A61K 2039/505

(57) ABSTRACT

Embodiments of the present invention generally relate to compositions and methods for targeting Semaphorin 7a (SEMA7A) in health conditions. In certain embodiments, compositions and methods disclosed herein concern using SEMA7A targeting for diagnosis and treatment of cancers, cardiac conditions, and other health conditions. In some embodiments, SEMA7A peptide fragments and fusion polypeptides can be used to generate antibodies of use to diagnose, reduce metastasis and treat conditions disclosed herein. In certain embodiments, SEMA7A levels can be determined to project progression of a condition. In some embodiments, cancers can be targeted. In other embodiments, cancer stem cells can be targeted. In certain embodiments, SEMA7A levels can be assessed in breast cancers. In other embodiments, monoclonal antibodies against SEMA7A polypeptides can be used to treat cancers such as breast cancer and other cancers having aberrant SEMA7A expression in a subject.

### PRIORITY

This U.S. Continuation application claims priority to International Application No. PCT/US2022/031800, filed Jun. 1, 2022, which claims priority under 35 U.S.C. § 119(e) to U.S. Provisional Application No. 63/195,572, filed Jun. 1, 2021 and U.S. Provisional Application No. 63/234,594, filed Aug. 18, 2021. These applications are incorporated by reference in their entirety for all purposes.

### GOVERNMENT FUNDING

This invention was made with government support under grant number RO1 CA21696-01A1 from the National Institutes of Health. The government has certain rights in the invention.

### FIELD

Embodiments of the present invention generally relate to compositions and methods for diagnosing and treating cancer and other health conditions. In certain embodiments, compositions and methods are disclosed for diagnosing and treating breast cancer.

### SEQUENCE LISTING

The instant application contains a Sequence Listing which has been submitted electronically in ASCII format and is hereby incorporated by reference in its entirety for all purposes. The ASCII copy, created on Nov. 29, 2023, is named 106549-780334 CU5633H-US1_SL and is 6,731 bytes in size.

### BACKGROUND

Cancer continues to be a leading cause of death worldwide. Breast cancer is the most common cancer in women with about 1.68 million new cases and 520,000 deaths worldwide annually. Of these, estrogen receptor positive breast cancers (ER+BC) are the highest percentage of breast cancers at about 70 percent with a relapse within ten years in about 20 percent of the cases. While some specific therapies have been developed for certain cancers, the distinct physiology, and unique characteristics of certain cancers as well as the growing need for cancer treatments have created a need for more specifically targeted treatments for cancer.

### SUMMARY

Some embodiments of the present disclosure are based on development of anti-Semaphorin 7A (referred to herein as SEMA7A; also referred to as CD108, or Sema KI or Sema L) antibodies and their uses in the diagnosis and treatment of health conditions. In certain embodiments, the health conditions are linked to expression of or over-expression of, SEMA7A. In accordance with these embodiments, these antibodies have a high binding affinity and specificity to SEMA7A and can inhibit SEMA7A expression and/or activity and reduce SEMA7A effects on cell proliferation, metastasis, and/or pro-inflammatory-related side effects, as well as other SEMA7A pro-tumor activities.

In some embodiments, an isolated SEMA7A antibody provided herein can include polyclonal or monoclonal antibodies. In other embodiments, an antibody against SEMA7A activities can include a monoclonal antibody raised against one or more SEMA7A polypeptide fragment where the antibody is capable binding to SEMA7A and reducing or eliminating SEMA7A activities (e.g., pro tumor or pro-metastasis activity). In some embodiments, a SEMA7A polypeptide fragment is about 75% or more, or up to 100% identity to SEQ ID NO:2 or include at least about 75% up to 100% in identity to amino acids 381-392 of the polypeptide represented by SEQ ID NO: 1 or isomer thereof or other SEMA7A molecule thereof. In certain embodiments, the polypeptide fragment construct for use in generating antibodies disclosed herein does not include consecutive amino acids 371-380 or fragment thereof, consecutive amino acids 393-411 or fragment thereof, or both of the polypeptide represented by SEQ ID NO:1. It is contemplated that truncated or mutated versions of these polypeptides can form part of a construct including 75% or more up to 100% identity to SEQ ID NO:2 or include at least 75% identity to 100% identity to amino acids 381-392 of the polypeptide represented by SEQ ID NO: 1 or isomer thereof. In certain embodiments, the peptide or epitope of SEMA7A of use to generate an antibody against SEMA7A is 100% identical to SEQ ID NO:2 or SEQ ID NO:3 or raised against a conjugate thereof. In other embodiments, the antibody is a monoclonal antibody able to bind this epitope. In yet other embodiments, these monoclonal antibodies can be used in pharmaceutical compositions administered to inhibit SEMA7A pro-tumor activities.

In other embodiments, monoclonal antibodies can be raised against the one or more SEMA7A polypeptide fragments contemplated herein. In some embodiments, a SEMA7A polypeptide fragment is about 75% or more, or up to 100% identity to SEQ ID NO:2 or include at least about 75% up to 100% in identity to amino acids 381-392 of the polypeptide represented by SEQ ID NO: 1 or isomer thereof or other SEMA7A molecule thereof. In certain embodiments, the polypeptide fragment construct for use in generating antibodies disclosed herein does not include consecutive amino acids 371-380 or fragment thereof, consecutive amino acids 392-411 or fragment thereof, or both of the polypeptides represented by SEQ ID NO: 1. It is contemplated that truncated or mutated versions of these polypeptides can form part of a construct including 75% or more up to 100% identity to SEQ ID NO:2 or include at least 75% identity to 100% identity to amino acids 381-392 of the polypeptide represented by SEQ ID NO: 1 or isomer thereof. In certain embodiments, the peptide or epitope of SEMATA of use to generate a monoclonal antibody against SEMA7A is 100% identical to SEQ ID NO:2.

In other embodiments, one or more peptides disclosed herein can be of use alone as a peptide or form part of a conjugate or form part of a fusion polypeptide (e.g., having a protein transduction domain such as tat or an Fc or other suitable domain or conjugate) to generate one or more antibody disclosed herein. In accordance with these embodiments, these peptides or conjugates or fusion polypeptides further include a linker or molecule to separate the peptide of interest from the conjugate or secondary molecule for improved antibody production. Adjuvants or other immune stimulators known in the art can be used in combination with the peptide or conjugated peptide in order to enhance antibody production in the host animal. In other embodiments, antibodies generated from these peptides can form part of a pharmaceutical composition and can be introduced to a subject to diagnose or treat a health condition at least partially caused by SEMA7A expression or over expression. In certain embodiments, one or more peptides having at least 75% homology or up to 100% identity to the amino acid sequence represented by SEQ ID NO: 2 or SEQ. ID NO:3 are contemplated for generating antibodies disclosed herein. In certain embodiments, these antibodies form part of these pharmaceutical compositions of use for diagnosing and/or treating a subject having cancer or other health condition having adverse SEMA7A involvement. Any method for introducing or administering an antibody-containing composition to a subject in need thereof is contemplated.

In certain embodiments, antibodies (e.g., monoclonal antibodies) disclosed herein directed to bind SEMA7A can be used to treat health conditions associated with this molecule. In other embodiments, antibodies (e.g., monoclonal antibodies) disclosed herein directed to bind SEMA7A can be used to diagnose a health conditions associated with this molecule. In some embodiments, concentrations of SEMA7A in a sample from a subject can be assessed. In other embodiments, changes in concentrations of SEMA7A in samples from a subject can be assessed in order to monitor treatment regimens for further adjustment to a treatment regimen.

In some embodiments, the health condition to be treated disclosed herein includes, but is not limited to, health conditions concerning cancer. In other embodiments, the health condition to be treated disclosed herein includes, but is not limited to, neurological development, neurological conditions, wound healing, bone homeostasis conditions, angiogenesis issues, pro-inflammatory cytokine release and regulation-related conditions, dendritic cell-related adverse effects on adhesion and motility, fibrosis-related conditions, effects on tumor growth, progression and metastasis where inhibition of SEMA7A expression and/or activities reduces the effects of, prevents, or treats the condition. In some embodiments, cancers can be treated with SEMA7A antibodies having at least 75% identity up to 100% identity to SEQ ID NO:2 disclosed herein so. In some embodiments, antibodies to SEMA7A disclosed herein can reduce tumor progression, reduce or inhibit metastasis, shrink tumors, induce tumor cell death, reduce tumor expansion, reduce lymphangiogenesis/angiogenesis lymphovascular invasion and/or reduce lymphogenous/hematogenous metastasis (e.g. reduce infiltration or metastasis of tumor cells into lymph nodes or distant organs) In other embodiments, antibodies against SEMA7A disclosed herein can be used to reduce recruitment of suppressive immune cells such as macrophages and to reduce fibrillar collagen and fibronectin deposition in the tumor microenvironment. In yet other embodiments, SEMA7A presence or levels can be detected in a tumor to assess tumor levels or migration. In accordance with these embodiments, if SEMA7A is present or the level has reached a predetermined threshold, the tumor can be treated with one or more SEMA7A I polyclonal or monoclonal antibodies to SEQ ID NO, 2 or fragment thereof, to reduce tumor expansion, shrink tumor size and/or reduce tumor metastasis.

In some embodiments, inhibitors of SEMA7A expression or activities (e.g., tumor promoting activities) can include any SEMA7A inhibitor or specified antibody or monoclonal antibody thereof. In other embodiments, inhibitors of SEMA7A expression or activities can include, but are not limited to, antibodies including monoclonal, polyclonal or antibody fragment thereof, anti-sense RNA, siRNA, or use of any editing technology such as Crispr or other gene editing technology capable of genetically manipulating SEMA7A to edit SEMA7A by completely removing it or editing it to reduce its pro-tumor activities, for example.

In other embodiments, an isolated SEMA7A antibody or monoclonal antibody disclosed herein binds to an epitope on a polypeptide having an amino acid sequence represented by SEQ ID NO: 2 or 3 or isomer equivalent thereof or fragment thereof where binding the epitope inhibits SEMA7A expression and activities. In some embodiments, the isolated SEMA7A antibody or monoclonal antibody disclosed herein binds to an epitope on a polypeptide having an amino acid sequence represented by SEQ ID NO: 2 or 3 or isomer equivalent thereof or fragment thereof and are capable of killing tumor cells or inducing apoptosis of tumor cells. In certain embodiments, the tumor cells are breast cancer cells. In some embodiments, the isolated SEMA7A antibody or monoclonal antibody is a full-length antibody or SEMA7A binding fragment thereof. In some embodiments, the isolated anti-SEMA7A antibody is a monoclonal antibody. In certain embodiments, the isolated anti-SEMA7A antibody is a single-chain antibody (scFv).

In other embodiments, a polynucleotide encoding the SEMA7A antibody, monoclonal antibody or antigen binding fragment thereof provided herein is contemplated. In accordance with these embodiments, the polynucleotide can further include a vector for expressing the encoded antibody or antigen binding fragment. In yet other embodiments, host cells having the polynucleotide encoding an SEMA7A antibody, monoclonal antibody or antigen binding fragment thereof are included herein.

In some embodiments, pharmaceutical compositions are disclosed containing at least one anti-SEMA7A antibody, monoclonal antibody or polynucleotide expressing the at least one antibody as described herein (e.g., that binds to a sequence that is at least 75% up to 100% homologous to the sequence represented by SEQ ID NO:2, or fragment thereof) and a pharmaceutically acceptable carrier of use for treating health conditions are provided. In accordance with these embodiments, the pharmaceutical compositions can further include an anti-inflammatory agent, an anti-cancer agent, a non-specific innate immune response stimulator, or other standard agent of use in combination with the antibodies and monoclonal antibodies disclosed herein, for example to treat conditions having expression or over-expression of SEMA7A. It is noted that the antibodies, monoclonal antibodies, and fragments thereof bind to any SEMA7A including any isomeric form of SEMA7A. In some embodiments, the pharmaceutical compositions can further include one or more of an anti-microbial agent, a chemotherapeutic agent, and/or immunostimulatory or anti-inflammatory agent depending on the health condition to be treated. Combination therapies that include antibodies disclosed herein are contemplated as well as combining antibody treatments with standard surgical procedures for treating the health condition. In yet other embodiments, the composition or pharmaceutical compositions disclosed herein can include a monoclonal antibody clone designated as H1, H7 or fragment thereof, or a combination thereof of use in single or multiple dosing regimens to treat a subject in need thereof of such a treatment.

In other embodiments, methods for using pharmaceutical compositions including, but not limited to, one or more SEMA7A antibody, monoclonal antibody or fragment thereof; and a pharmaceutically acceptable carrier for treating, reducing and/or preventing a condition associated with SEMA7A expression or overexpression is contemplated. In some embodiments, methods include administering a composition disclosed herein and inducing anti-tumor activities in the subject in need thereof. In accordance with these embodiments, pharmaceutical compositions described herein can be administered to the subject by any means known in the art for administering an antibody, monoclonal antibody or fragment thereof. In another embodiment, methods are provided for diagnosing and/or treating cancer in the subject. In accordance with these embodiments, methods for treating cancer in the subject include, but are not limited to, administering to a subject in need thereof an effective amount of the pharmaceutical composition described herein. In other embodiments, the cancer includes a solid tumor. In other embodiments, the solid tumor includes, but is not limited to, breast, prostate, liver, lung, kidney, stomach, ovarian, head, neck, brain, skin, testicular, pancreatic or other solid tumor. In certain embodiments, when treating solid tumors, the pharmaceutical compositions can be administered systemically, topically, by bolus infusion, or by direct local administration into the solid tumor of the subject.

In some embodiments, compositions and methods for inhibiting SEMA7A expression and/or activity disclosed herein can be used to reduce or prevent expansion, migration, metastasis and/or maturation of cancer stem cells. In certain embodiments, the cancer stem cells include any cancer stem cell of any origin. In other embodiments, the cancer stem cells include any cancer stem cell resistant to radiation, chemotherapy or other anti-cancer treatment where compositions and methods disclosed herein can be used to reduce or prevent cancer stem cell activities and survival to fully treat a subject in need thereof. In other embodiments, cancer stem cells disclosed herein can include mammary cells and/or cells of mammary tissues (e.g., epithelial cells). In another embodiment, compositions and methods disclosed herein can be used to inhibit metastasis of cancer stem cells, treat a tumor and/or reduce anoikis resistance in a subject. In certain embodiments, the subject is a female subject having post-partum breast cancer (PPBC). In certain embodiments, antibodies against SEMA7A can be used to reduce or prevent expansion, migration, metastasis and/or maturation of cancer stem cells. In some embodiments, compositions and methods disclosed herein can be used to reduce expression and/or activity of SEMA7A in normal cells in order to treat a subject.

In other embodiments, kits are contemplated for transport, storage and use of polyclonal or monoclonal antibodies or combination therapies disclosed herein. In some embodiments, kits can include additional agents for diagnosing and/or treating a health condition in need of reducing SEMA7A expression and/or activity.

### BRIEF DESCRIPTION OF THE DRAWINGS

The following drawings form part of the present specification and are included to further demonstrate certain embodiments of the present disclosure. Certain embodiments can be better understood by reference to one or more of these drawings in combination with the detailed description of specific embodiments presented herein.

FIGS. 1A-1B represent (A) a schematic diagram illustrating a protein structure of SEMA7A and (B) a representative SEMA7A sequence with the epitope of interest bolded and underlined and a schematic of the protein domains according to various aspects of the disclosure.

FIGS. 2A-2P represent exemplary plots demonstrating some outcomes from measuring SEMA7A expression in human breast tumor tissues according to various aspects of the disclosure. These figures represent quantitative immunohistochemistry for medium+strong (M+S) staining for SEMA7A in normal (A) adjacent breast tissues; PPBCs separated by years post childbirth (B) and quantification of tumors from the same patients (C and D); And further that SEMA7A expression does not differ with breast cancer clinical characteristics. % SEMA7A medium+strong (M+S) staining in cohort of patients with nulliparous cases denoted as open squares and postpartum cases as closed grey squares. (E) Stage, (F) Lymph node involvement, (G) Lymphovascular invasion, (H) Grade, (I) ER status, and (J) Tumor biological subtype. (K) Cases with gravidity >0 were removed in the nulliparous group. L represents SEMA7A expression separated by number of children; SEMA7A expression in Nulliparous cases (open squares) and postpartum breast cancers (grey closed squares) separated by (M) Stage, (N) Grade, and (O) Tumor Biological subtype. A-nova test; SEMA7A expression and analysis of absence or presence of any recurrence (local, regional, and metastatic) in nulliparous (open squares) and postpartum (grey closed squares) groups according to various aspects of the disclosure.

FIGS. 3A-3D represent exemplary plots (A-C) of SEMA7A levels in different patient populations and a survival curve (D) illustrating some outcomes from measuring SEMA7A and responses in human patients having breast tumors expressing SEMA7A according to various aspects of the disclosure.

FIG. 4A-4B (A) represents an exemplary plot demonstrating some results from antibody measurements of SEMA7A in human blood samples; (A) SEMA7A expression (˜36% positive by IHC) predicts for breast cancers in nulliparous (nullip) patients or patients with postpartum breast cancer (PPBC) and (B) comparison of SEMA7A expression in tumor by immunohistochemistry (IHC) compared to SEMA7A concentrations in patient serum according to various aspects of the disclosure.

FIGS. 5A-5E represent an exemplary plot demonstrating some results from measuring cell viability (A) and invasion (representing metastasis, (B) following exposure of cancer cells with an SEMA7A antibody or a saporin-conjugated SEMA7A antibody and (C and D) percent confluent cells in the presence of increasing anti-SEMA7A antibody (mid versus high expressing); and (E) number of cells invaded in the presence or absence of various SEMA7A antibodies and controls according to various aspects of the disclosure.

FIG. 6 represents an exemplary schematic of membrane-bound SEMA7A and shed SEMA7A in a tumor cell according to various aspects of the disclosure.

FIGS. 7A-7C represent an exemplary (A) Western blot depicting recognition of all three isoforms of SEMA7A by mouse monoclonal antibody generated using an immunizing peptide (e.g., 10-mer) and termed SmAbH1 (right panel). Balb/c mice were inoculated with mouse mammary tumors followed by monoclonal antibody treatments for SEMA7A (SmAbH1); (B) mice were inoculated with mouse mammary tumor cells and later treated with 2 different SEMA7A monoclonal antibodies raised against the 10-mer. (C) mice were inoculated with mouse mammary tumor cells and later treated with a SEMA7A monoclonal antibody disclosed herein and studied for effect according to various aspects of the disclosure.

FIGS. 8A-8B illustrates representative survival curves; (A) survival curves for mice treated with monoclonal antibodies against SEMA7A (H1 and H7) from FIG. 7B; and (B) survival analysis for time to tumor size of >200 mm³ in mice treated with SmAbH1, or IgG control, from FIG. 7C according to various aspects of the disclosure.

FIGS. 9A-9D are graphical representations of mice from FIG. 7C with respect to immune cell activation. (A) represents analysis of percent immune cells within a representative tumor using flow cytometry for commonly observed immune cell-associate leukocyte antigen (CD45); (B) represents an analysis of percent CD4+ T cells of CD45+ immune (CD45+B220-CD3+CD8−) cells observed using flow cytometry; (C) represents analysis of percent CD8+ T cells of CD45+ immune (CD45+B220−CD3+CD4−) cells using flow cytometry; and (D) represents analysis of activated T cells, based on expression of interferon gamma (IFNγ) and tumor necrosis factor alpha (TNFα) on CD8+ T cells by flow cytometry. * p<0.05, ** p<0.01, t-test according to various aspects of the disclosure.

FIGS. 10A-10E represents graphical illustrations of exemplary data for (A) survival statistics for time to tumor size to >500 mm³ in mice injected with tumors and treated with a commercially available anti-tumor antibody (e.g., anti-PD-L1, anti-PD-1), SEMA7A monoclonal antibody monotherapy or a combination therapy; (B) represents exemplary tumor growth curves when tumor size is about 150 mm³ treated with a commercially available anti-tumor antibody (e.g., anti-PD-L1, anti-PD-1), SEMA7A monoclonal antibody monotherapy or a combination therapy (black arrows indicate treatments); (C) illustrates individual tumor volumes from each group pre-treatment and at study end; (D) illustrates percent of mice with evidence of lung or lymph node metastasis in each treatment group; and (E) mouse mammary tumor growth in mice treated with a commercially available anti-tumor antibody monotherapy starting after tumor establishment in the mice where percent of mice with metastasis at the study end is represented to the right of each experimental plot according to various aspects of the disclosure.

FIGS. 11A-11H represents various graphs and images of tumor analysis and treatment according to various aspects of the disclosure. (A) represents analysis for relapse free survival of breast cancer patients treated with endocrine therapy assessed for SEMA7A levels; (B) MCF7 tumor cell growth in mice treated with endocrine therapy (e.g., Fulvestrant); (C) Lung metastasis of MCF7 in mice from 11B; (D) is a representative image illustrating BCL2 expression in SEMA7A OE cells; (E) MCF7 tumor growth in mice treated with endocrine therapy and a Bcl-2 inhibitor (e.g., Venetoclax) indicated by the arrows; (F) Lung metastasis in mice from D above; (G) illustrates clonogenic growth of MCF7 EV or SEMA7A OE cells treated with 5 μM SU6656; clonogenic data are normalized to vehicle controls; and (H) represents clonogenic growth of MCF7 EV or SEMA7A OE cells treated with a composition according to various aspects of the disclosure.

FIGS. 12A-12F demonstrates support that SEMA7A promotes anoikis resistance in normal non-cancerous cells (e.g., MECs) where (A) represents cleaved caspase 3/7 (CC3/7) expression in example cell populations, MCF10A EV or S7a-OE cells after 24 hours in attached or forced suspension cultures; (B) illustrates SEMA7A expression by flow cytometry in attached and forced suspension control cell cultures, MCF12A; (C) CC3/7 in MCF12A cells in forced suspension+/−exogenous S7a protein and/or ITGB1 blocking antibody, 9eg7; (D) represents flow cytometry analysis of pAKT in insulin or S7a-treated MCF12A with representative flow plots; (E) represents flow cytometry analysis regarding percent SEMA7A+ mammary epithelial cells from tissues harvested from nulliparous (N) or recently weaned C57/BL6 mice at involution days 1-28 (11-128); and (F) illustrates flow cytometry of live epithelial cells as measured by Aquazombie-EpCAM+ in mammary tissues harvested from recently weaned WT or Sema7a^{tm1Alk/J} mice according to various aspects of the disclosure.

FIGS. 13A-13D illustrates that inhibition or loss of SEMA7A promotes alveolar collapse through changes in cell survival and adipocyte repopulation. Immunohistochemical quantification of mammary glands from wild type (WT) or SEMA7A^{tmlAlk/J} mice at 1, 2, 3 and 6 days post forced involution. Quantification of (A) alveolar area (B) pSTAT3 (C) cleaved caspase-3; and (D) perilipin staining are represented according to various aspects of the disclosure.

FIGS. 14A-14G illustrates various graphs and plots of analysis SEMA7A induced survival of cancer cells and effects thereof. As discovered herein SEMA7A promotes survival via integrins and enriches for pAKT+ cells. (A-E) illustrates flow cytometry analysis of mammary glands from WT (left) and SEMA7A^{tmlAlk/J} (right) mice; (A) Stratification of MEC subtypes by EpCAM/CD49f staining; (B) percent EpCAMhiCD49f+ LPCs of EpCAM+ cells; (C) percent SEMA7A+ of live (Aquazombie−) EpCAMhiCD49f+ LPCs; (D) percent ITGB1+ITGA6+ of EpCAM cells; (E) percent SEMA7A of ITGB1+ITGA6+ cells; (F) CC3/7 expression in MCF12A cells+/−S7a and/or GoH3; (G) percent pAKT in MCF12A cells+/−S7a and/or 9eg7/GoH3, according to various aspects of the disclosure.

FIGS. 15A-15G represents graphs and images of analysis of mechanisms related to SEMA7A and ITGB1/ITGA6 promoting transformation and chemoresistance of tumor cells according to various aspects of the disclosure. (A) illustrates percent celltrace violet bright of singlets in MCF10A EV and OE cells; (B-C) illustrate number of mammospheres formed by MCF12A cells+/−S7a (SEMA7A) and GoH3 or 9eg7; (D) illustrates number of colonies formed in soft agar assay; (E, F G) illustrate 10ADCIS KD or OE mammospheres cultured in paclitaxel+/−9eg7 or GoH3 where (E) represents a graph of number of mammospheres'; (F) illustrates percent CD44+CD24− of singlets by flow cytometry; and (G) are representative stained images, according to various aspects of the disclosure.

FIGS. 16A-16B illustrate in (A) that monoclonal antibodies to SEMA7A generated from a peptide disclosed herein reduces mammosphere formation; and (B) cell death in vitro (blue=control above the solid grey plot and red=SEMA7A OE above the clear plots) according to various aspects of the disclosure.

FIGS. 17A-17E illustrate that SEMA7A is upregulated in multiple breast cancers and is a predictor for decreased relapse-free survival when expressed with another cancer marker (e.g., α6-integrin). (A-D) illustrate TCGA data for Sema7a in (A) normal vs primary tumor; (B) normal versus stages I-IV; (C) normal versus cancer stratified by age; (D) normal versus breast cancer subclasses (e.g. luminal: HER2+, TNBC-BL1, TNBC-BL2, TNBC-IM, TNBC-LAR, TNBC-MSL, TNBC-M, TNBC-UNS); and (E) Kaplan-Meier 5-year relapse-free survival analysis for Sema7a in Itga6 low (n=2032) or high (n=2032) expressing patients according to various aspects of the disclosure.

FIGS. 18A-18B illustrates effects of control versus SEMA7A monoclonal antibodies versus control on tumor volume (A) and invasion scores by staining analysis on harvested tissues from A according to various aspects of the disclosure.

FIGS. 19A-19C illustrates cell viability in ER+(A) human derived Michigan Cancer Foundation 7 (MCF7); (B) mouse derived TC11 and (C) mouse derived SSM2 cells (selected for responsiveness to estrogen and to inhibitors of estrogen receptor or agents able to block the estrogen receptor) were treated for 48 hours with either IgG control, an estrogen receptor antagonist or degrader (e.g., fulvestrant (Fulv)), SEMA7A monoclonal antibody (e.g., SmAb H1) or the combination thereof according to various aspects of the disclosure.

FIG. 20 illustrates a studied parameter of pro-tumor cells, percent confluence in cell culture of tumor cells (e.g., 66cl4). This figure illustrates a time course study of confluence over time in the presence of control or increasing concentrations of SEMA7A monoclonal antibody (mAb, SmAbH1) demonstrating dose dependency where reduced levels of SEMA7A affected confluence according to various aspects of the disclosure.

FIG. 21A-21C illustrates percent confluence of control (mouse derived 66cl4 DDK) and SEMA7A OE (DDK SEMA7A) cells treated with (A) SEMA7A monoclonal antibody (mAb, SmAbH1), (B) commercially available SEMA7A clone (Santa Cruz, D4, #376149); (Santa Cruz, D4, Catalog #sc-376149; immunizing peptide represented by SEQ ID NO:4: QPIPTETFQVADRHPEVAQRVEPMGPLKTPLFHSKYHYQKV); and (C) another SEMA7A polyclonal antibody (Abcam; Ab23578; immunizing peptide within residues 1-100) according to various aspects of the disclosure. Based on previous observations it is likely that the decreased confluence is due to antibody blocking the SEMA7A promoting effects on cell growth, cell survival and stem cell phenotypes

### DEFINITIONS

As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “treat,” “treating,” “treatment” and the like, unless otherwise indicated, can refer to reversing, alleviating, inhibiting the process of or expansion of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the condition, or disorder.

An “epitope” as used herein can refer to the site on a target antigen that is recognized and bound by an antibody. In some embodiments, the site can be entirely composed of amino acid components, entirely composed of chemical modifications of amino acids of the protein (e.g., glycosyl moieties), or composed of combinations thereof. In some embodiments, overlapping epitopes can include at least one common amino acid residue. In some embodiments, two or more SEMA7A antibodies or monoclonal antibodies or epitope-binding fragment thereof described herein can bind to the same epitope or a substantially overlapping epitope (e.g., containing less than 3 non-overlapping amino acid residues, less than 2 non-overlapping amino acid residues, or only 1 non-overlapping amino acid residue). In certain embodiments, the epitope is represented by SEQ ID NO:2 or SEQ ID NO:3 or biologically active fragment thereof of at least 4 consecutive amino acids or at least 5, or at least 6, or at least 7, or at least 8, or at least 9 consecutive amino acids. In certain embodiments, an immunizing peptide or epitope of use to generate antibodies can be a repeated sequence of at least 2 repeated polypeptide epitopes, at least 3 polypeptides, at least 4 polypeptides or more linked together by any means (e.g., a linker). These polypeptides can be linked at the amino or carboxy terminus or a mixture or alternating consecutive polypeptides together to improve antibody production and/or specificity.

Certain constructs and polypeptides are described below in terms of “percent identity” or “percent sequence identity” or “percent homology” or “percent sequence homology” to a reference sequence. When used herein, the term “percent identity” or “percent sequence identity” or “percent homology” of two amino acid sequences can be determined by any method known in the art.

Constructs and polypeptides having a certain percent sequence homology to a reference sequence can have one or more “conservative amino acid substitutions” relative to the reference sequence. These “conservative amino acid substitution(s)” can refer to one or more amino acid substitution that does not alter the relative charge or size characteristics of the protein in which the amino acid substitution is made. In some embodiments, variants herein can be prepared according to methods for altering polypeptide sequence known to one of ordinary skill in the art.

### DETAILED DESCRIPTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times, and other specific details can be modified through routine experimentation. In some cases, well known methods, or components have not been included in the description.

Embodiments of the instant disclosure relate to compositions, methods of making and methods of using antibodies including monoclonal antibodies against a pre-selected peptide or active fragments thereof, of SEMA7A. In some embodiments, the antibodies disclosed herein are capable of binding to SEMA7A and treating a condition expressing or over-expressing SEMA7A. In accordance with these embodiments, SEMA7A monoclonal antibodies can be generated against SEQ ID NO:1. In other embodiments, one or more constructs including SEQ ID NO: 1 or constructs at least 75% homologous thereof, can be used to generate one or more antibodies (or monoclonal antibodies) against the one or more constructs. In accordance with these embodiments, the one or more antibodies can be used to target SEMA7A in a health condition to treat a subject having such a health condition.

Other embodiments relate to compositions, methods of making and methods of using antibodies for treatment and/or diagnosis including monoclonal antibodies against a pre-selected peptide or active fragments thereof, represented by SEQ ID NO: 2 or SEQ ID NO:3, or biologically active fragment thereof of SEMA7A. In some embodiments, these monoclonal antibodies disclosed herein are capable of binding to SEMA7A and treating a condition expressing or over-expressing SEMA7A. In certain embodiments, a monoclonal against a polypeptide conjugated to a carrier (e.g., BSA or KLH) or other molecule or fusion polypeptide of the polypeptide represented by SEQ ID NO: 2 or SEQ ID NO:3 or simply the polypeptide represented by SEQ ID NO: 2 or SEQ ID NO:3 is used to generate and use monoclonal antibodies to diagnose and/or treat a subject having a health condition associated with expression of, or over-expression of SEMA7A. In accordance with these embodiments, SEMA7A monoclonal antibodies can be generated against SEQ ID NO:2 or 3. In other embodiments, one or more constructs including SEQ ID NO:2 or 3 or constructs of at least 75% homologous thereof, can be used to generate one or more antibody (or monoclonal antibody) against the one or more constructs. In accordance with these embodiments, the one or more antibodies can be used to target SEMA7A to treat a health condition in a subject having such a health condition. In certain embodiments, the monoclonal antibodies generated to recognize and bind to SEQ ID NO:2, or 3, or biologically active fragment thereof can be used in a therapeutic composition to diagnose and/or treat cancer (e.g., leukemia, breast cancer; PPBC, pancreatic cancer) to reduce or eliminate the occurrence of relapse and/or treat the cancer in the subject. In other embodiments, these monoclonal antibodies can be used to target and kill tumor cells including, non-stem cell cancer cells and cancer stem cells in a subject in need thereof. In yet other embodiments, monoclonal antibodies, or biologically active fragment thereof against SEQ ID NO:2 or 3 can be used in combination with one or more cell survival pathway inhibitor (e.g., BCL-2 inhibitor, P13K pathway inhibitor, or the like) and/or one or more of a beta-1 integrin or Akt inhibitors. In some embodiments, combination therapies can include monoclonal antibodies, or biologically active fragment thereof against SEQ ID NO:2 or 3 and one or more PI3K (e.g., LY294002 or similar) inhibitor and/or Src inhibitor (e.g., SU6656) in order to reduce tumor cell viability or eliminate tumor cells and/or cancer stem cells expressing SEMA7A or other marker. In certain embodiments, these treatments can be timed to treat post-partum breast cancer patients at risk, or breast cancer patients at high risk of breast cancer relapse. In accordance with these embodiments, combinations disclosed herein can target and kill tumor cells by blocking the mechanisms of action by which SEMA7A promotes tumor cell growth and survival.

In certain embodiments, SEMA7A monoclonal antibodies generated to bind to the amino acid sequence represented by SEQ ID NO:2 or SEQ ID NO:3 or biologically active fragment thereof can be used to treat a health condition having SEMA7A expression or over-expression or SEMA7A activity such as tumor promotion and expansion. In some embodiments, monoclonal antibody clones contemplated herein can include clones referenced as H1 or H7 or other monoclonal antibody thereof. Other monoclonal antibody clones are contemplated of use in compositions and methods disclosed herein generated to the peptides of some embodiments provided herein. In certain embodiments, SEMA7A monoclonal antibody clones generated against SEQ ID NO:2 or SEQ ID NO:3 or biologically active fragment thereof and/or referenced herein as monoclonal antibody clone H1 and/or monoclonal antibody referenced herein as clone H7 can be used to treat cancer. In other embodiments, monoclonal antibodies or polyclonal antibodies generated against SEQ ID NO:2 or SEQ ID NO:3 or biologically active fragment thereof or clone H1 and/or clone H7 can be used to diagnose cancer (e.g., breast cancer, leukemia, pancreatic cancer, or other cancer overexpressing or expressing SEMA7A). In certain embodiments, polyclonal or monoclonal antibodies generated against SEQ ID NO:2 or SEQ ID NO:3 or biologically active fragment thereof can further be used to reduce tumor progression, tumor metastasis, tumor expansion and/or tumor volume. In certain embodiments, the cancer is any cancer expressing SEMA7A or other condition expressing SEMA7A. In other embodiments, the cancer is a solid tumor expressing SEMA7A. In other embodiments, the cancer is a non-solid cancer of the blood (e.g., leukemia). In yet other embodiments, the cancer is breast cancer. In certain embodiments, the breast cancer is ER+BC, ER-BC, or postpartum breast cancer (PPBC). In certain embodiments, the PPBC breast cancer is diagnosed with 1, to within 2, to within 3, to within 5, to within 10 years of childbirth. In certain embodiments, monoclonal antibodies generated against SEQ ID NO:2 are used to diagnose cancer and diagnose cancer progression, relapse and/or cancer survival. In some embodiments, the cancer is breast cancer. In other embodiments, monoclonal antibodies generated against SEQ ID NO:2 are used to treat cancer, treat cancer progression, reduce, or eliminate cancer relapse and/or improve cancer survival. In some embodiments, the cancer is breast cancer. In some embodiments, the monoclonal antibodies are part of a pharmaceutical composition. In accordance with these embodiments, the pharmaceutical composition can be used to treat breast cancer and reduce tumor progression, tumor metastasis, tumor expansion and/or tumor volume. In other embodiments, the breast cancer is ER+BC, ER-BC, or postpartum breast cancer (PPBC), In some embodiments, the pharmaceutical composition disclosed herein can be used to treat breast cancer and reduce or prevent lymphovascular invasion (LVI) and lymph node(s) (LN) involvement. In certain embodiments, the breast cancer is ER+BC, ER-BC, or postpartum breast cancer (PPBC). In certain embodiments, the PPBC breast cancer is diagnosed within 1, to within 2, to within 3, to within 5, to within 10 years of childbirth.

In certain embodiments, monoclonal antibodies generated against SEQ ID NO:2 or SEQ ID NO:3 can be part of a pharmaceutical composition and used to treat a subject having breast cancer to kill breast cancer cells including non-stem and stem cell cancer cells. In accordance with these embodiments, the subject can be treated daily, weekly, monthly, or other regimen or at a time when SEMA7A levels are detectable, are used to diagnose cancer and diagnose cancer progression, relapse and/or cancer survival. In some embodiments, the cancer is breast cancer.

### SEMA7A

SEMATA is a member of the Semaphorin family of axon guidance molecules and, when expressed on human erythrocytes, is the John Milton Hagen (JMH) blood group antigen. SEMA7A is an ˜80 kDa membrane-anchored glycoprotein that contains an RGD integrin interaction motif within its full-length amino acid sequence.

### Cancers

Cancer is one of the leading health issues globally and one of the leading causes of death in humans and other mammals. Breast cancer, for example, is a global health threat with an estimated 1.7 million cases annually. Death due to breast cancer disproportionately affects low income countries and younger women in these countries are particularly at risk. In the U.S., about 27,000 younger women are affected by breast cancer annually, with age of less than 35 years old and diagnosis within ten years of most recent childbirth as two unique risk factors for breast cancer metastasis and death. Pregnancy-associated breast cancer (PABC) has been defined as any breast cancer diagnosed during pregnancy or in the 1-2 years immediately after childbirth. However, significant research has clarified that women diagnosed with breast cancer during pregnancy fare equal to their non-pregnant peers with similar breast cancers. However, women diagnosed in the early postpartum years face significant increased risk for metastasis in comparison to nulliparous women or women whose children are older, despite their breast cancers having similar risk profiles. Therefore, in certain instances it is important to separate PABC, especially those diagnosed during pregnancy, from postpartum breast cancer (PPBC) as the outcomes can vary in these groups.

PPBC is currently defined as a breast cancer diagnosis occurring within ten years following childbirth. In one study, patients diagnosed with PPBC experienced a 2-fold increase in mortality risk when diagnosed 4-6 years following childbirth, while patients within two years postpartum experienced a thirty percent increase in mortality rate. In other studies, where detailed tumor characteristics were available, patients diagnosed within 5 years of giving birth had about a three-fold risk of metastatic recurrence without a significant difference present in the clinical risk characteristics of the tumors, as compared to nulliparous or patients whose most recent childbirth occurred more than ten years before diagnosis. This increased risk period was then extended when a larger cohort study demonstrated that postpartum patients diagnosed with Stage 1 and 2 tumors up to ten years from their most recent childbirth were at a 3 to 5-fold significantly higher risk for developing metastases, with tumors continuing to recur up to 15 years after diagnosis. In these studies, a postpartum diagnosis was associated with a higher incidence of having recurrence and poor treatment response as well as metastasis. In certain embodiments, treatments disclosed herein can reduce or eliminate metastases, reduce, or eliminate lymphovascular invasion (LVI) and/or reduce or eliminate lymph nodes (LN) lymphovascular invasion (LVI) and involved lymph node(s) (LN), involvement, or a combination thereof in cancers expressing or over-expressing SEMA7A.

In certain embodiments, antibodies disclosed herein can be used to enhance anti-tumor responses to shrink or eliminate cancerous growth and/or reduce or prevent tumor expansion. In certain embodiments, the anti-SEMA7A antibodies disclosed herein can be used in conjunction with another appropriate active agent (e.g., a chemotherapeutic agent or other targeted anti-cancer therapy, an antibiotic, an anti-fungal, an anti-viral, or a non-specific immune stimulator). In some embodiments, compositions disclosed herein can be used to diagnose the presence of SEMA7A in a subject having cancer which correlates with unresponsiveness to standard therapies and relapse in certain cancer patients which can reduce or eliminate needless treatment with more toxic agents or treatment such as radiation or chemotherapy depending on the tumor type. In certain embodiments, detection of SEMA7A in a subject having breast cancer and/or PPBC can alert the caretaker and eliminate treatment regimens known to not be unsuccessful in treating these tumors, avoiding anti-cancer toxicities in order to focus on treatments like cell survival pathway inhibitors and/or blocking SEMA7A using an antibody treatment disclosed herein to treat the subject.

Alternatively, antibody or monoclonal antibody therapies disclosed herein can be used before, after or simultaneously with other treatments such as radiation, chemotherapy, anti-inflammatory or other targeted anti-cancer therapies or immunotherapies. In some embodiments, antibodies to SEMA7A disclosed herein can be administered in combination with a non-specific immune stimulator composition, containing, for example, a TLR ligand, T-cell stimulant, or other immunostimulatory such as CTLA-4 or the like or anti-cancer agent during, at the time of, or after treating a tumor with one or more antibodies disclosed herein. In accordance with these embodiments, these combinations can act synergistically to enhance anti-tumor or anti-SEMA7A activities in order to treat cancer or other health condition. In certain embodiments, antibodies generated against SEQ ID NO:2 and/or SEQ ID NO:3 of SEMA7A can be used in combination therapies disclosed herein. In certain embodiments, antibodies generated against SEQ ID NO:2 and/or SEQ ID NO:3 of SEMA7A can be used in multiple doses over time. In accordance with these embodiments, antibodies generated against SEQ ID NO:2 and/or SEQ ID NO:3 of SEMA7A can be used before, during or after other treatments including surgical tumor removal. In addition, one or more follow-up treatment with antibodies generated against SEQ ID NO:2 and/or SEQ ID NO:3 of SEMA7A can be used to reduce or eliminate presence of cancer stem cells. In some embodiments, in order to penetrate into a tumor cell population, cell penetrating agents can be attached to the one or more antibodies to improve efficacy, as needed. In some embodiments, conventional treatments can be used to eliminate the bulk of a tumor (e.g., radiation, chemotherapy, surgery, or other method) at the same time or after exposure of a subject to antibodies against SEQ ID NO:2 and/or SEQ ID NO:3 of SEMA7A followed by or exposed to at least one treatment of antibodies against SEQ ID NO:2 and/or SEQ ID NO:3 of SEMA7A to target cancer stem cells. In yet other embodiments, cell survival pathway inhibitors can be used to supplement these treatments before, during or after these antibody treatments (e.g., anti-BCL-2; anti-P13K inhibitors).

In certain embodiments, a dosing regimen described herein can include administering a SEMA7A antibody or monoclonal antibody disclosed herein to a subject as described herein as a direct injection (e.g., into a tumor such as a solid tumor), systemically, topically, intranasally, subcutaneously, in a bolus or in slow-release such as microparticles or other slow-release form to a subject. In other embodiments, a dosing regimen can include, but is not limited to, administering an antibody systemically (e.g., using parenteral administration). In certain embodiments, a dosing regimen can further include, but is not limited to, administering additional active agents (as described herein). In accordance with these embodiments, certain routes of administration suitable for delivering an SEMA7A antibody composition are described in more detail below.

In other embodiments, one or more peptides disclosed herein can be of use alone as the peptide or further be conjugated, carrier protein, or part of a fusion polypeptide (e.g., having a protein transduction domain such as tat or a fusion polypeptide having an Fc or other molecule, etc.). In accordance with these embodiments, these peptides or conjugates or fusion polypeptides thereof can be part of a composition or pharmaceutical composition contemplated herein. In other embodiments, these compositions can be pharmaceutical compositions and can be introduced to a subject to reduce the risk of onset (e.g., all or part of a tumor vaccine composition) and/or treat a health condition having SEMA7A expression or over expression. In certain embodiments, one or more antibodies directed to bind peptides having at least 75% homology to SEQ ID NO: 2 or SEQ. ID NO:3 are contemplated to be part of these pharmaceutical compositions for treating a subject having cancer or other health condition having adverse SEMA7A involvement. Any method for introducing or administering a peptide or peptide-containing composition to a subject in need thereof is contemplated such as orally, subcutaneously, intravenously, by bolus, by slow-release formulation, intranasally or combination administration thereof.

In other embodiments, compositions, and methods for inhibiting SEMA7A expression and/or activity disclosed herein can be used to reduce or prevent expansion, transformation, migration, stemness, metastasis and/or maturation of cancer stem cells found within a tumor or remaining after standard anti-tumor therapies. In certain embodiments, cancer stem cells include any cancer stem cell of any origin. In other embodiments, the cancer stem cells include any cancer stem cell resistant to radiation, chemotherapy, surgical removal, or other anti-cancer treatment wherein compositions and methods disclosed herein can be used to reduce or prevent cancer stem cell activities and survival to fully treat a subject in need of such a treatment. In other embodiments, cancer stem cells disclosed herein can include, or does include mammary cells and/or cells of mammary tissues (e.g., epithelial cells, MECs) or other cancer stem cells. In another embodiment, monoclonal antibody or polyclonal antibody compositions and methods disclosed herein can be used to inhibit metastasis of cancer stem cells, treat a tumor and/or reduce anoikis resistance in a subject. In certain embodiments, the subject is a female subject having PPBC. In other embodiments, antibodies against SEMA7A can be used to reduce or prevent expansion, migration, metastasis and/or maturation of cancer stem cells. In some embodiments, compositions and methods disclosed herein can be used to reduce expression, maturation, transformation, stemness, metastasis, and/or activity of SEMA7A in normal cells in order to treat a subject. In other embodiments, compositions, and methods for inhibiting SEMA7A expression can be used to reduce occurrence and/or expansion of drug resistant tumor cells such as stem cells or other tumor cells.

In certain embodiments, compositions, and methods for inhibiting SEMA7A expression and/or activity disclosed herein can be used before, during or after standard anti-tumor therapies. In accordance with these embodiments, any agent capable of inhibiting SEMA7A expression and/or activity can be used to reduce or eliminate cancer stem cells and/or reduce or eliminate development of drug resistance cancer cells and/or reduce cellular transformation of normal to a cancerous cell type in a subject. In some embodiments, compositions and methods disclosed herein can be used during and/or after standard anti-cancer therapies. In other embodiments, compositions, and methods for inhibiting SEMA7A expression and/or activity can be used following radiation therapy, chemotherapy, surgery or other tumor reducing treatment where agents capable of inhibiting SEMA7A (e.g., antibodies, chemical agents, snRNAs etc.) can be used to treat the remaining tumor cells including cancer stem cells to reduce or eliminate tumor cells including cancer stem cells or metastasis of these cells. In certain embodiments, the subject has breast cancer. In some embodiments, the breast cancer is PPBC breast cancers. In accordance with these embodiments, a subject having PPBC breast cancer has had or is undergoing postpartum mammary gland involution and can be treated as disclosed herein targeting cancer stem cells before, during or after standard treatments of care.

In other embodiments, isolated or synthetic SEMA7A antibodies or monoclonal antibodies or fragments thereof described herein can be used to treat a subject having cancer such as breast cancer. Some SEMA7A antibodies of use to treat cancer and/or reduce or eliminate cancer stem cells can include, but are not limited to, antibodies that bind to an epitope on a polypeptide having an amino acid sequence represented by amino acids 381-392 of SEQ ID NO: 1 or isomer thereof without binding to amino acids 371-380 and/or 393-441 of SEQ ID NO: 1 or isomer thereof or biologically active fragment thereof. In other embodiments, isolated or synthetic SEMA7A antibodies or monoclonal antibodies or fragments thereof disclosed herein can bind to an epitope on a polypeptide having an amino acid sequence represented by amino acids 381-392 of SEQ ID NO: 1 or isomer thereof without binding to more than five amino acids within amino acids 371-380 and/or 393-441 of SEQ ID NO: 1 and can be used in a pharmaceutical composition of use to inhibit SEMA7A expression or activity. Alternatively, SEMA7A antibodies of use to treat cancer, reduce metastasis or migration of tumor cells and/or reduce or eliminate cancer stem cells can include, but are not limited to, antibodies that bind preferably to an epitope on a polypeptide having an amino acid sequence represented by amino acids 381-392 of SEQ ID NO: 1 or isomer thereof with reduced binding to or minimal binding to amino acids 371-380 and/or 393-441 of SEQ ID NO: 1 and can be used in a pharmaceutical composition of use to inhibit SEMA7A expression or activity (e.g. pro-tumor, pro-metastasis, pro-cancer stem cell activities for example). In some embodiments, compositions disclosed herein can be used to reduce anoikis resistance and/or reduce activation of α6 and/or ß1-integrins and/or activation of phosphorylated AKT (pAKT) promoted by SEMA7A induction in a subject in need thereof in to reduce or eliminate cancer stem cell expansion and/or metastasis or other activity.

Isolated anti-SEMA7A Antibodies

In some embodiments, an isolated or synthetic SEMA7A antibody or monoclonal antibody or fragment thereof is provided. As used herein, the terms “SEMA7A antibody” or “antibody” can refer to an antibody capable of binding to and blocking expression of or activities of SEMA7A. In certain embodiments, SEMA7A polypeptides can include an amino acid sequence having 75% or 85% or more (e.g., 85%, 86%, 87%, 88%, 89%, 90%, 91%, 92%, 93%, 94%, 95%, 96%, 97%, 98%, 99%, or 100%) sequence identity to SEQ ID NO: 2 or 3 or isomeric equivalent thereof or amino acids 381-392 of SEQ ID NO:1; or in certain cases, antibodies generated against amino acids 371-441 of SEQ ID NO: 1 are contemplated of use to diagnose cancer and intervene as needed to treat cancer in the subject. In some embodiments, SEMA7A polypeptides of use herein can be conjugated to a carrier protein such as BSA or KLH or other carrier and can further include a linker (e.g., 20 amino acid or less, 3-mer, 4-mer up to 20-mer) that separates the carrier from the peptide. In certain embodiments, SEMA7A polypeptides can include an amino acid sequence 85% or more, up to 100% identical in sequence identity to SEQ ID NO: 2 or 3 or amino acids 381-392 of SEQ ID NO: 1 conjugated to a carrier with an amino acid linker between for use in generating one or more monoclonal antibodies. These monoclonal antibodies are used in pharmaceutical compositions to diagnose, prognose or treat SEMA7A expressing or overexpressing health conditions in a subject (e.g., cancer, such as breast cancer). In certain embodiments, these monoclonal antibodies are used to kill cancer cells including cancer stem cells in a subject.

In other embodiments, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof. In other embodiments amino acids 381-392 of SEMA7A are included as part of a longer polypeptide such as a 12mer, 14mer, 16mer, 18mer, or longer or other appropriate length polypeptide or a fragment thereof such as a 6mer or 8mer of this selected region. In certain embodiments, a polypeptide shorter than 40 amino acids in length is contemplated. In accordance with these embodiments, one or more of these polypeptides can be used to raise polyclonal and/or monoclonal antibodies of use herein.

In other embodiments, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof without binding to amino acids 371-380 and/or 393-441 of SEQ ID NO: 1 or isomer thereof. In other embodiments, isolated or synthetic SEMA7A antibodies or monoclonal antibodies or fragments thereof described herein can specifically bind to an epitope on a polypeptide having an amino acid sequence represented by amino acids 381-392 of SEQ ID NO: 1 or isomer thereof without binding to more than five amino acids of amino acids 371-380 and/or five amino acids of amino acids 393-441 of SEQ ID NO: 1. In certain embodiments, SEMA7A antibodies or monoclonal antibodies specifically bind to the polypeptide having an amino acid sequence represented by amino acids 381-392 of SEQ ID NO: 1 (ADRHPEVAQR SEQ ID NO:2; conjugated polypeptide with 3-mer linker: CGGADRHPEVAQR SEQ ID NO:3) with high affinity. In some embodiments, antibodies binding this epitope have superior activity (e.g. tumor cell killing) compared to commercially available antibodies against amino acids 1-100 of SEQ ID NO: 1 or amino acids 371-441 of SEQ ID NO: 1, for example.

In other embodiments, isolated or synthetic SEMA7A antibodies or monoclonal antibodies or fragments thereof described herein can specifically bind to an epitope unique to the human SEMA7A protein wherein any human isomer is contemplated. In other embodiments, isolated or synthetic SEMA7A antibodies or monoclonal antibodies or fragments thereof described herein cannot bind to a non-human SEMA7A protein.

A typical antibody molecule includes a heavy chain variable region (V_{H}) and a light chain variable region (V_{L}), which are usually included in antigen binding. The V_{H} and V_{L} regions can be further subdivided into regions of hypervariability, also known as “complementarity determining regions” (“CDR”), interspersed with regions that are more conserved, which are referred to as “framework regions” (“FR”). Each V_{H} and V_{L} is typically composed of three CDRs and four FRs, arranged from amino-terminus to carboxyl-terminus in the following order: FR1, CDR1, FR2, CDR2, FR3, CDR3, FR4. The extent of the framework region and CDRs can be precisely identified using methodology known in the art.

In some embodiments, provided herein are representative SEMA7A antibodies: monoclonal SEMA7A antibody clone H1 and monoclonal SEMA7A antibody clone H7—both for binding SEMA7A. In certain embodiments, antibodies can include a Vu region having 85% or more (e.g., 90%, 95%, 98%, 99% or more) sequence identity to the V_{H} region of monoclonal SEMA7A antibody clone H1 or monoclonal SEMA7A antibody clone H7. In certain embodiments, antibodies can include a V_{L} region having 85% or more (e.g., 90%, 95%, 98%, 99% or more) sequence identity to the V_{L} region of monoclonal SEMA7A antibody clone H1 or monoclonal SEMA7A antibody clone H7.

In certain embodiments, an isolated SEMA7A antibody can be a full-length antibody, which contains two heavy chains and two light chains, each including a variable domain and a constant domain. In certain embodiments, an isolated SEMA7 antibody can be an antigen binding fragment of a full-length antibody capable of binding SEMA7A and of use to treat cancer or other health conditions. In accordance with these embodiments, an SEMA7A antibody or antigen binding fragment thereof described herein can have binding fragments encompassed within the term “antigen-binding fragment” of a full length antibody can include (i) a Fab fragment, a monovalent fragment consisting of the V_{L}, V_{H}, C_{L}, and C_{H}1 domains; (ii) a F(ab′)₂ fragment, a bivalent fragment including two Fab fragments linked by a disulfide bridge at the hinge region; (iii) a Fd fragment consisting of the V_{H} and CHI domains; (iv) a Fv fragment consisting of the V_{L} and V_{H} domains of a single arm of an antibody, (v) a dAb fragment, which consists of a Vu domain; and (vi) an isolated complementarity determining region (CDR) that retains functionality. In accordance with these embodiments, a SEMA7A antibody disclosed herein can have two domains of the Fv fragment wherein, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv).

In some embodiments, isolated SEMA7A antibodies disclosed herein can be a single chain antibody (scFv). In accordance with some embodiments herein, a scFv antibody can be a V_{H} fragment and a V_{L} fragment, which can be linked via a flexible peptide linker. In some embodiments, a scFv antibody herein can be in the V_{H}→V_{L} orientation (from N-terminus to C-terminus). In some embodiments, a scFv antibody can be in the V_{L}→V_{H} orientation (from N-terminus to C-terminus).

### Preparation and Isolation of Antibodies

In certain embodiments, antibodies capable of binding to SEMA7A as described herein can be made by any method known in the art. See, for example, Harlow and Lane, (1998) Antibodies: A Laboratory Manual, Cold Spring Harbor Laboratory, New York. In particular embodiments, monoclonal antibodies herein can be generated using a conventional hybridoma technology and/or by isolating from an antibody library (e.g., obtained from antisera of an immunized animal). Isolated antibodies can be screened for a specific binding affinity to SEMA7A (and not to other targets). Once antibodies generated with high binding affinity for SEQ ID NO:2 or 3 or isomer thereof or antibodies generated with high binding affinity of conjugated polypeptides thereof are identified, the SEMA7A antibodies can be sequenced and recombinantly expressed, and then administered in an in vivo, in situ, or in vitro setting to analyze the ability of the antibodies to and block SEMA7A and its activities. In certain embodiments, these antibodies bind with high affinity to SEMA7A in purified form, in a sample such as a tumor or blood sample. In some embodiments, these antibodies are prepared as mammalian or humanized antibodies.

### Generating Antibodies

### Hybridoma Technology

In some embodiments, antibodies specific to a target antigen (e.g., SEMA7A SEQ ID NO:2 or 3 or isomer thereof) can be made by conventional hybridoma technology. In some embodiments, the full-length target antigen or a fragment thereof, optionally coupled to a carrier protein such as KLH, can be used to immunize a host animal (e.g., a mouse or a canine) for generating antibodies binding to that antigen. In some embodiments, the route and schedule of immunization of the host animal are generally in keeping with established and conventional techniques for antibody stimulation and production, as further described herein. In other embodiments, it is contemplated that any mammalian subject can be manipulated to serve as the basis for production of mammalian, including mouse hybridoma cell lines. In some embodiments, the host animal can be inoculated intraperitoneally, intramuscularly, orally, subcutaneously, intraplantar, and/or intradermally with an amount of immunogen, including as described herein. In some embodiments, immunization of a host animal with a target antigen SEQ ID NO:2 or 3 or isomer thereof, or a fragment containing the target amino acid sequence conjugated to a protein that is immunogenic in the species to be immunized, e.g., keyhole limpet hemocyanin (KLH), serum albumin (e.g., bovine serum albumin (BSA)), bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies for use herein.

In some embodiments, hybridomas can be prepared from the lymphocytes and immortalized myeloma cells using the general somatic cell hybridization techniques known in the art. Available myeloma lines can be used. In some embodiments, the technique that can be used herein can include fusing myeloma cells and lymphoid cells to produce hybridomas. In some embodiments, cell fusion technique, EBV immortalized B cells can be used to produce the SEMA7A monoclonal antibodies described herein. In accordance with these embodiments, hybridomas can be expanded and subcloned, and supernatants can be assayed for anti-immunogen activity by conventional immunoassay procedures (e.g., radioimmunoassay, enzyme immunoassay, or fluorescence immunoassay).

In certain embodiments, hybridomas that can be used as source of antibodies herein can encompass all derivatives, progeny cells of the parent hybridomas that produce monoclonal antibodies capable of binding SEMA7A or the epitope designated as SEQ ID NO:2 or 3 or isomer thereof of SEMA7A. In some embodiments, hybridomas herein that can produce such antibodies can be grown in vitro or in vivo using known procedures.

In some embodiments, monoclonal antibodies can be isolated from the culture media or body fluids, by conventional immunoglobulin purification procedures such as ammonium sulfate precipitation, gel electrophoresis, dialysis, chromatography, and ultrafiltration, and the like. In some embodiments, undesired activity, if present, can be removed by, for example but not limited to, running the preparation over adsorbents made of the immunogen attached to a solid phase and eluting or releasing the desired antibodies off the immunogen.

Isolation from an Antibody Library

In certain embodiments, antibodies capable of binding and interfering with the target antigens as described herein can be isolated from an antibody library generated by immunizing an animal and collecting antisera from the animal. This generates a library of unique antibodies or antibody components that can be used to identify antibodies that bind to a specific target antigen (e.g., SEMA7A, SEQ ID NO:2 or 3 or isomer thereof of SEMA7A) following routine selection processes as known in the art. In some embodiments, an antibody library can be probed with the target antigen or a fragment thereof and members of the library that are capable of binding to the target antigen can be isolated, typically by retention on a support. In some embodiments, a screening process herein can be performed by multiple rounds (e.g., including both positive and negative selections) to enrich the pool of antibodies capable of binding to the target antigen. In other embodiments, individual clones of the enriched pool can then be isolated and further characterized to identify those having desired binding activity and biological activity. In some embodiments, sequences of the heavy chain and light chain variable domains can also be determined via conventional methodology. There are a number of routine methods known in the art to identify and isolate antibodies capable of binding to the target antigens described herein, including phage display, yeast display, ribosomal display, or mammalian display technology.

In certain embodiments, phage displays herein can use a covalent linkage to bind the protein (e.g., antibody) component to a bacteriophage coat protein. In some embodiments, the linkage can result from translation of a nucleic acid encoding the antibody component fused to the coat protein. In some embodiments, the linkage can include a flexible peptide linker, a protease site, or an amino acid incorporated as a result of suppression of a stop codon. In some embodiments, a bacteriophage displaying the protein component can be grown and harvested using standard phage preparatory methods, (e.g., PEG precipitation from growth media). In some embodiments, after selection of individual display phages, the nucleic acid encoding the selected protein components can be isolated from cells infected with the selected phages or from the phage themselves, after amplification. In some embodiments, individual colonies or plaques can be selected, and then the nucleic acid can be isolated and sequenced.

### Identifying Antibodies or Antigen Binding Proteins Having a Specific Binding Affinity

In certain methods and procedures described above, antibodies capable of binding to SEMA7A can be prepared and isolated. In certain embodiments, after antibodies are isolated from the procedures described above for binding to the target antigen, each isolated library member can be tested for its ability to bind to a non-target molecule to evaluate its binding specificity. Examples of non-target molecules include, but are not limited to, streptavidin on magnetic beads, blocking agents such as bovine serum albumin, non-fat bovine milk, soy protein, any capturing or target immobilizing monoclonal antibody, or non-transfected cells which do not express the target. In some embodiments, a high-throughput ELISA screen can be used to obtain the data. In some embodiments, an ELISA screen can also be used to obtain quantitative data for binding of each library member to the target as well as for cross species reactivity to related targets or subunits of the target antigen and also under different condition such as pH 6 or pH 7.5. In accordance with some embodiments herein, non-target and target binding data can be compared (e.g., using a computer and software) to identify library members that specifically bind to the target.

In accordance with some embodiments herein, after selecting candidate library members that bind to a target, each candidate library member can be further analyzed, e.g., to further characterize its binding properties for the target, e.g, human SEMA7A, SEQ ID NO:2 or 3 or isomer thereof of SEMA7A. In some embodiments, each candidate library member can be subjected to one or more secondary screening assays. In some embodiments, the assay can be for a binding property, a catalytic property, an inhibitory property, a physiological property (e.g., cytotoxicity, renal clearance, or immunogenicity), a structural property (e.g., stability, conformation, oligomerization state) or another functional property. In some embodiments, the same assay can be used repeatedly, but with varying conditions, e.g., to determine pH, ionic, or thermal sensitivities.

In some embodiments, binding proteins can be evaluated using an ELISA assay. In accordance with some embodiments herein, each protein can be contacted to a microtiter plate whose bottom surface has been coated with the target, e.g., a limiting amount of the target. In accordance with some embodiments herein, plates can be washed with buffer to remove non-specifically bound polypeptides. In accordance with these embodiments herein, the amount of the binding protein bound to the target on the plate can be determined by probing the plate with an antibody that can recognize the binding protein, e.g., a tag or constant portion of the binding protein. In accordance with some embodiments herein, the antibody can be linked to a detection system (e.g., an enzyme such as alkaline phosphatase or horse radish peroxidase (HRP) which produces a colorimetric product when appropriate substrates are provided).

In some embodiments, binding proteins can be screened for ability to bind to cells which transiently or stably express and display the target of interest on the cell surface. In some embodiments, SEMA7A binding proteins herein can be fluorescently labeled and binding to SEMA7A in the presence or absence of antagonistic antibody can be detected by a change in fluorescence intensity using flow cytometry e.g., a FACS machine.

In certain embodiments, at any time during the antibody generation process, one or more positive binding antibodies to SEMA7A can be identified. In certain embodiments, certain methods further include sequencing the positive antibodies and expressing the sequence in a recombinant microorganism generated by any method known in the art.

Polynucleotides, vectors, and host cells can be used to prepare an SEMA7A antibody using recombinant technology, as exemplified herein. In certain embodiments, nucleic acids encoding the heavy and light chain of an SEMA7A antibody as described herein can be cloned into one expression vector, each nucleotide sequence being in operable linkage to a suitable promoter. In some embodiments, each of the nucleotide sequences encoding the heavy chain and light chain is in operable linkage to a distinct promoter. In some embodiments, nucleotide sequences encoding the heavy chain and the light chain can be in operable linkage with a single promoter, such that both heavy and light chains are expressed from the same promoter. In some embodiments, when necessary, an internal ribosomal entry site (IRES) can be inserted between the heavy chain and light chain encoding sequences.

In certain embodiments, the expression and/or production of the antibodies in the host cell can be promoted by expressing a leader peptide ahead of the variable region of the heavy or light chain of the antibody. This leader peptide can be optimized for expression in a certain host cell (e.g., E. coli). One representative sequence that can be used as a leader peptide includes, but is not limited to, a 10-mer or fragment thereof or 12mer, or 14mer or 16mer or 18mer or other larger fragment containing a polypeptide. In some embodiments, a leader sequence can be relatively short such as less than 5 amino acids in length. Leader sequences that are the same length, shorter and longer are known in the art and contemplated of use herein.

In some embodiments, genetically engineered antibodies such as single-chain antibodies can be produced via, e.g., conventional recombinant technology or any methods known in the art. In some embodiments, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA can be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. The DNA can then be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some embodiments, genetically engineered antibodies, such as chimeric or hybrid antibodies; can be prepared that have the binding specificity of a target antigen.

In some embodiments, a single-chain antibody herein can be prepared via recombinant technology by linking a nucleotide sequence coding for a heavy chain variable region and a nucleotide sequence coding for a light chain variable region. In some embodiments, a flexible linker is incorporated between the two variable regions. In some embodiments, techniques described for the production of single chain antibodies can be adapted to produce a phage or yeast scFv library and scFv clones specific to SEMA7A can be identified from the library following routine procedures. In some embodiments, positive clones can be subjected to further screening to identify those that bind to SEMA7A or only to SEMA7A or epitope thereof or SEQ ID NO:2 or 3 or isomer thereof of SEMA7A.

In some embodiments, one or more vectors (e.g., expression vectors) having nucleic acids encoding any of the antibodies herein can be introduced into suitable host cells for producing the antibodies. In some embodiments, host cells can be cultured under suitable conditions for expression of the antibody or any polypeptide chain thereof. In some embodiments, antibodies or polypeptide chains thereof can be recovered by the cultured cells (e.g., from the cells or the culture supernatant) via a conventional method, e.g., affinity purification. In some embodiments, polypeptide chains of the antibody herein can be incubated under suitable conditions, for a suitable period of time allowing for production of the antibody.

In some embodiments, methods for preparing an antibody described herein can include a recombinant expression vector that encodes both the heavy chain and the light chain of an SEMA7A antibody, as also described herein. In some embodiments, a recombinant expression vector can be introduced into a suitable host cell (e.g., a dhfr-CHO cell) by a conventional method, e.g., calcium phosphate-mediated transfection. In some embodiments, positive transformant host cells can be selected and cultured under suitable conditions allowing for the expression of the two polypeptide chains that form the antibody, which can be recovered from the cells or from the culture medium. In some embodiments, the two chains recovered from the host cells can be incubated under suitable conditions allowing for the formation of the antibody.

In certain embodiments, two recombinant expression vectors can be provided, one encoding the heavy chain of the SEMA7A antibody and the other encoding the light chain of the SEMA7A antibody. In some embodiments, both of the two recombinant expression vectors can be introduced into a suitable host cell (e.g., dhfr-CHO cell) by a conventional method, e.g., calcium phosphate-mediated transfection. In some embodiments, each of the expression vectors can be introduced into a suitable host cells. In some embodiments, positive transformants can be selected and cultured under suitable conditions allowing for the expression of the polypeptide chains of the antibody. In some embodiments, when the two expression vectors are introduced into the same host cells, the antibody produced therein can be recovered from the host cells or from the culture medium. In some embodiments, the polypeptide chains can be recovered from the host cells or from the culture medium and then incubated under suitable conditions allowing for formation of the antibody. In some embodiments, when the two expression vectors are introduced into different host cells, each of them can be recovered from the corresponding host cells or from the corresponding culture media. In some embodiments, two polypeptide chains can then be incubated under suitable conditions for formation of the antibody.

In certain embodiments, standard molecular biology techniques can be used to prepare the recombinant expression vector, transfect the host cells, select for transformants, culture the host cells and recovery of the antibodies from the culture medium. In some embodiments, some antibodies can be isolated by affinity chromatography with a Protein A or Protein G coupled matrix.

In additional embodiments, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, competition assays, gene fragment expression assays, and synthetic peptide-based assays. In some embodiments, epitope mapping can be used to determine the sequence, to which an antibody binds and the strength to which it binds a particular epitope.

### Therapeutic and Diagnostic Applications

In some embodiments, SEMA7A inhibition can reduce tumor growth and expansion, progression and metastasis. Cancers to be treated contemplated herein can include solid or non-solid tumors such as cancers of the blood. Solid tumors can include, but are not limited to, breast, pancreatic, brain, mesothelioma, lung, thyroid, stomach, liver, kidney, ovarian, prostate, breast or other solid tumor. Non-solid tumors include, but are not limited to, leukemias and other known non-solid tumors and metastasizing tumors. In accordance with these embodiments, use of one or more antibody or monoclonal antibody (e.g., against SEQ ID NO:2 or 3 or isomer thereof) disclosed herein can be used to treat a subject having cancer in order to reduce or prevent expansion, migration, shrink a tumor and/or metastasis of the cancer. In some embodiments, antibodies or monoclonal antibody treatments disclosed herein can be used to treat a subject daily, every other day, every three days, bi-weekly, every week, bi-monthly or other appropriate treatment regimen. In other embodiments, a subject can be treated when SEMA7A is detected in a blood or other sample which can include a single or multiple treatments with antibodies disclosed herein. In other embodiments, other markers can be measured besides SEMA7A in one or more samples such as a blood or tumor biopsy. In accordance with these embodiment, one or more samples such as a blood or tumor biopsy can be assessed for levels of one or more of alpha-6 integrin, FN, COX-2 and PD-L1 in order to tailor treatment of the subject to reduce, eliminate or prevent a condition such as cancer (e.g., breast cancer or PPBC etc.)

In other embodiments, one or more antibodies disclosed herein or any other SEMA7A antibody available can be used to determine prognosis of a subject having a health condition. In accordance with these embodiments, a SEMA7A antibody or an antibody or monoclonal antibody disclosed herein can be used to measure levels or presence of SEMA7A in a sample (e.g. a fluid sample) from a subject and prognosis of the subject can be determined. In some embodiments, a poor or positive prognosis can be assessed based on level of SEMA7A in the tissue, blood or other fluid sample of a subject having a heart attack or other condition such as cancer, chronic inflammation, or fibrosis. In accordance with these embodiments, a SEMA7A concentration in a blood sample of about 0.1 ng/ml, or about 0.5 ng/ml or about 1.0 ng/ml and above can indicate a poor prognosis and intervention should be undertaken or course of treatment modified or increased. In some embodiments, a level of about 1 ng/ml in a fluid sample (e.g. blood) of a subject corresponds to a poor prognosis in a subject for a health condition including, but not limited to, a subject having a heart attack, a subject having cancer or pre-malignant disease (e.g. benign breast disease, atypical hyperplasia or carcinoma in situ), a subject having an inflammatory condition, fibrosis or other health condition where SEMA7A is expressed or over-expressed and/or contributes to adverse health conditions in a subject. In some embodiments, where at risk levels of SEMATA are observed, a SEMA7A antibody or other inhibitor (e.g. monoclonal antibody) can be administered as a therapeutic intervention to prevent or reduce progression of the health condition. In certain embodiment, a SEMA7A polyclonal antibody or monoclonal antibody against SEQ ID NO:2 or 3 or isomer thereof is administered as a therapeutic intervention to prevent or reduce progression of the health condition such as cancer. In some embodiments, a candidate dose is an equivalent dose of about 5 to about 20 ug per dose in comparison to studies disclosed herein or a dose known in the art for inhibiting SEMA7A adverse effects, or a standard monoclonal antibody concentration known in the art.

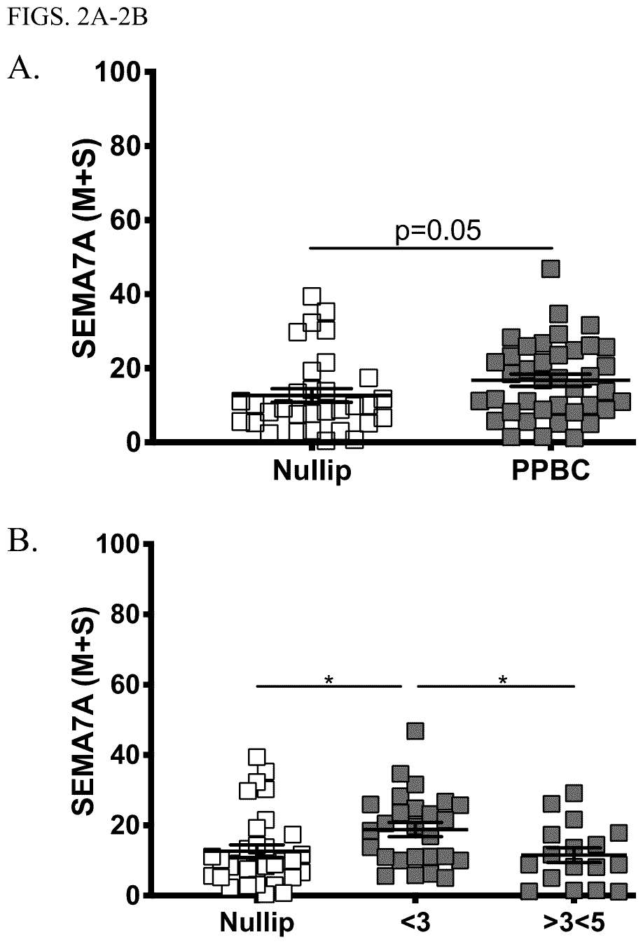

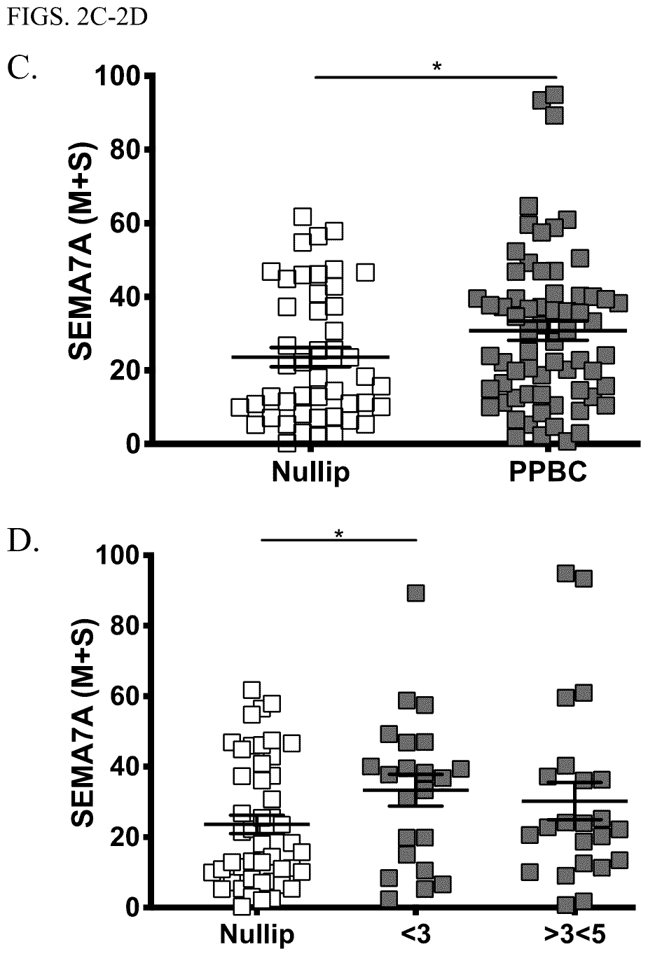

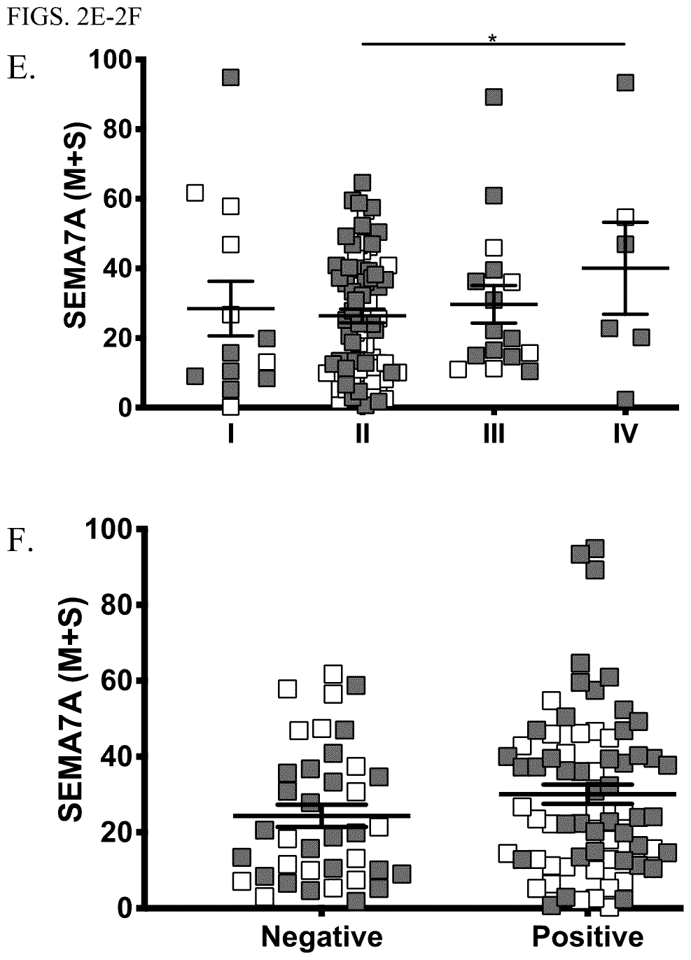

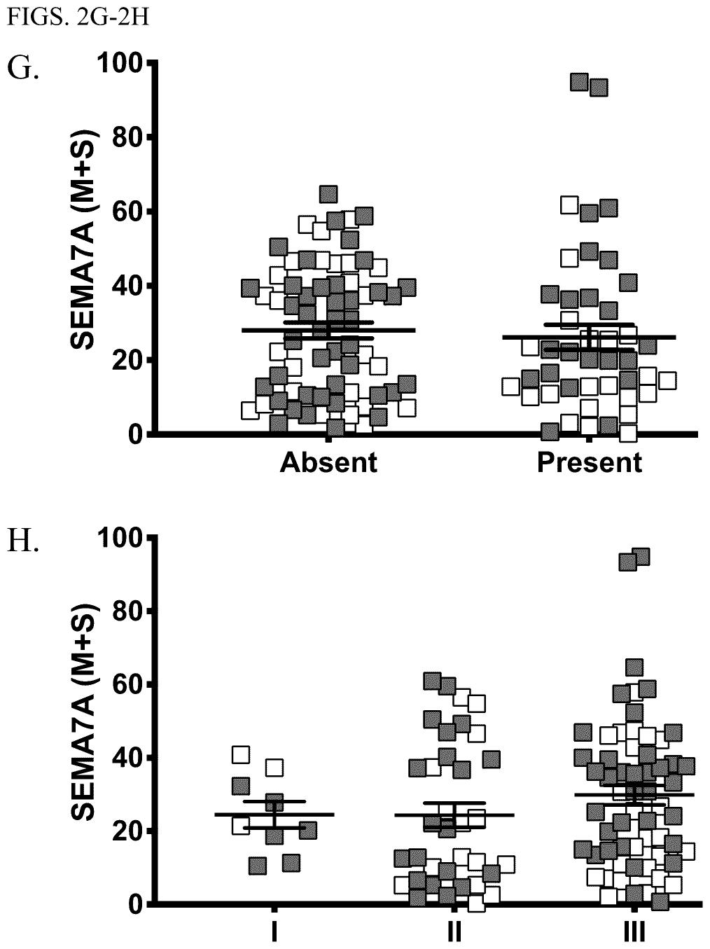

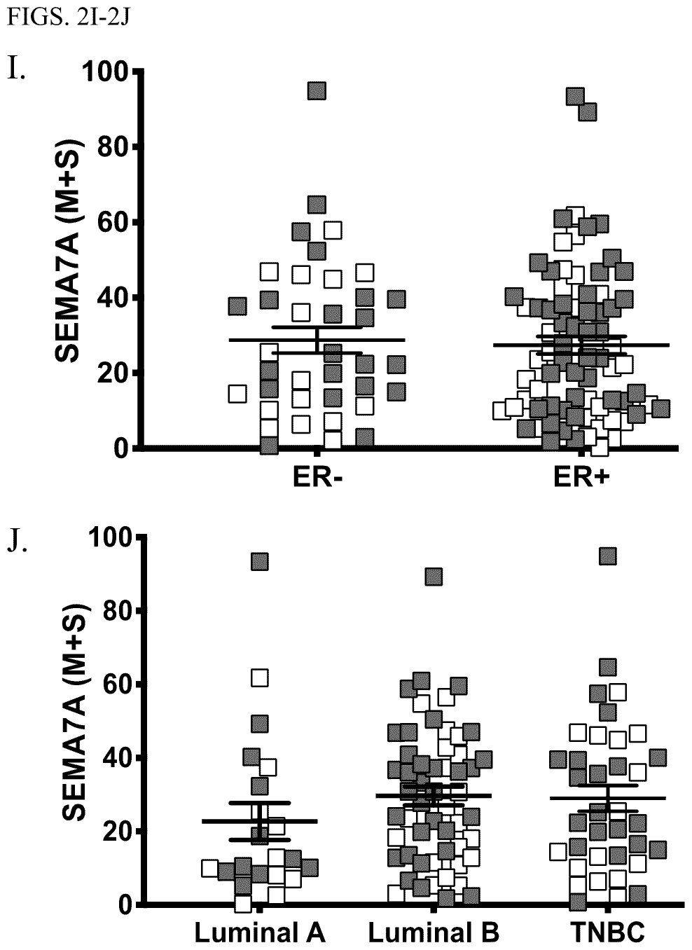

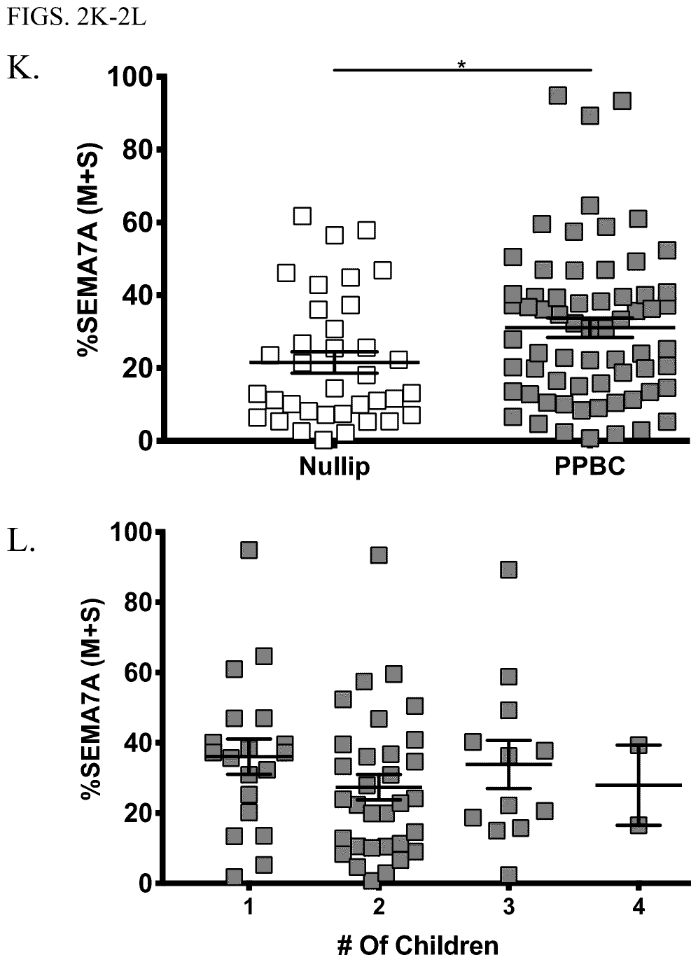

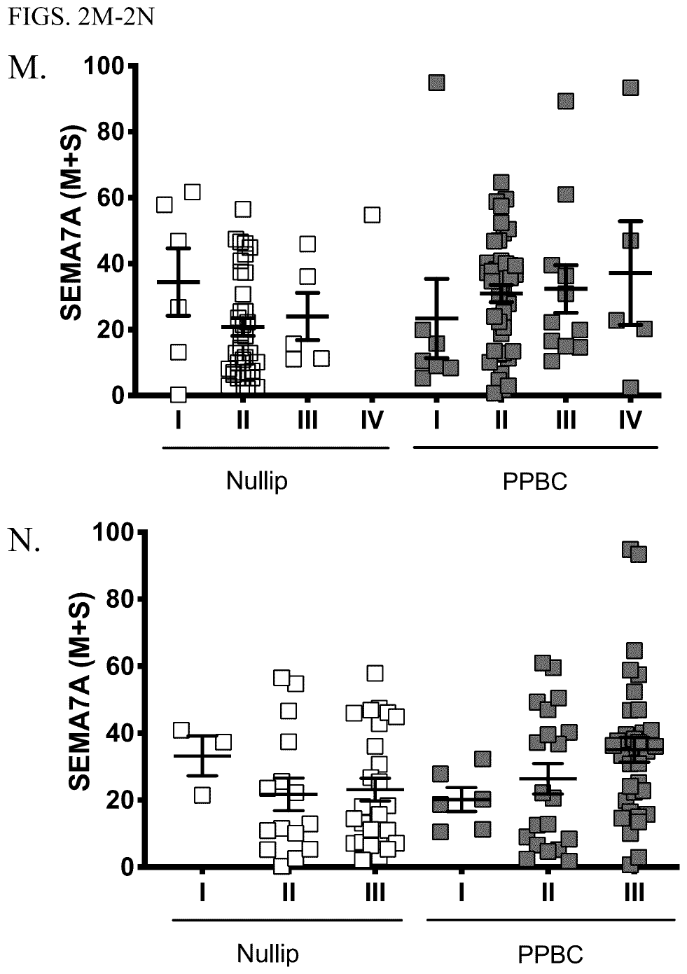

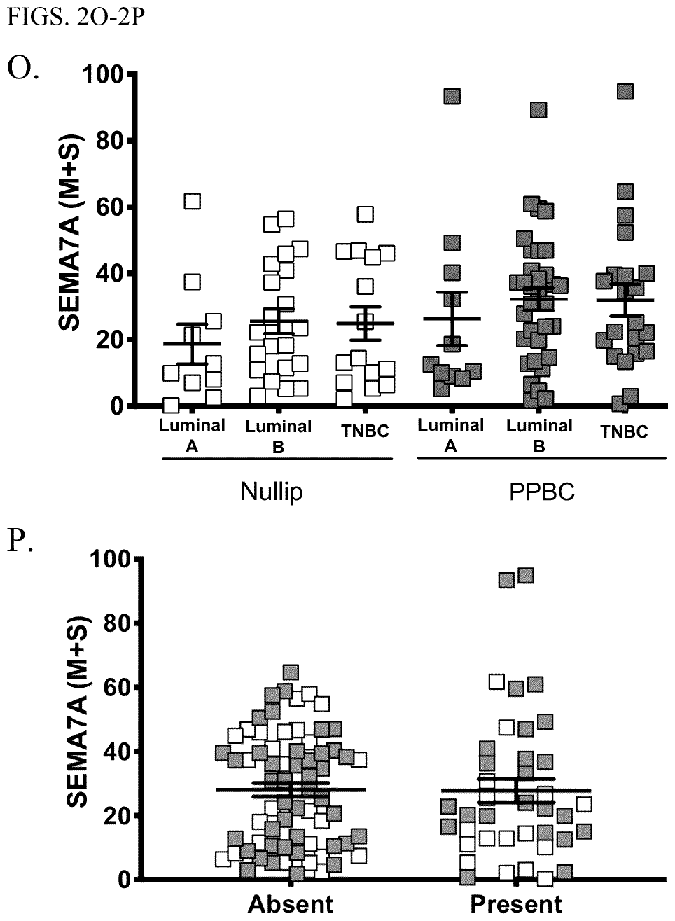

In another embodiment, antibodies can be raised against amino acids 371-441 of the SEMA7A protein sequence represented by SEQ ID NO: 1 or equivalent positions thereof. In accordance with these embodiments, these antibodies will recognize all isoforms of SEMA7A. In certain embodiments, these antibodies can be used to diagnose cancer in a subject. In certain embodiments, these antibodies can be used to diagnose breast cancer in a subject. In yet other embodiments, antibodies raised against amino acids spanning residues 371-441 of SEMA7A represented by SEQ ID NO: 1, or equivalent positions thereof can be used to assay samples from a subject in order to identify SEMA7A presence or concentrations in the one or more samples. In accordance with these embodiments, presence of SEMA7A in the one or more samples can be an indicator of poor prognosis in the subject. In other embodiments, presence of SEMA7A in the one or more samples diagnosis a breast cancer pa. expression was compared with multiple breast cancer clinical characteristics that are known prognostic indicators across all 113 cases. There were no differences seen in SEMA7A expression based on grade, stage, LN status, LVI, or biologic subtype when cases were analyzed as one combined group. Next, expression of SEMA7A was analyzed between the nulliparous and PPBC groups. First, it was observed that SEMA7A expression was significantly increased in the normal-adjacent tissues of the PPBC group compared with nulliparous and that this increase was most notable for those cases diagnosed within 3 years of last pregnancy and declined among cases that are further out from their last childbirth with significance lost by a diagnosis >3 to <5 years after birth (FIGS. 2A and 2B). Similarly, PPBCs had higher tumor SEMA7A expression than nulliparous; however, in the tumors, the level of SEMA7A expression remained higher even among cases with more time between last parturition and diagnosis, without a decrease as seen in the normal tissue, up to our 5-year current cut-off (FIGS. 2C and 2D).

In certain embodiments, antibodies (e.g. monoclonal antibodies) disclosed herein directed to bind SEMA7A can be used to treat health conditions associated with expression or over-expression of SEMA7A or SEMA7A activity. In accordance with these embodiments, the health condition to be treated includes, but is not limited to, health conditions concerning neurological development, neurological conditions, wound healing, bone homeostasis conditions, angiogenesis issues, pro-inflammatory cytokine release and regulation-related conditions, dendritic cell-related adverse effects on adhesion and motility, fibrosis-related conditions and effects on anti-inflammatory IL-10 release effects as well as effects on tumor metastasis and progression where inhibition of SEMA7A expression and/or activities treats the condition. In certain embodiments, antibodies disclosed herein can be used to treat one or more of these conditions to alleviate the condition.

In some embodiments, cancers can be treated with SEMA7A antibodies or other inhibitory molecules or technologies disclosed herein to reduce tumor progression, reduce or eliminate cancer stem cells or cancer stem cell metastasis, reduce or inhibit metastasis of other tumor cells, shrink tumors, induce tumor cell death, reduce tumor expansion, reduce lymphatic invasion and/or reduce lymphogenous metastasis (e.g. reduce infiltration or metastasis of tumor cells into lymph nodes) In other embodiments, antibodies disclosed herein can be used to reduce recruitment of suppressive immune cells such as macrophages and to reduce fibrillar collagen deposition. In yet other embodiments, SEMA7A presence or levels can be detected in a tumor in order to assess SEMA7A involvement in pro-tumor effects. In accordance with these embodiments, if SEMA7A is present or the level has reached a predetermined threshold (e.g. 1 ng/ml), the tumor can be treated with one or more SEMA7A inhibitors to reduce tumor expansion, shrink tumor size and/or reduce tumor metastasis. These treatments can be used alone or in combination with other treatments.

In other embodiments, inhibitors of SEMA7A expression or activities can include any SEMA7A inhibitor. In certain embodiments, inhibitors of SEMA7A expression or activities can include, but are not limited to, antibodies, anti-sense RNA, siRNA, an agent that targets SEMA7A or use of any editing technology such as Crispr or other gene editing technology capable of genetically manipulating SEMA7A to edit SEMA7A by completely removing it or editing it to reduce its pro-tumor activities, for example. It is contemplated that any of these methods and systems can be used to treat a subject having cancer with SEMA7A involvement.

In certain embodiments, a nucleic acid molecule disclosed herein can be used to modulate gene expression of SEMA7A in a targeted cell. As used herein, the term “genetically modified” refers to manipulation of a cell genome using genetic engineering techniques. Non-limiting examples of genetic engineering techniques that can be used to modulate gene expression of SEMA7A in a target cell can include chemical mutagenesis, x-ray mutagenesis, recombinant DNA techniques, virus-mediated delivery of DNA, and gene editing. Some examples of gene editing methods include, but are not limited to, CRISPRs, TALENs and Zinc Finger Nucleases.

### Pharmaceutical Compositions

In certain embodiments, pharmaceutical compositions are provided herein. The pharmaceutical composition can contain a pharmaceutically acceptable carrier combined with an SEMA7A antibody as described herein. Pharmaceutically acceptable excipients (carriers) are well known in the art.

In certain embodiments, the pharmaceutical compositions can further include a non-specific innate immune response stimulator mixture or composition or other immunomodulatory agent for enhancing an immune response. In accordance with these embodiments, the non-specific innate immune response stimulator can elicit both a cell-mediated immune response and a humoral immune response.

In other embodiments, pharmaceutical compositions described herein can further include an anti-microbial agent, a chemotherapeutic agent, and/or other anti-cancer therapeutic or antibody. In accordance with these embodiments, the anti-microbial agent can, in an example, be an anti-viral, bactericidal agent, anti-fungal, or anti-bacterial agent. For example, the anti-microbial agent can be an anti-bacterial agent (antibiotic) such as doxycycline or other antibiotic such as a general antibiotic. In other embodiments, targeted anti-cancer agents such as those that target receptor tyrosine kinases, hormone receptors, anti-apoptotic mechanisms, cell cycle inhibitors, metabolism, inflammation, statins and additional agents can in advance of or at the time of or after administering SEMA7A antibodies or inhibitors disclosed herein.

Pharmaceutically acceptable carriers or excipients suitable for the compositions described herein are well known to one of skill in the art of use for preserving and delivering antibodies or antibody fragments to any mammalian subject including humans and other mammals. It is contemplated that any pharmaceutically acceptable agent can be used in compositions disclosed herein.

In certain embodiments, the pharmaceutical compositions to be used in the present methods can include pharmaceutically acceptable carriers, excipients, or stabilizers in the form of lyophilized formulations or aqueous solutions. In some embodiments, acceptable carriers, excipients, or stabilizers are nontoxic to recipients at the dosages and concentrations used, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some embodiments, the pharmaceutical composition described herein can have liposomes containing the antibodies (or the encoding nucleic acids). In some embodiments, liposomes for use herein can be generated by the reverse phase evaporation method with a lipid composition having phosphatidylcholine, cholesterol and PEG-derivatized phosphatidylethanolamine (PEG-PE). In some embodiments, liposomes for use herein can be extruded through filters of defined pore size to yield liposomes with the desired diameter.

In some embodiments, antibodies, or the encoding nucleic acid(s) herein, can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other embodiments, the pharmaceutical compositions described herein can be formulated in sustained-release format. In some embodiments, pharmaceutical compositions herein to be used for in vivo administration must be sterile. In some embodiments, this can be readily accomplished by, for example, filtration through sterile filtration membranes. In some embodiments, therapeutic antibody compositions can be placed into a container having a sterile access port, for example, an intravenous solution bag or vial having a stopper pierceable by a hypodermic injection needle.

In certain embodiments, pharmaceutical compositions described herein can be in unit dosage forms such as tablets, pills, capsules, powders, granules, solutions or suspensions, or suppositories, for oral, parenteral, or rectal administration, or administration by inhalation or insufflation.

In some embodiments, emulsion compositions herein can be those prepared by mixing an antibody with Intralipid™ or the components thereof (soybean oil, egg phospholipids, glycerol and water).

In some embodiments, pharmaceutical compositions herein for inhalation or intranasal administration include solutions and suspensions in pharmaceutically acceptable, aqueous or organic solvents, or mixtures thereof, and powders. In some embodiments, liquid or solid compositions herein can contain suitable pharmaceutically acceptable excipients as set out above. In other embodiments, the compositions can be administered by the oral or nasal respiratory route for local or systemic effect.

In some embodiments, compositions can be in sterile pharmaceutically acceptable solvents can be nebulized by use of gases. In some embodiments, nebulized solutions herein can be breathed directly from the nebulizing device or the nebulizing device can be attached to a face mask, tent or intermittent positive pressure breathing machine. In some embodiments, solution, suspension or powder compositions herein can be administered, preferably orally or nasally, from devices which deliver the formulation in an appropriate manner.

In some embodiments, concentrations of antibodies disclosed herein can be a predetermined concentration or a standard concentration. In some embodiments, the can be in a concentration of about 1-1000 mg/ml, 1-500 mg/ml, 1-250 mg/ml, 1-200 mg/ml, 1-150 mg/ml, 1-100 mg/ml, 1-75 mg/ml, or 1-50 mg/ml, or 0.1 to 100 micrograms or other suitable concentration. In some embodiments, the antibody is formulated to a concentration of about 30 mg/ml. In some embodiments, the antibody is lyophilized. In some embodiments, the antibody is diluted in a suitable solution to a suitable concentration prior to administration (e.g., in a therapeutic application described below).

### Methods of Use—Therapeutic Applications

In certain embodiments, the cancer to be treated by compositions disclosed herein is a solid tumor cancer. In other embodiments, the solid tumor can be a head, neck, lung, breast, liver, or colon tumor. In some embodiments, administering compositions disclosed herein can be by direct injection into the tumor of the subject. In certain embodiments, the method can include systemic administration of the composition.

In certain embodiments, a subject having a targeted cancer can be identified by routine medical examination, e.g., laboratory tests, organ functional tests, CT scans, or ultrasounds. In some embodiments, the subject to be treated by the method described herein can have undergone or is subjecting to an anti-cancer therapy, for example, chemotherapy, radiotherapy, immunotherapy, or surgery.

In some embodiments, treatment efficacy for a target disease/disorder can be assessed by methods well-known in the art. In accordance with these embodiments, cancer progression or remission can be analyzed by any method known in the art in order to adjust treatment regimens as needed.

### Kits

In certain embodiments, kits are provided for storage, transport and use in treating or alleviating a target disease, such an immunosuppressed condition or cancer as described herein. In some embodiments, kits can include one or more containers. In other embodiments, kits disclosed herein contain at least one anti-SEMA7A antibody or composition thereof. In some embodiments, kits can include an anti-SEMA7A monoclonal antibody directed to bind SEQ ID NO:2 or 3 or biologically active fragment thereof.

In some embodiments, kits can include instructions for use in accordance with any of the methods described herein. In other embodiments, containers, delivery devices and/or instructions can be included and can contain a description of methods for administration of the SEMA7A antibody, and optionally, a second therapeutic agent, to treat, delay the onset, or alleviate a condition as those described herein. In other embodiments, kits can further include a description of selecting an individual suitable for treatment based on identifying whether that individual has the target disease, e.g., applying the diagnostic method as described herein. In still other embodiments, the instructions can have a description of administering an antibody to an individual at risk of the target disease. In certain embodiments, kits can contain one or more polypeptides of use to generate the antibodies disclosed herein.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of embodiments of the inventions.

### Example 1

In one exemplary method, peptide fragments of Semaphorin 7A (SEMA7A, referred to also as CD108, or Sema KI or Sema L) were generated. SEMA7A is a member of the Semaphorin family of axon guidance molecules and, when expressed on human erythrocytes, is the John Milton Hagen (JMH) blood group antigen. SEMA7A is an ˜80 kDa membrane-anchored glycoprotein that contains an RGD integrin interaction motif within its full-length amino acid sequence where a representative sequence is referenced below:

HUMAN SEMA7A
(SEQ ID NO: 1)
MTPPPPGRAAPSAPRARVPGPPARLGLPLRLRLLLLLWAAAASAQGHLRS
GPRIFAVWKGHVGQDRVDFGQTEPHTVLFHEPGSSSVWVGGRGKVYLFDF
PEGKNASVRTVNIGSTKGSCLDKRDCENYITLLERRSEGLLACGTNARHP
SCWNLVNGTVVPLGEMRGYAPFSPDENSLVLFEGDEVYSTIRKQEYNGKI
PRFRRIRGESELYTSDTVMQNPQFIKATIVHQDQAYDDKIYYFFREDNPD
KNPEAPLNVSRVAQLCRGDQGGESSLSVSKWNTFLKAMLVCSDAATNKNF
NRLQDVFLLPDPSGQWRDTRVYGVFSNPWNYSAVCVYSLGDIDKVFRTSS
LKGYHSSLPNPRPGKCLPDQQPIPTETFQVADRHPEVAQRVEPMGPLKTP
LFHSKYHYQKVAVHRMQASHGETFHVLYLTTDRGTIHKVVEPGEQEHSFA
FNIMEIQPFRRAAAIQTMSLDAERRKLYVSSQWEVSQVPLDLCEVYGGGC
HGCLMSRDPYCGWDQGRCISIYSSERSVLQSINPAEPHKECPNPKPDKAP
LQKVSLAPNSRYYLSCPMESRHATYSWRHKENVEQSCEPGHQSPNCILFI
ENLTAQQYGHYFCEAQEGSYFREAQHWQLLPEDGIMAEHLLGHACALAAS
LWLGVLPTLTLGLLVH

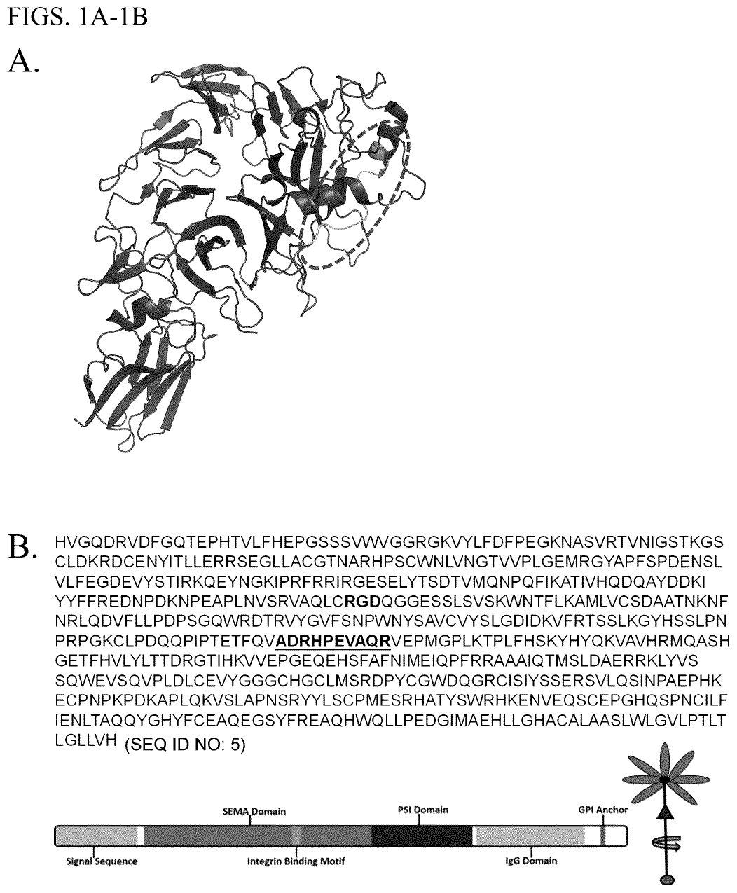

Using SEQ ID NO: 1 as a representative sequence of SEMA7A, a 10 amino acid peptide was identified of use in constructs and compositions disclosed herein. This peptide was identified having only exhibited sequence homology to human SEMA7A and no other known peptide. The peptide was synthesized and purified. The 10 amino acid SEMA7A peptide corresponds to amino acid residues 381-392 of the human full-length SEMA7A (SEQ ID NO: 1) and has an amino acid sequence of: ADRHPEVAQR (SEQ ID NO: 2). FIG. 1A illustrates a 2D structure of a human SEMA7A protein where the location of the selected 10 amino acid sequence used to design the peptides herein is indicated by a loop. FIG. 1B illustrates the context of the peptide within the domains of SEMA7A and its proximity to the beta-1 integrin binding site. In brief, state-of-the-art automated instrumentation for solid-phase peptide synthesis was used to synthesize the peptides of interest using 3-maleimido-benzoic acid-OSu (Boc)-chemistry. The resulting peptides were then subjected to peptide purification by reverse-phase HPLC followed by peptide identification by mass spectrometry (LC-MS). In addition to the 10 amino acid SEMA7A peptide, a peptide for later conjugation was also synthesized (e.g., CGGADRHPEVAQR; SEQ ID NO: 3). It is noted that other conjugated constructs or polypeptide fragments or polynucleotide related thereto can be of use in constructs and compositions for generating antibodies disclosed herein having at least 75% up to 100% homology to the 10-mer and of longer or shorter lengths such as a 5-mer or a 15-mer, 20-mer or other suitable size.

### Example 2

In another exemplary method, the 10 amino acid SEMA7A peptides designed and synthesized according to the exemplary methods of Example 1 were used to generate multiple antibodies having affinity for SEMA7A. In some exemplary examples, a peptide represented by SEQ ID NO: 2 is used to generate polyclonal and monoclonal antibodies. In other exemplary examples, a peptide represented by SEQ ID NO: 3 is used to generate a peptide-protein conjugate to produce polyclonal and monoclonal antibodies against the 10 amino acid SEMA7A peptide. Because peptides alone can be too small to elicit a sufficient immune response, conjugation of the 10 amino acid SEMA7A peptide to a carrier protein can yield a more robust immune response. Therefore, any conjugate is contemplated herein to be used to bind to the peptides of interest and used to generate robust antibodies against SEMA7A. In certain examples, because some antibodies were raised against the peptide, the conjugation amino acid residues, and the carrier protein, choice of carrier protein as a conjugate during immunization differs from that to be used in the desired final assay. For example, KLH conjugates are used to immunize for antibodies when BSA will be used in the end-point assays. In some exemplary examples, a peptide represented by the sequence of SEQ ID NO: 3 is conjugated to the carrier protein (e.g., BSA, bovine serum albumin) but other suitable carrier proteins are also available and contemplated. In other exemplary examples, a peptide represented by the sequence of SEQ ID NO: 3 or SEQ ID NO. 2 is conjugated to the carrier protein KLH (keyhole limpet hemocyanin), for example.

In some exemplary methods, the 10 amino acid SEMA7A peptide and the 10 amino acid SEMA7A peptide-conjugated to a carrier protein were used to generate monoclonal antibodies. In brief, the peptides described herein were utilized to immunize mice which generated hybridomas from two different mice having serum that exhibited high level specificity for purified SEMA7A and for SEMA7A in human breast tumor cells and in human breast tissues. The hybridoma cultures generated from the immunized mice were screened, expanded and the selected clones were selected for production and purification of mouse monoclonal antibodies for SEMA7A.

In another exemplary method, the 10 amino acid SEMA7A peptide and the 10 amino acid SEMA7A peptide-conjugated to a carrier protein are used to generate polyclonal antibodies. In brief, polyclonal antibodies are produced by injecting one of the peptides herein into an animal (e.g., a rabbit). After being injected with the peptides to elicit a primary immune response, the animal is given a second, and even a third injection of the peptide to produce higher titers of antibodies against the 10 amino acid SEMA7A peptide.

Both polyclonal and monoclonal antibodies to the SEMA7A peptide were characterized. In brief, human SEMA7A protein is produced and purified from human breast cancer cells. Purified SEMA7A, alongside whole cell lysates from breast cancer cells with stable knockdown or overexpression of SEMA7A, provides samples for demonstrating binding specificity of the antibodies generated herein via Western blot and/or dot plot. Additionally, tissue lysates from wild-type and SEMA7A knockout mice are subjected to Western blot analysis using polyclonal and monoclonal antibodies to the SEMA7A peptide for validation of specificity. As further validation of specificity, polyclonal and monoclonal antibodies to the SEMA7A peptides were used to perform immunohistochemistry (IHC) on formalin-fixed human tissues collected from healthy breast tissue and cancerous biopsies.

### Example 3

In another exemplary method, antibodies detecting SEMA7A were used to diagnose breast cancer patients via a blood-based diagnostic method suitable for clinical use. Of note, a blood-based test for detecting SEMA7A for diagnostic purposes was not known before the discovery herein. In one example, the role of SEMA7A expression in breast cancer was investigated using a matched study to determine the relationship between human postpartum breast cancer (PPBC) and SEMA7A expression, and how SEMA7A was correlated with PPBC and clinically significant risk factors and outcomes. In brief, 113 cases from a Breast Cancer Cohort with an increased risk for metastatic recurrence in PPBC were assessed. Of those cases, 47 nulliparous patients and 66 PPBC patients were used in this exemplary study. The cases averaged 36 years of age with a range of 26-45. Overall, the groups were similar in age, race, stage, tumor size, grade, histology, biologic subtype, lymph node involvement (LN), lymphovascular invasion (LVI). All cases had invasive disease present and 39 (77%) contained adequate matched normal-adjacent breast tissue on the same tissue section. Based on available cases with tissue, the exemplary study examined nulliparous cases having 27 (57.5%) without and 20 (42.5%) with disease recurrence, distant and local-regional combined. For the PPBC cases, 42 (63.6%) with no recurrence, 19 with recurrence (28.8%), 4 other (6.06%) and 1 missing (1.5%).

In another exemplary method, using an antibody raised against amino acids 371-441 in the SEMA7A protein sequence, which recognized all isoforms of SEMA7A due to sequence homology, SEMA7A expression was compared with multiple breast cancer clinical characteristics that are known prognostic indicators across all 113 cases. In these examples, any antibody that binds to all or a fragment of amino acids 371-441 is contemplated to be of use. It was observed in this study that there were no differences seen in SEMA7A expression based on grade, stage, LN status, LVI, or biologic subtype when cases were analyzed as one combined group. Next, expression of SEMA7A was analyzed between the nulliparous and PPBC groups. First, it was observed that SEMA7A expression was significantly increased in the normal-adjacent tissues of the PPBC group compared with nulliparous and that this increase was most notable for those cases diagnosed within 3 years of last pregnancy and declined among cases that are further out from their last childbirth with significance lost by a diagnosis >3 to <5 years after birth (FIGS. 2A and 2B). Similarly, PPBCs had higher tumor SEMA7A expression than nulliparous; however, in the tumors, the level of SEMA7A expression remained higher even among cases with more time between last parturition and diagnosis, without a decrease as seen in the normal tissue, up to our 5-year current cut-off (FIGS. 2C and 2D).

### Example 4

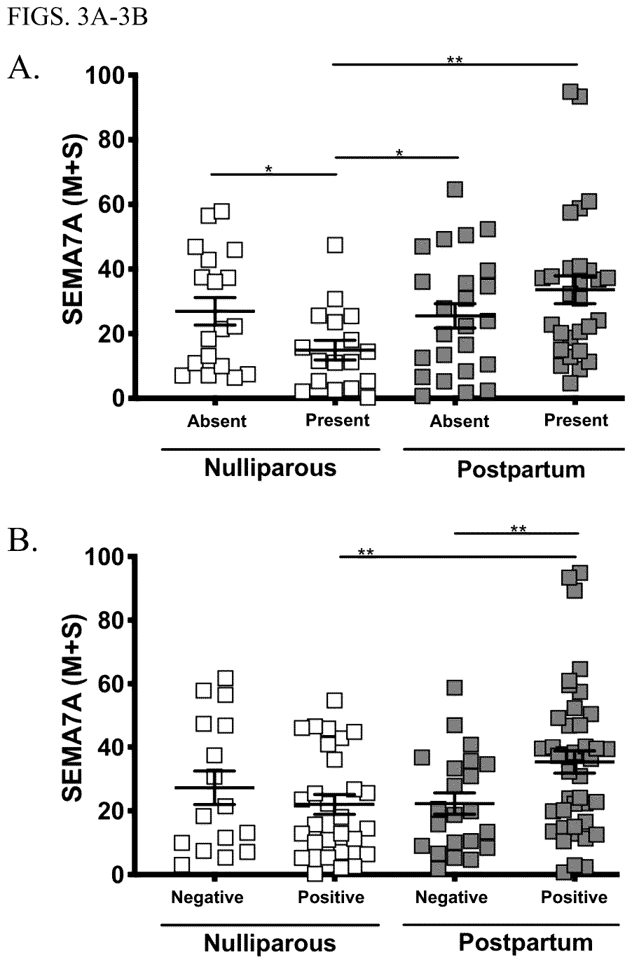

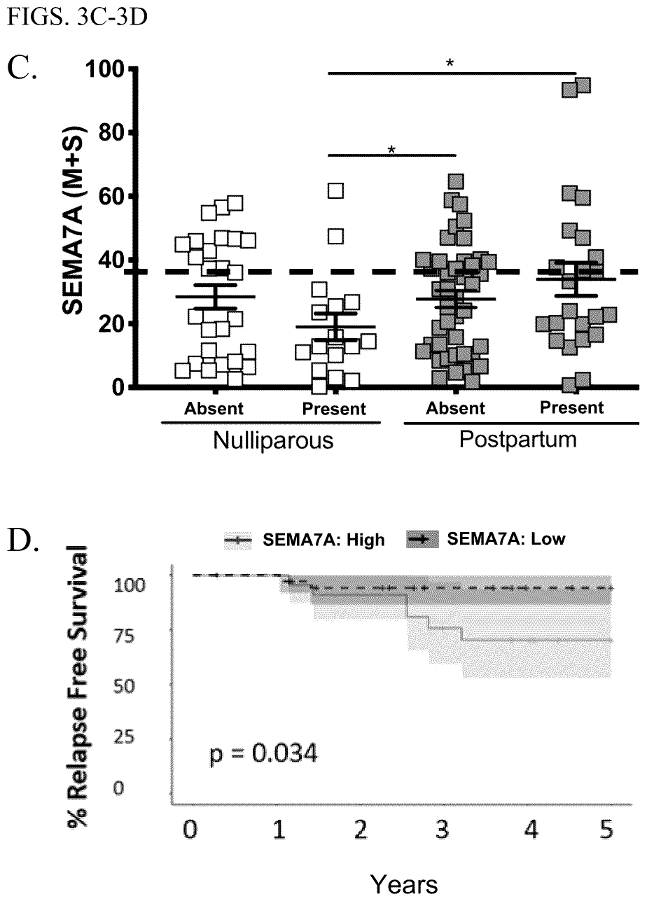

In another exemplary method, focusing on tumor analysis, SEMA7A expression did not differ between nulliparous and PPBC groups when compared for grade, stage, biologic subtype, hormone receptor status or recurrence in the entire cohort (FIG. 2E-2J). Additionally, when nulliparous patients who had experienced a pregnancy without childbirth were removed the difference remained and there was no difference based on the number of childbirths per patient (FIG. 2K-2L). When the cohort was separated by parity or equality status, SEMA7A did not differ based on stage grade or biologic subtype (FIG. 2M-2O) and when the nulliparous and PPBC were combined, there was no difference based on recurrence status (FIG. 2P). There was, however, a significant difference observed in the level of SEMA7A expression in PPBC cases that had evidence of lymphogenous spread compared to non-PPBC cases having lymphogenous spread. Specifically, postpartum patients with LN (lymph node) positivity exhibited elevated levels of SEMA7A compared to LN positive nulliparous patients. Similarly, SEMA7A expression was significantly higher in PPBC patients with evidence of LVI when compared to nulliparous patients (FIGS. 3A and 3B). SEMA7A expression was evaluated with respect to cases with and without breast cancer recurrence in the nulliparous and PPBC groups. It was observed that high SEMA7A expression correlated with poorer outcomes in PPBC, but not in nulliparous. Specifically, SEMA7A expression was observed to be highest in PPBC patients that had subsequently faced a breast cancer recurrence with local-regional or metastatic disease (FIG. 3C). SEMA7A was significantly lower in nulliparous patients with recurrence when compared to PPBC regardless of recurrence status. Thus, a Kaplan-Meier analysis was performed to determine a level of SEMA7A that was significantly associated with increased probability of recurrence in the PPBC group only. Less than 50% of the patients died during the follow-up period, thus, the median survival was not reached. It was determined that stratification of patients based on ˜36% SEMA7A positivity, defined as at least 36.1% positive pixels by quantitative IHC, generated recurrence free survival curves that are statistically significant different between the SEM7A high and low groups for PPBC patients (p=0.03) with 23 patients categorized into the SEMA7A high group and 36 patients in the SEMA7A low group (FIG. 3D).

Exemplary FIGS. 2A-2D illustrate SEMA7A expression was increased in normal and breast tumor tissues from PPBC. In brief, FIG. 2A depicts percent SEMA7A medium+strong (M+S) in normal adjacent breast tissues from women who were nulliparous (Nullip) or within 5 years of last childbirth (PPBC) at the time of tissue collection. FIG. 2B depicts data from FIG. 2A stratified by years since last childbirth. FIG. 2C depicts percent SEMA7A medium+strong (M+S) in breast tumor tissues. FIG. 2D depicts data from FIG. 2C separated by years since last childbirth. * p<0.05, one-tailed Student's t-test, mean+SEM are presented. Additional FIGS. 2E-2P demonstrate separation by additional clinical and patient parameters.

Exemplary FIGS. 3A-3D illustrate SEMA7A expression was a driver for poor outcomes in PPBC. In brief, SEMA7A (M+S) staining in patients stratified by (FIG. 3A) Lymphovascular invasion, (FIG. 3B) Lymph node involvement, and (FIG. 3C) Recurrence status. FIG. 3D depicts dichotomization of PPBC with an optimal cutoff of 36% M+S for SEMA7A high significantly predicts for decreased recurrence free survival.

These exemplary results provide support for SEMA7A as a biomarker for cancer (e.g., breast cancer). This data supports that SEMA7A drives prognosis, predicts treatment responsiveness, and is a targetable molecule for treatment. Further, SEMA7A is linked to poor prognosis for postpartum patients, ER+ patients, and ER— patients despite current treatment regimens. Therefore, identifying breast cancer patients positive for SEMA7A or elevated concentrations thereof can advance current treatment options for breast cancer patients by identifying patients not likely to respond to standard treatment avoiding unnecessary exposure to toxic anti-cancer agents while diverting treatments to more tailored treatments to the patients. In certain examples, these alternative treatments can include one or more of SEMA7A inhibition by treating a subject with one or more antibodies that bind SEMA7A and/or one or more anti-tumor agents such as Venetoclax or related agent thereof, or other B-cell lymphoma-2 (BCL-2) inhibitor thereof or P13K pathway inhibitors (e.g., LY294002, Buparlisib, or the like). The B-cell lymphoma 2 (BCL-2) family is divided into three groups based on their primary function (1) anti-apoptotic proteins (BCL-2, BCL-XI, BCL-W. MCL-1, BFL-1/A1), (2) pro-apoptotic pore-formers (BAX, BAK, BOK) and (3) pro-apoptotic BH3-only proteins (BAD, BID, BIK, BIM, BMF, HRK, NOXA, PUMA, etc.). Any BCL-2 inhibitor is contemplated of use herein.

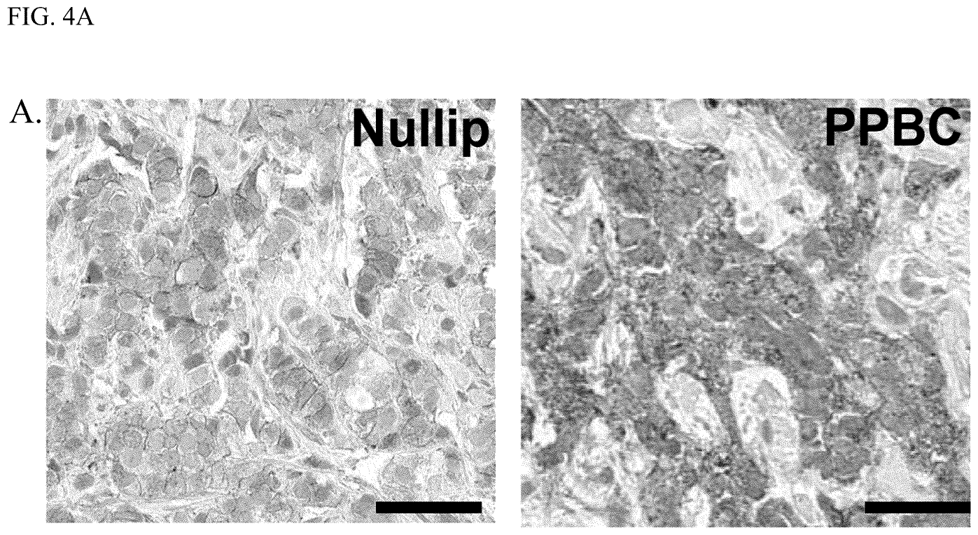

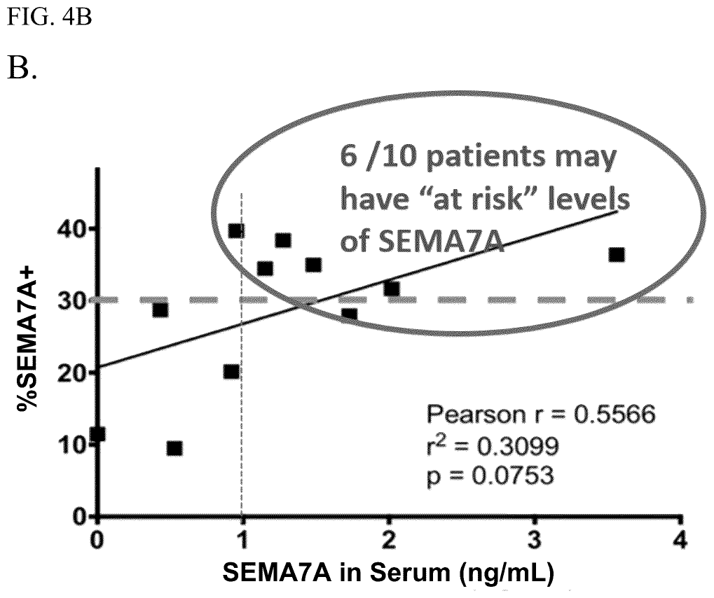

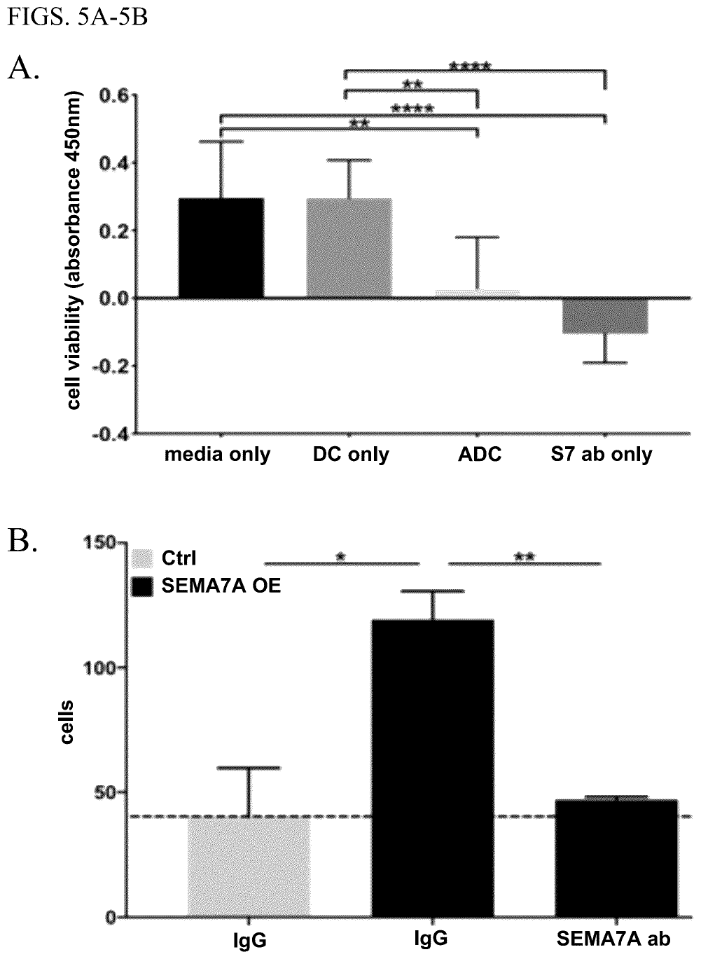

In another exemplary method, a threshold for SEMA7A levels in blood as a biomarker for breast cancer prognosis in humans has been established. Mammary tissue biopsies and matched blood samples from age-matched, cancer-free, nulliparous, and postpartum women were collected. Mammary tissue biopsies and matched blood samples are analyzed with IHC and ELISA, respectively, to determine baseline SEMA7A levels in normal breast tissue and blood. Blood was collected from healthy donors to help establish base blood SEMA7A levels in healthy individuals compared to breast cancer patients. To determine the relationship between SEMA7A presence and/or levels and breast cancer prognosis, matched blood samples were compared to tumors from both PPBC and nulliparous patients where the patients contributing to the samples in the study also provided complete reproductive history, clinicopathologic annotation, updated clinical outcomes for local and distant metastasis, and treatment information. Using SEMA7A antibodies capable of binding any SEMA7A, over 120 mammary tissue and tumor samples were stained and quantified for SEMA7A. After identifying patients with known levels of SEMA7A expression in their tissues, matched serum was tested with SEMA7A ELISA to correlate SEMA7A levels to those in the tumor. Both tumor and blood SEMA7A levels were compared to patient history to evaluate whether SEMA7A expression levels are predictive of prognosis. SEMA7A antibodies used in ELISA assays detected SEMA7A in the blood. Data demonstrated that detecting as low as 1 ng/mL of SEMA7A in the blood via ELISA was associated with having >30 percent expression in the tumor tissue, which in this example, was critical for predicting recurrence in breast cancer prognosis. In certain cases, patients having a history of breast cancer or currently having breast cancer identified as further having SEMA7A or elevated levels of SEMA7A in their samples can indicate the need for immediate therapeutic intervention and/or alterative intervention compared to standard cancer therapies (FIG. 4).

These exemplary results demonstrating antibodies detecting SEMA7A in the blood provide a diagnostic tool for assessing whether this high-risk population, patients with SEMA7A+ breast cancer, should be given alternative, more aggressive or combination therapies to improve outcomes and survival of patients annually.

### Example 5

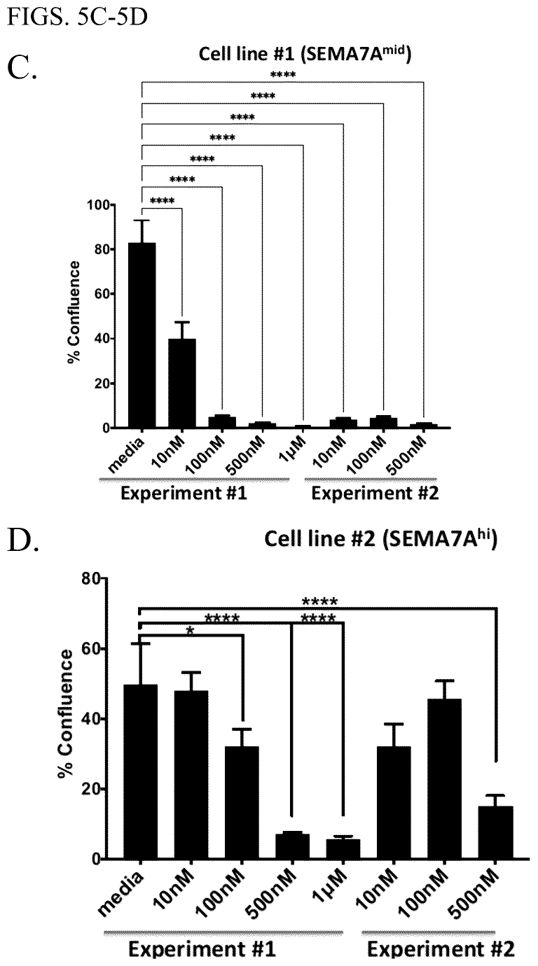

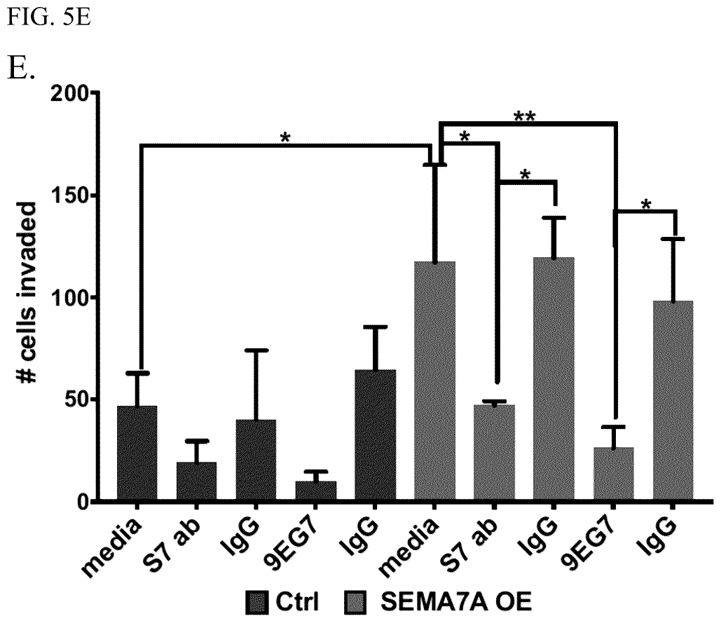

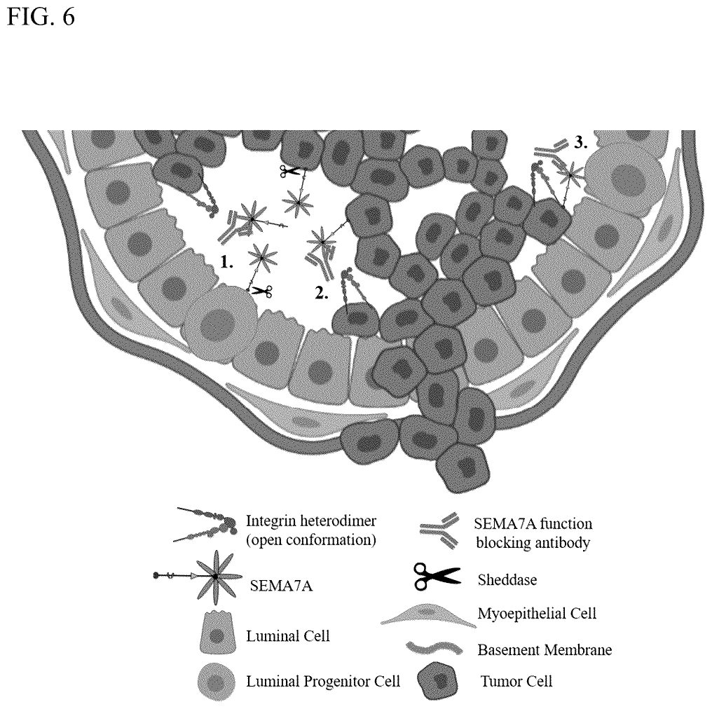

In another exemplary method, antibodies against SEMA7A were tested against tumor cell lines in a subject. Human and mouse mammary carcinoma cell lines were used to determine if SEMA7A monoclonal antibodies against amino acids 371 to 441 of SEMA7A disclosed herein (“SEMA7A mAb”) that binds to SEMA7A to reduce cell proliferation, viability, invasion, and migration in vitro. In brief, human derived MD Anderson metastatic breast cancer 231 and Michigan Cancer Foundation 10A ductal carcinoma in situ (MDA-MB-231 and MCF10ADCIS, (respectively) cell lines were used to determine if the SEMA7A mAb reduced cell viability in dose- and time-dependent manners. The MDA-MB-231 and MCF10A lines are models of triple negative BC. SEMA7A mAb-treated (e.g., SC-374432 C-6; Santa Cruz Biotech; immunizing peptide QPIPTETFQVADRHPEVAQRVEPMGPLKTPLFHSKYHYQKV SEQ ID NO:4) cells had reduced cell viability in vitro (FIG. 5A). Additionally, SEMA7A mAb was conjugated to the toxin saporin. The saporin-conjugated SEMA7A mAb was internalized into the cells following treatment and cell viability was also reduced in the cells (FIG. 5A). Finally, the SEMA7A mAb reduced cellular invasion in vitro (FIG. 5B). A novel SEMA7A monoclonal antibody, SmAbH1 (immunizing peptide ADRHPEVAQR SEQ ID NO:2), decreased breast tumor cell confluence in MDA231 (cell line #1) and MCF10ADCIS (cell line #2) in a dose dependent manner and cellular invasion in vitro as well as an antibody blocking beta-1 integrin, a receptor for SEMA7A, that was previously proven to block tumor cell invasion (FIG. 5C-5E). A model depicting mechanism of action demonstrates how a SEMA7A mAb has therapeutic potential (FIG. 6).

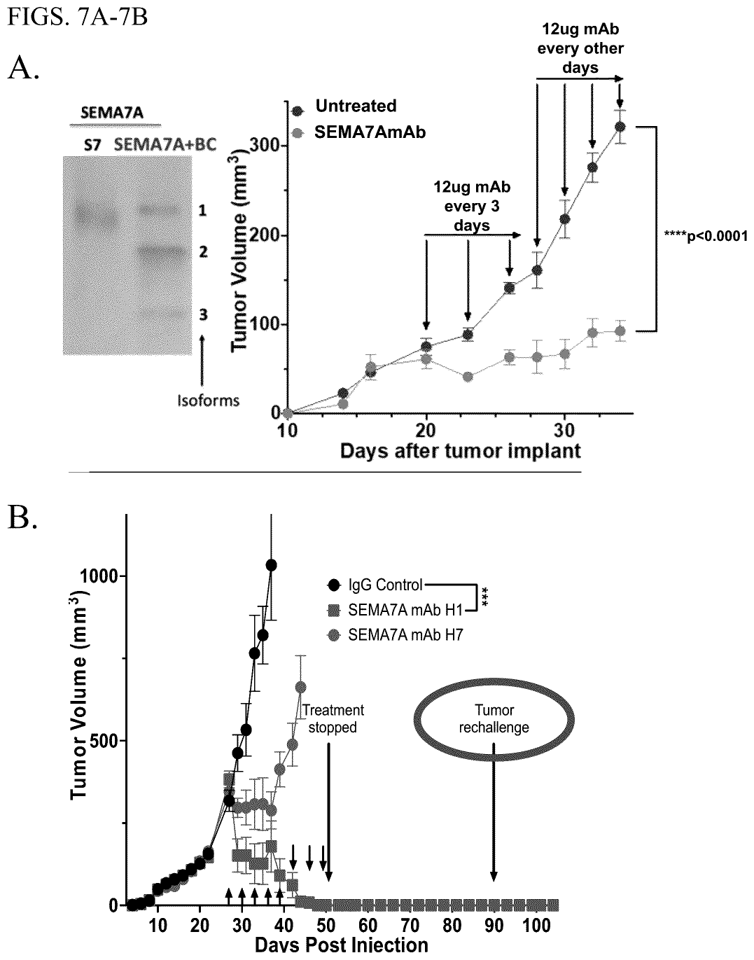

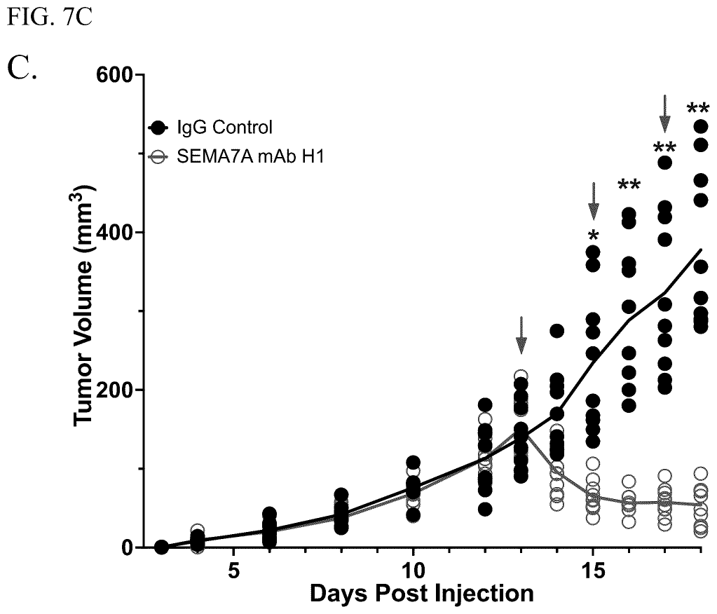

For initial in vivo studies with SmABH1, SEMA7A OE E0771 mouse mammary carcinoma cells were injected into mice. Twenty days after carcinoma cells were injected, SmAbH1 (e.g., 12 ug) was administered intraperitoneally to the mice every three days until day 36 post-carcinoma cell injection. Tissues were collected at day 36. Tumors, lungs, and LNs were harvested and processed for IHC and flow cytometry to determine the effect of SEMA7A mAb therapy on cells of the tumor microenvironment (TME). In an additional experiment, tumors were treated at day 28 post-injection with two different novel SEMA7A directed antibodies, monoclonal antibody clones H1 and H7. Fully regressed tumors were removed from treatment at day 50 and the animals were re-challenged with an additional tumor inoculation at day 90 to assess SEMA7A mAb effects. Finally, to examine changes in immune cells in the tumor microenvironment, tumors were treated at day 13 post-inoculation and harvested at day 18. During this time period, tumors were actively regressing (FIG. 7A-7C).

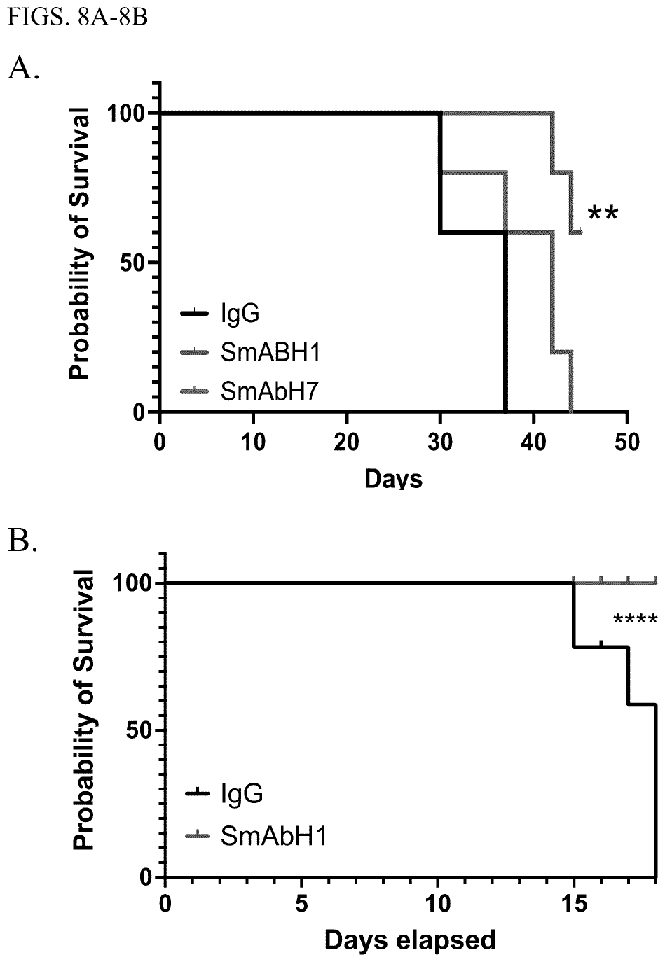

In these examples, in vivo studies indicated that treatment of SEMA7A OE tumors with SEMA7A mAbs markedly inhibited tumor growth (FIG. 7A-7B). Additionally, mice treated with SEMA7A mAbs had a significant increase in survival time. Treated mice had double the survival time from about 35 days to about 75 days compared to untreated mice (FIGS. 8A-8B). This observation equates to an increase in probability of survival for humans of about 2.4 years to about 5.2 years using SEMA7A mAbs treatment alone.

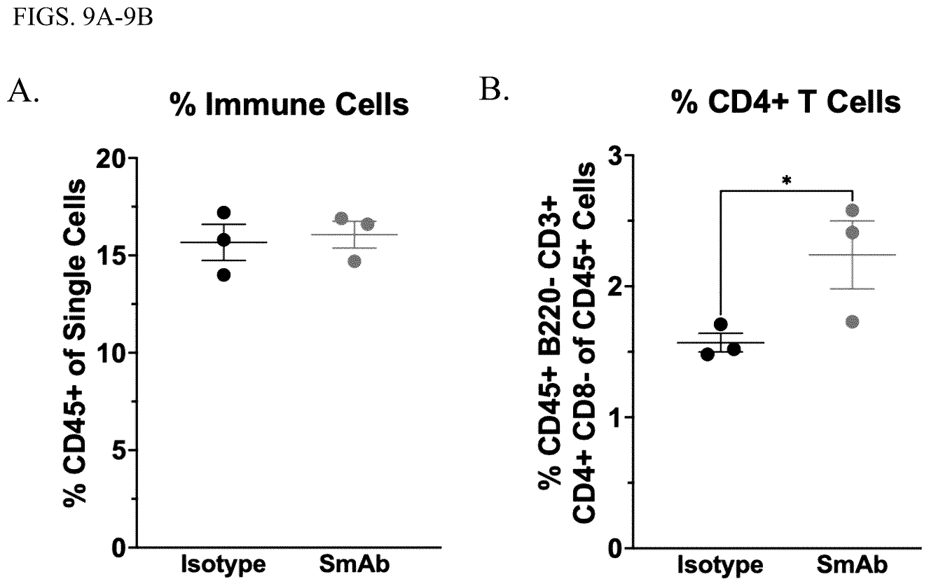

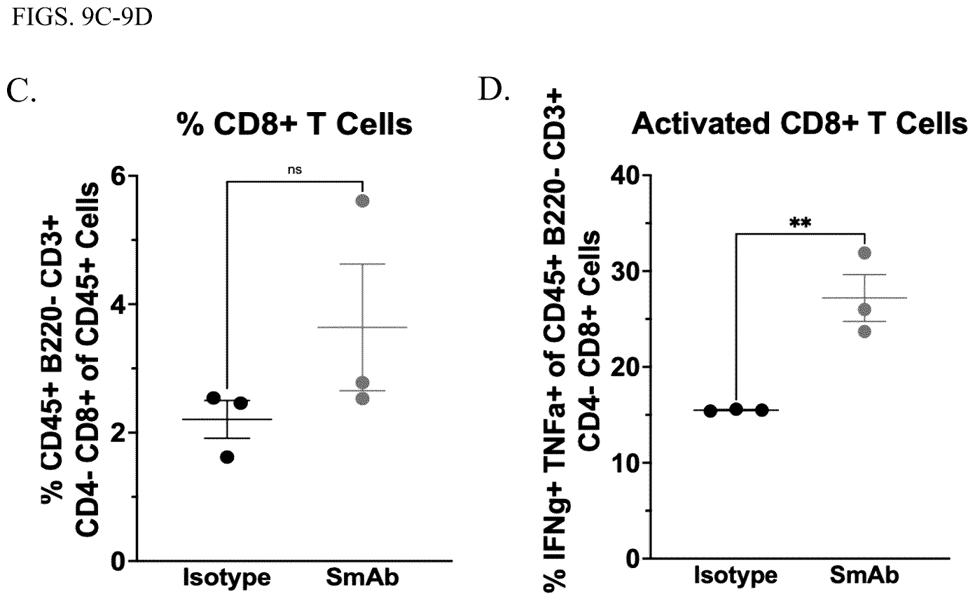

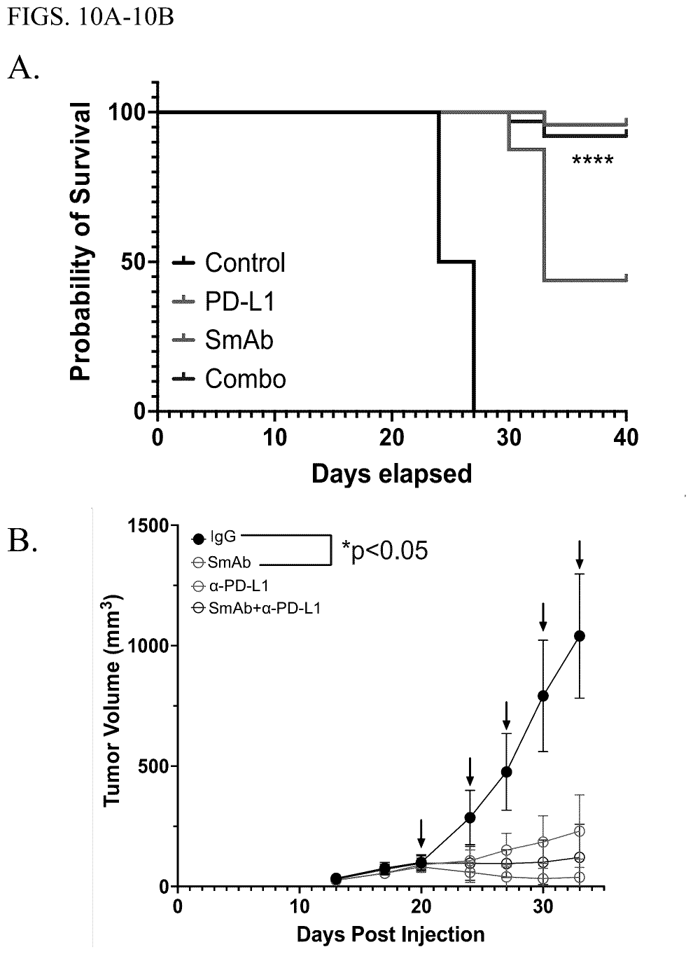

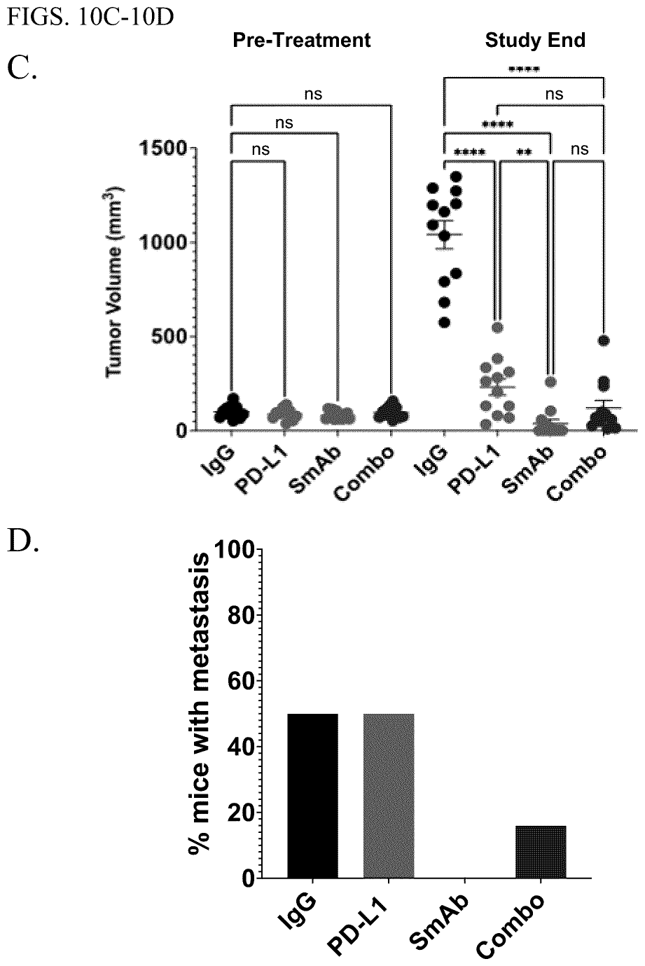

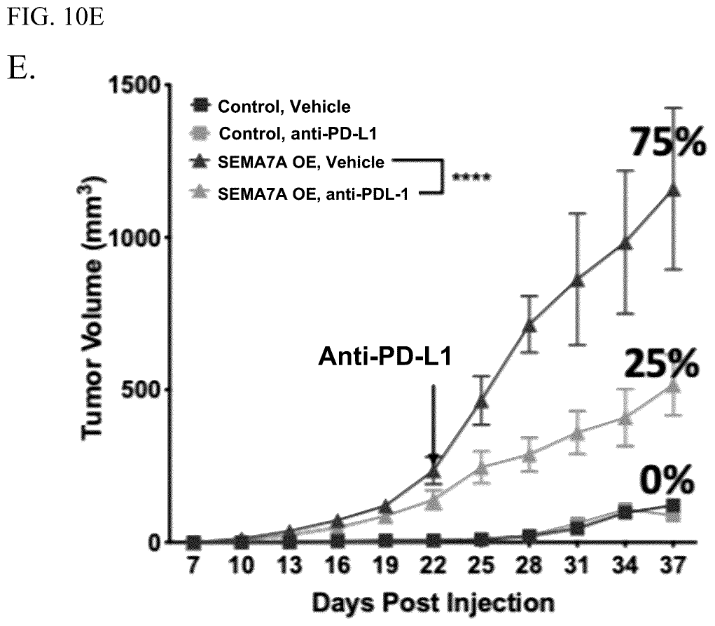

Flow cytometry analysis from in vivo studies indicated that treatment with SmAbH1 (SEMA7A monoclonal antibody) resulted in an increased presence of activated T cells in the tumors (FIGS. 9A-9D). Further, SmAbH1 was tested alone or in combination with a known immunotherapy (anti-PD-L1). These experiments demonstrated an increase in activated T cells in tumors exposed to SmAbH1 alone which was superior at reducing both tumor growth and metastasis when compared to anti-PD-L1 and the combination of both antibodies (FIGS. 10A-10E). Additionally, SEMA7A expressing tumors when exposed to anti-PD-L1 treatment alone did not fully regress, but metastasis was reduced (FIG. 10E).

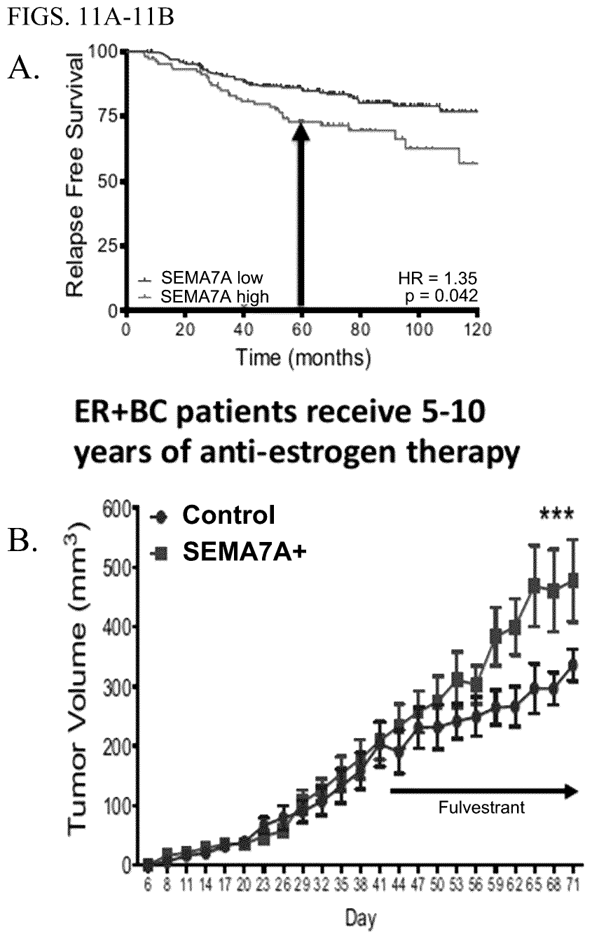

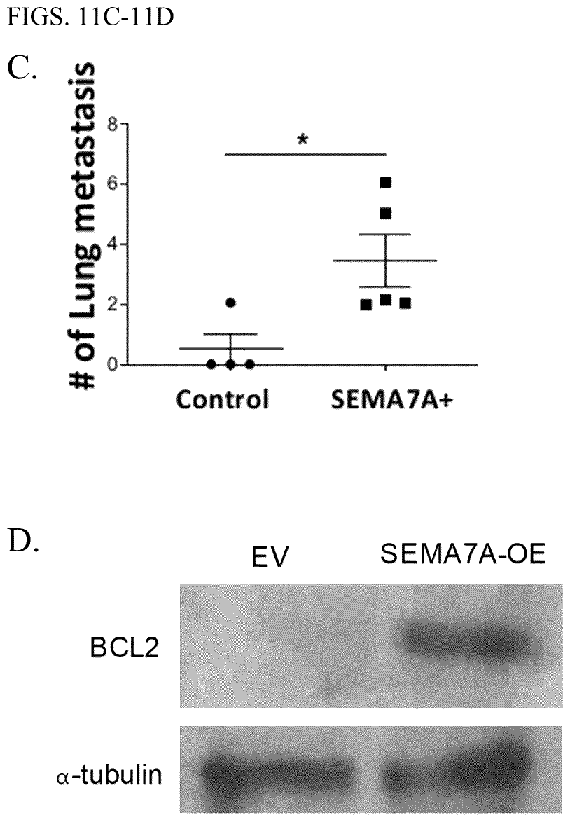

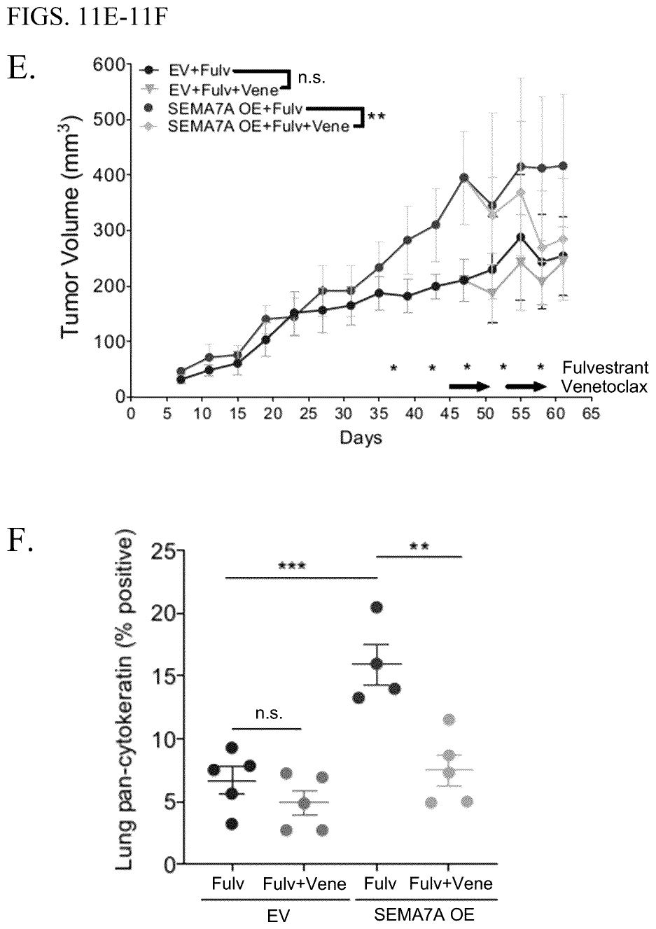

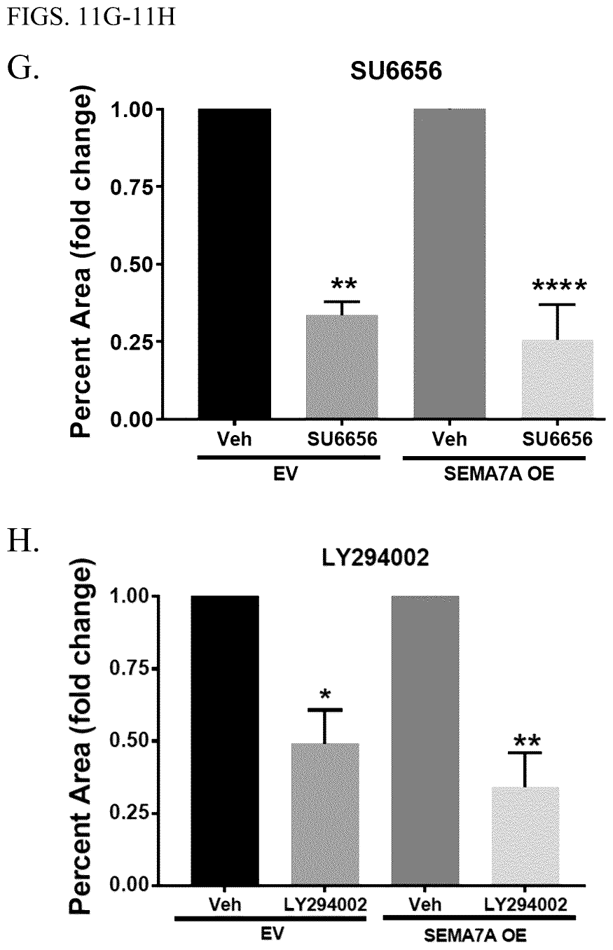

In another exemplary method, ER+SEMA7A expressing tumors are resistant to anti-estrogen-based therapy (Fulvestrant), anti-CDK4/6 therapy (Palbociclib), and chemotherapy but are sensitive to inhibition of cell survival pathways. In certain methods, Venetoclax (a BCL-2 inhibitor) was assessed in SEMA7A-expressing tumors for targeting anti-apoptotic molecule BCL-2, which also reduces metastasis in the human MCF7 model of ER+ breast cancer (FIG. 11A-11H). In one example (11G) Clonogenic growth of MCF7 EV or SEMA7A OE cells treated with a Src inhibitor (SU6656) and (11H) a PI3K inhibitor (LY294002) are disclosed as potential combination treatments with the monoclonal antibodies disclosed herein to SEQ ID NO:2 and/or 3 or conjugate thereof.

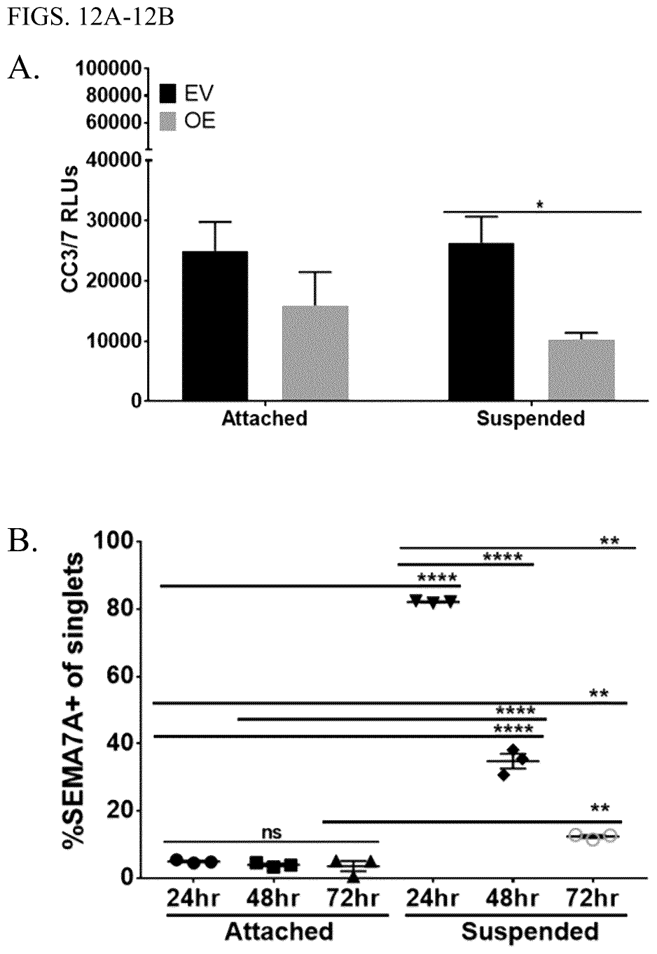

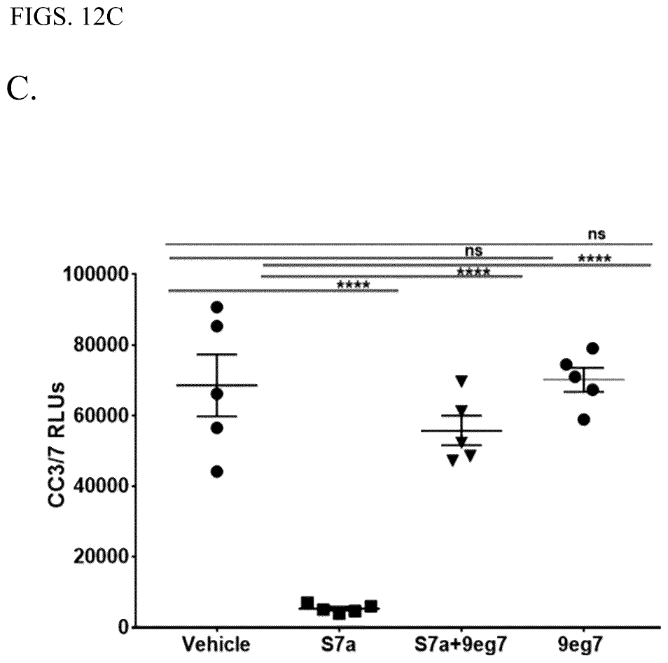

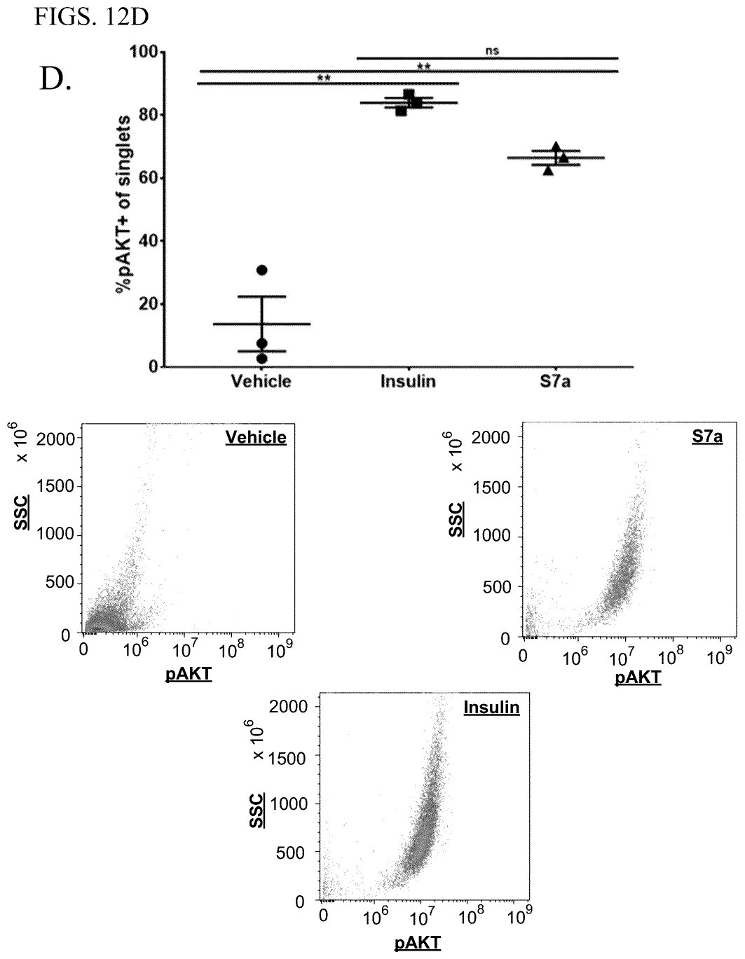

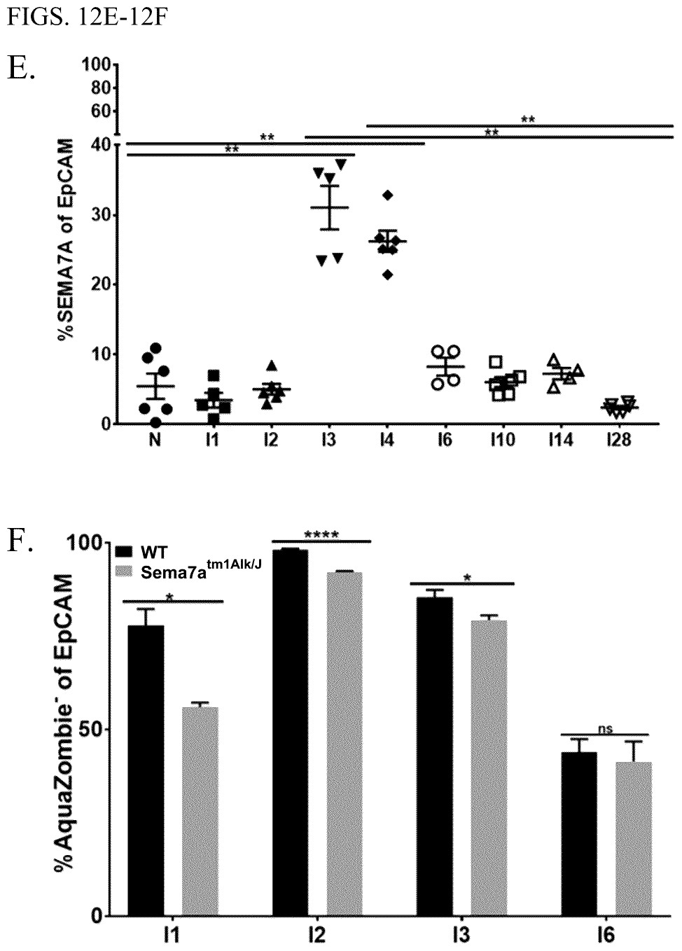

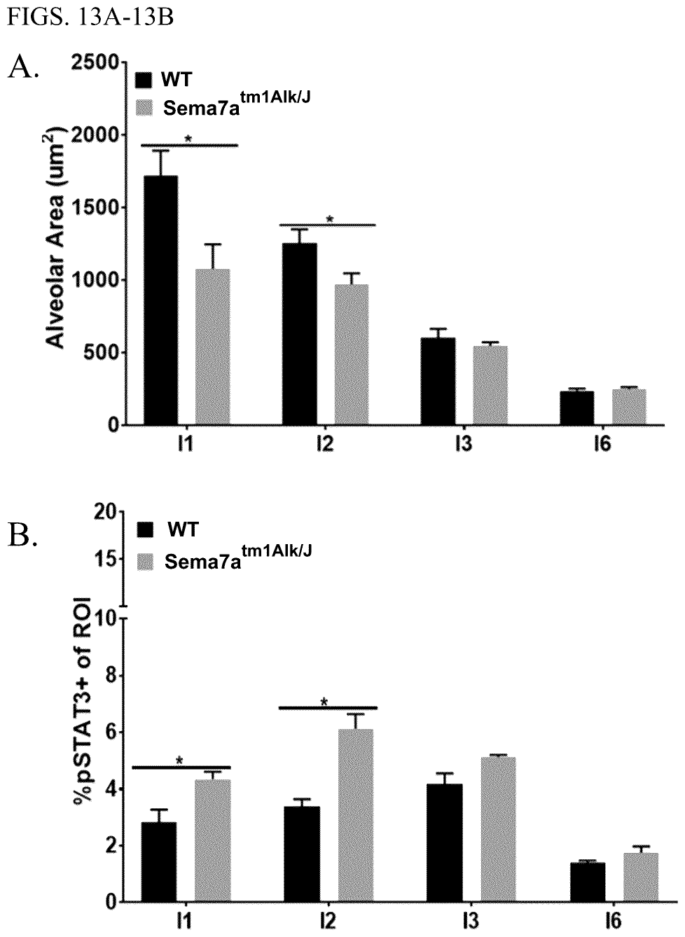

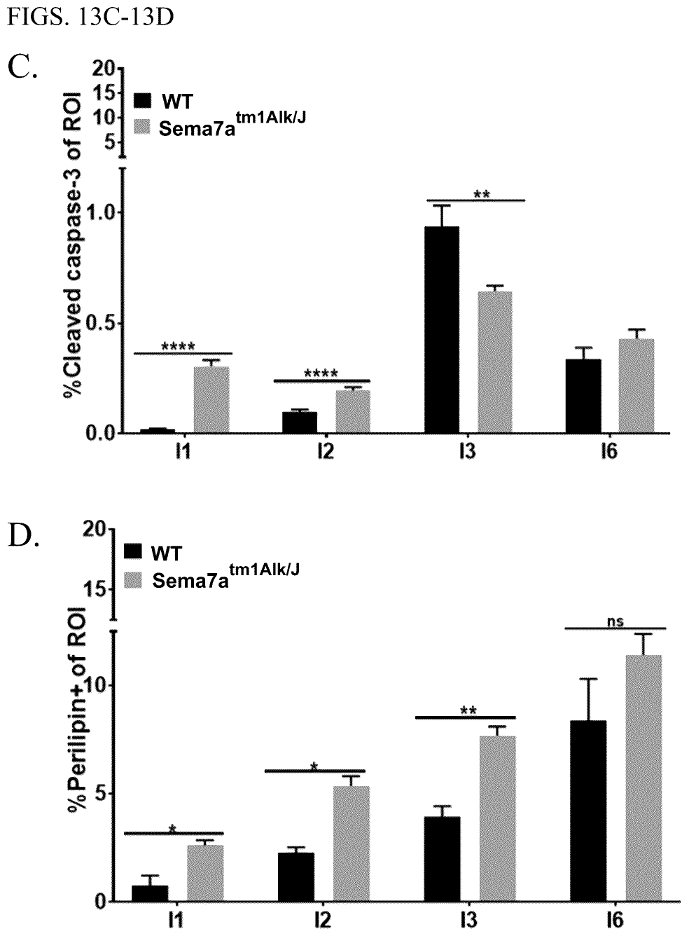

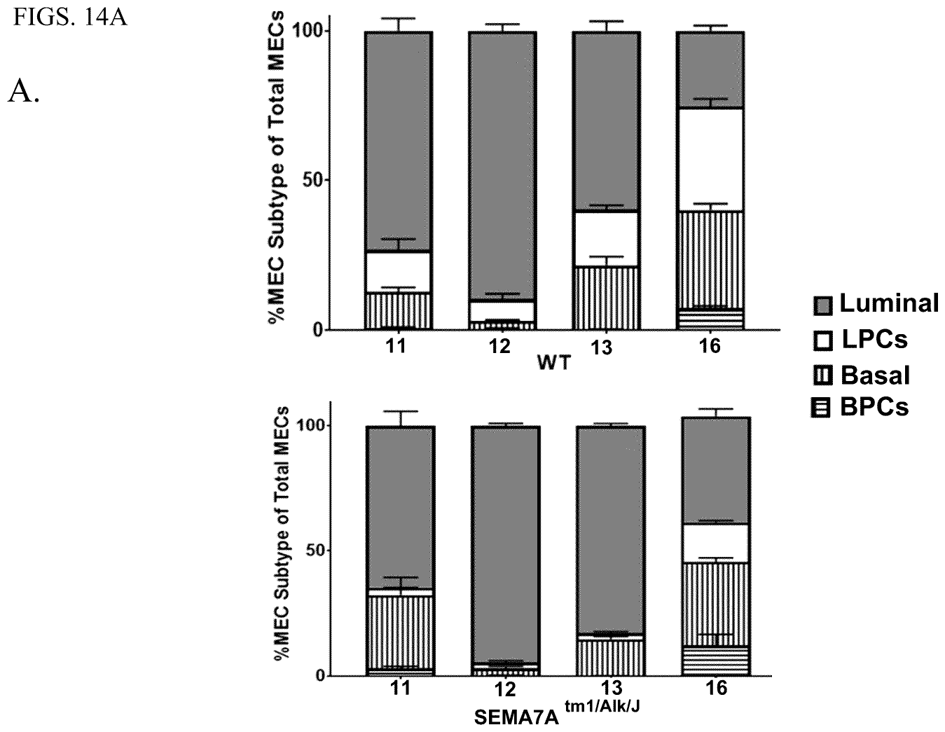

In another exemplary method, the role of SEMA7A in maintenance of mammary progenitor and stem cell populations was demonstrated by analysis of SEMA7A during postpartum mammary gland involution, the process by which the lactating mammary gland remodels to the pre-pregnancy state. A process that has been demonstrated as a key driver of PPBC. During this process the milk secreting cells undergo apoptosis induced by anoikis, or death by detachment from the extracellular matrix, but the progenitor cells must survive for subsequent rounds of lactation. SEMA7A was demonstrated to be key for anoikis resistance, which was mediated in part by activation of beta-1 integrin and Akt in cultured mammary epithelial cells and SEMA7A was highest at involution day 3. Furthermore, cell survival was reduced in SEMA7A knockout mice (Sema7a^{tm1Alk/J}) when compared to wild-type (WT) (FIGS. 12A-12F). Moreover, postpartum involution was accelerated in SEMA7A knockout mice further supporting its role in cell survival in the postpartum mammary gland (FIG. 13).

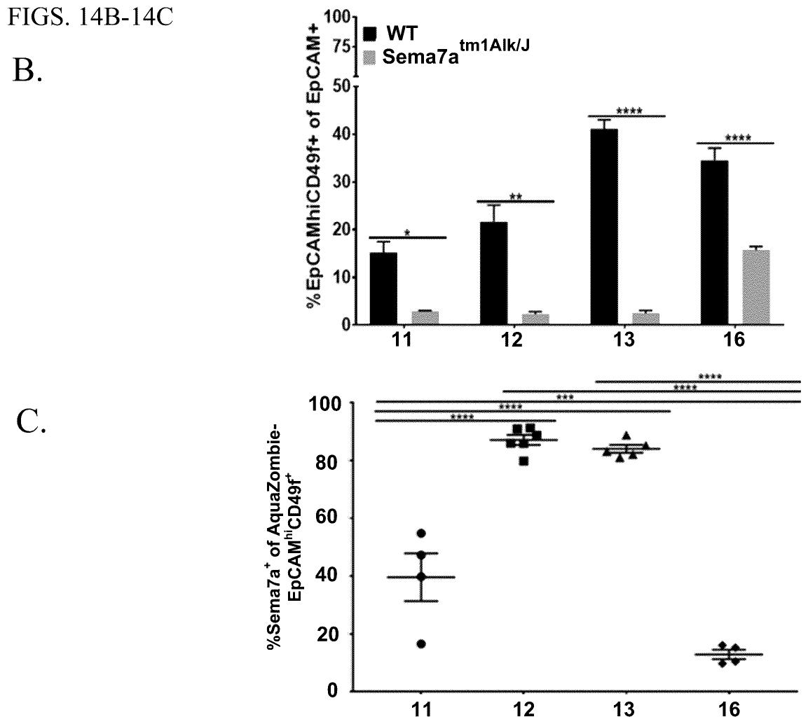

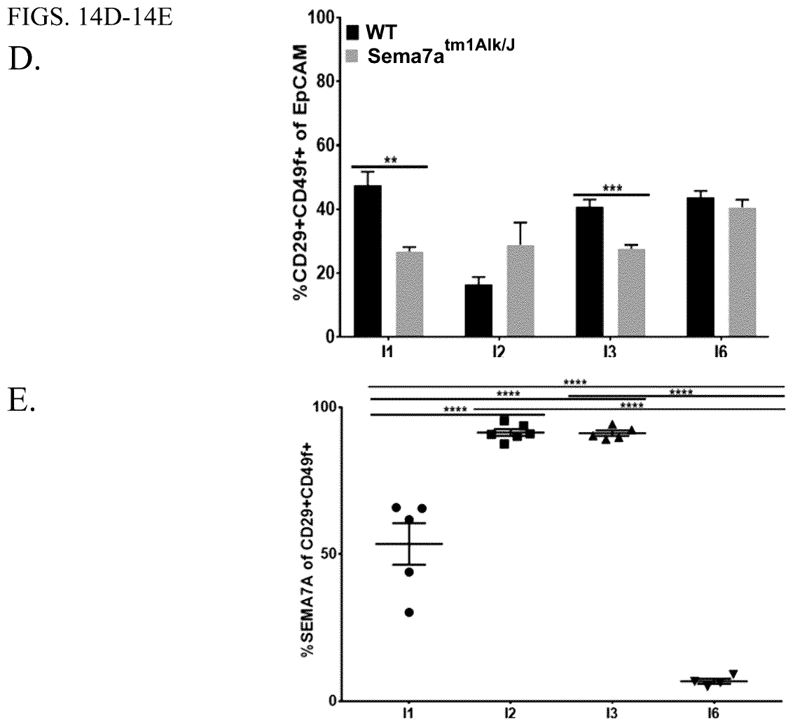

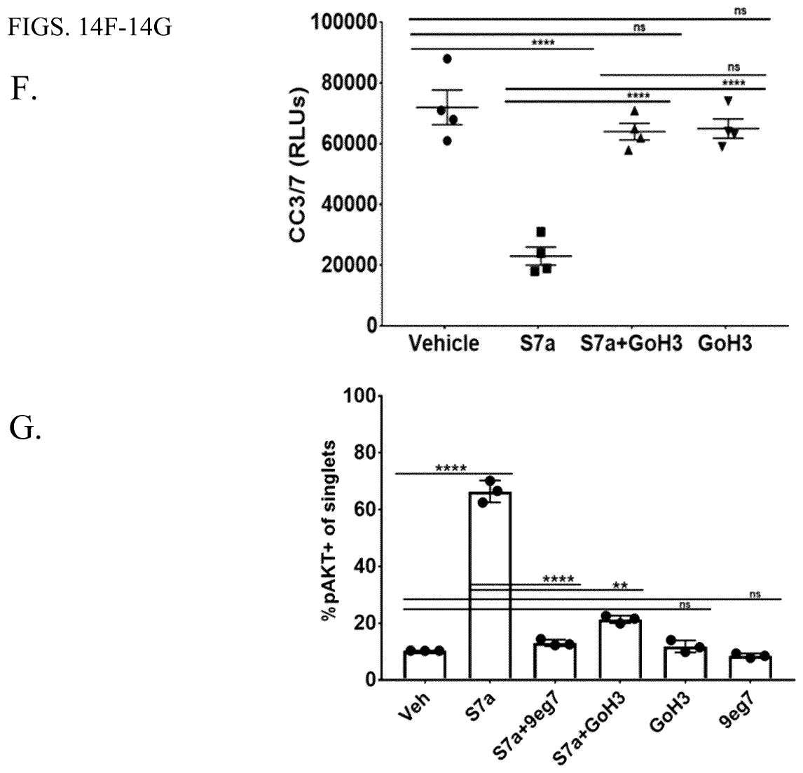

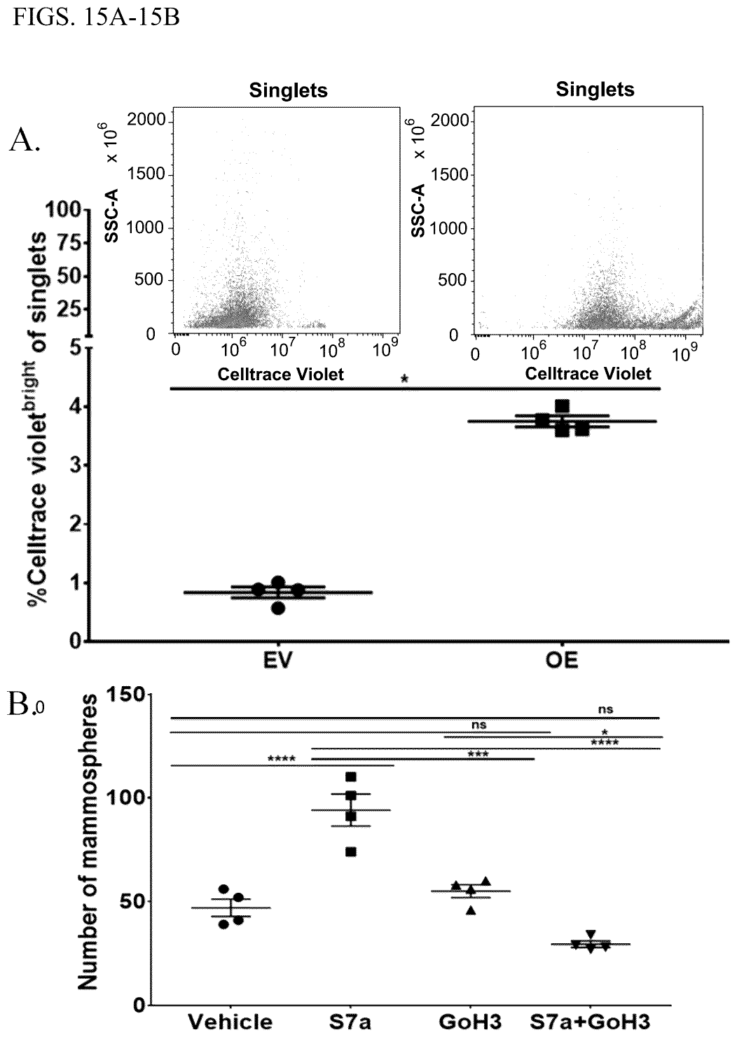

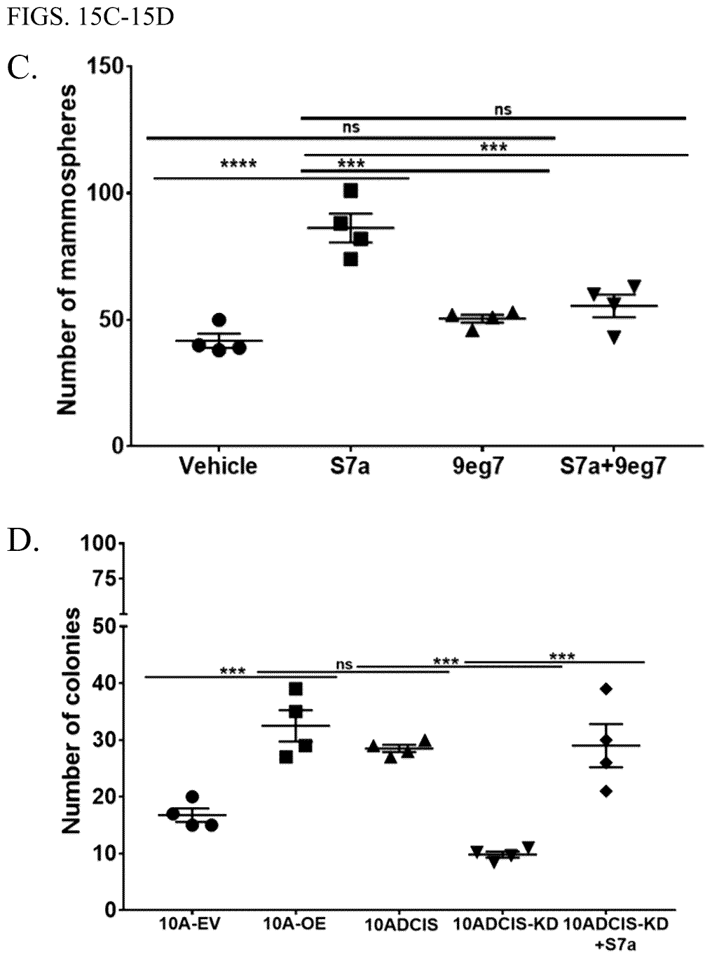

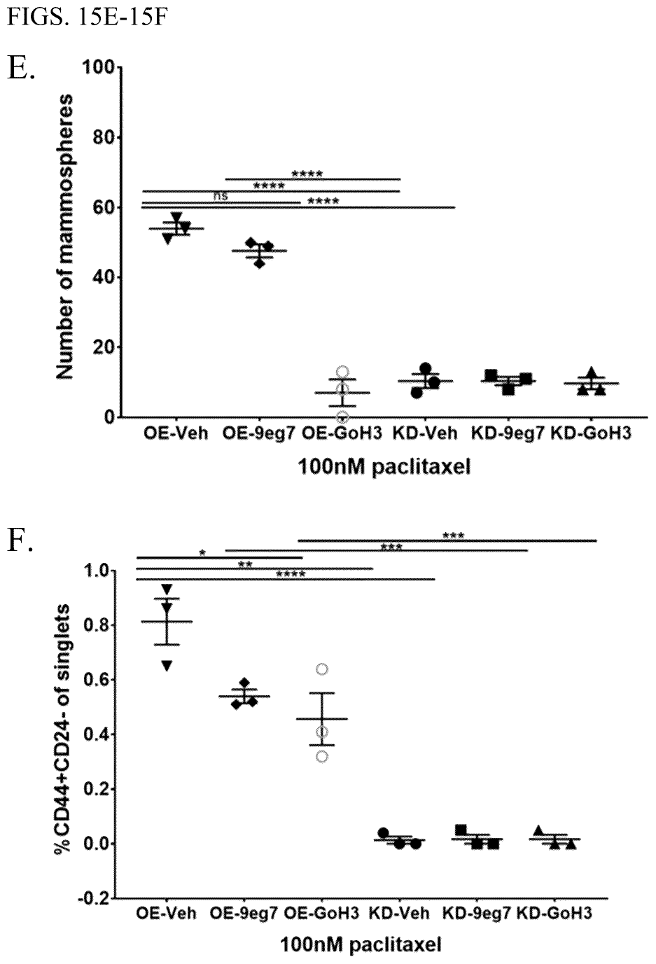

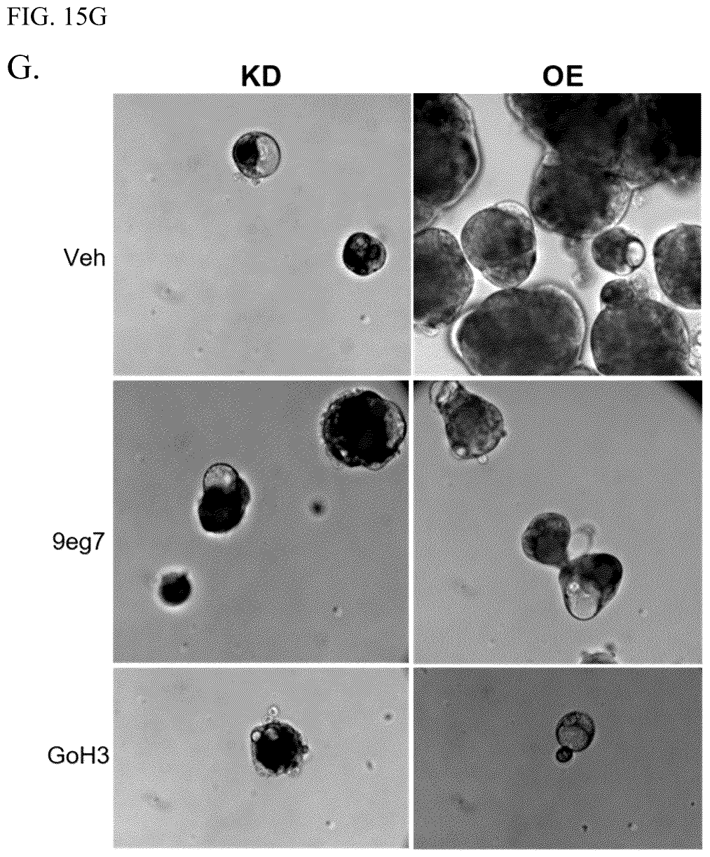

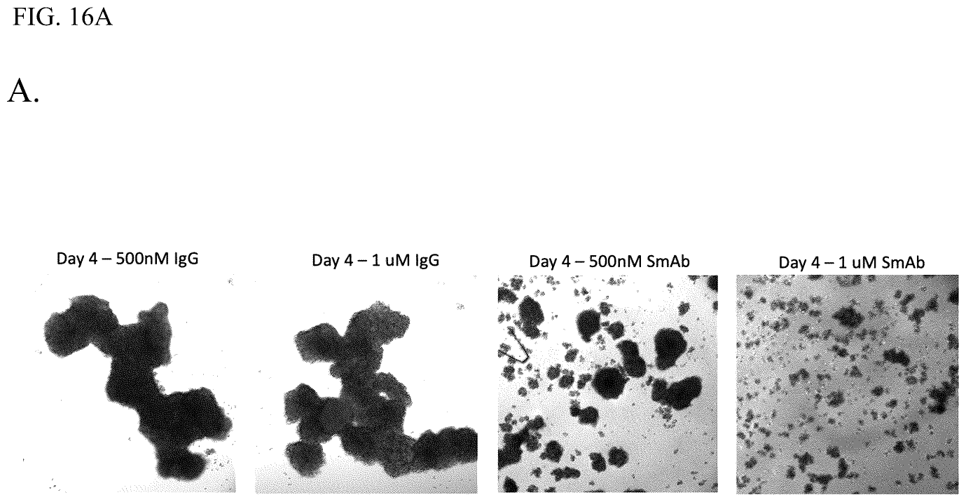

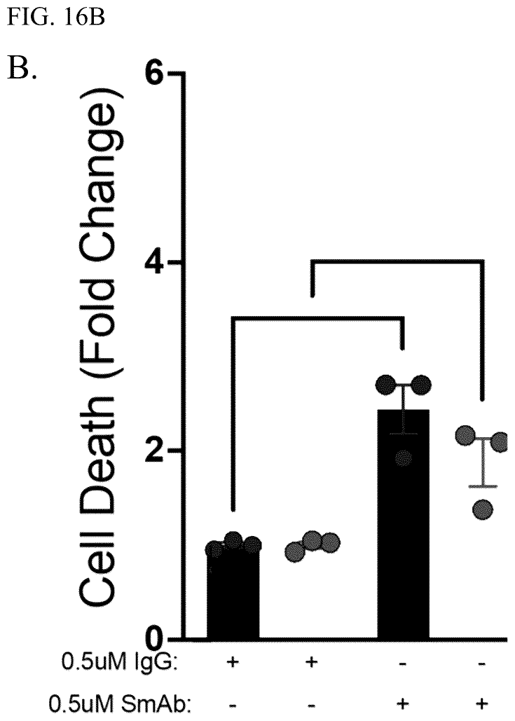

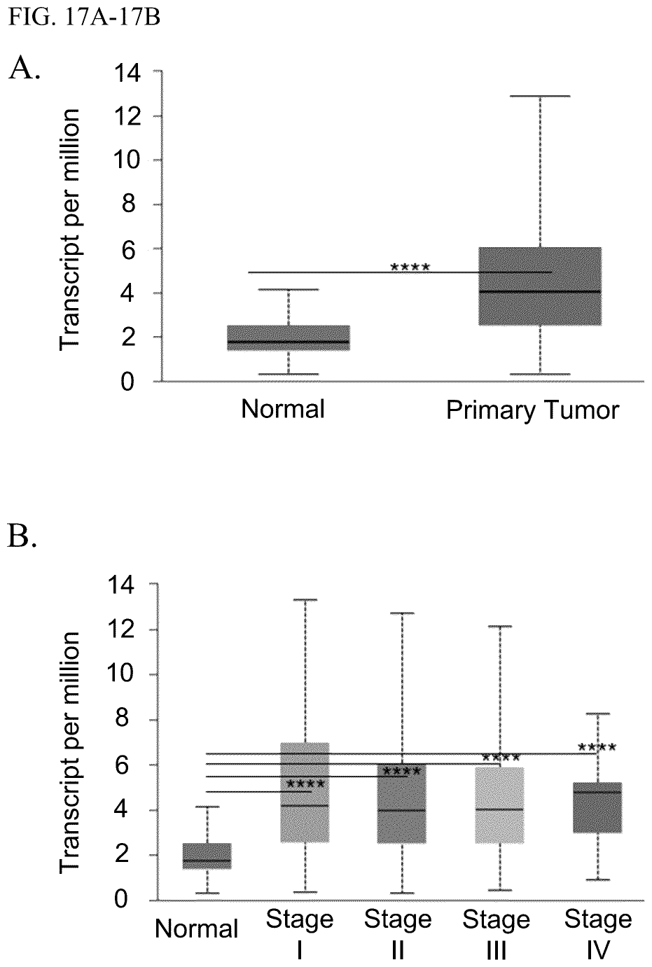

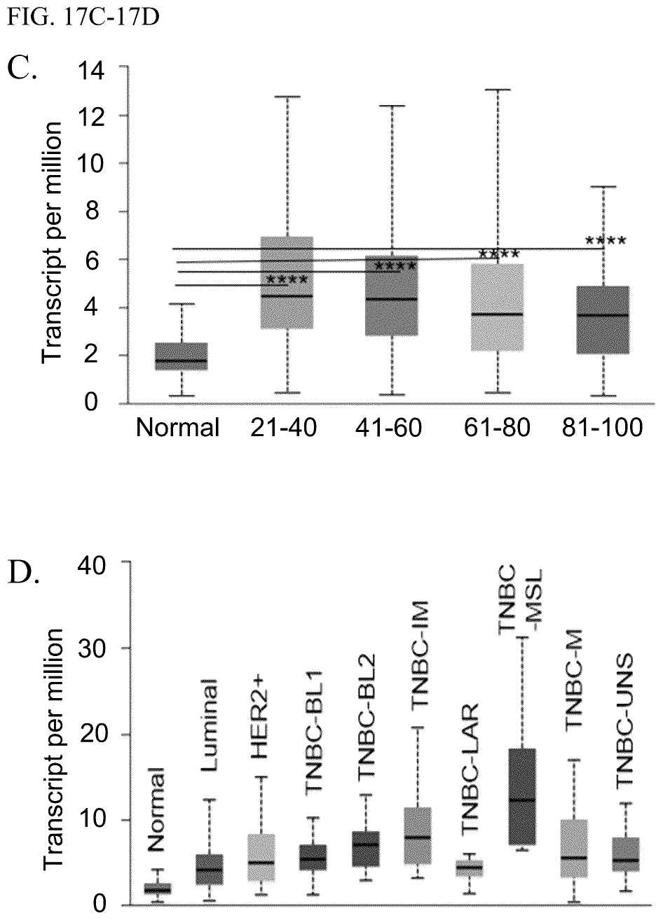

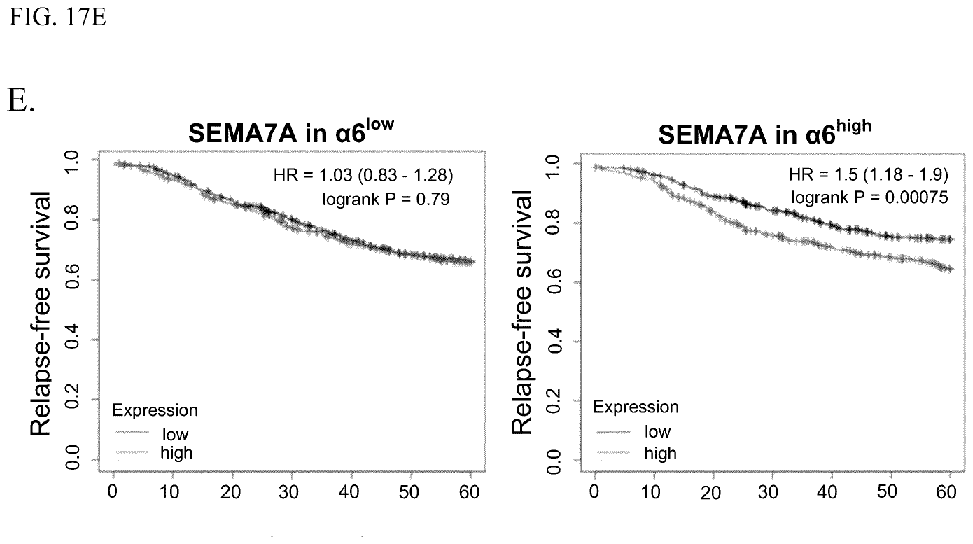

In another exemplary method, it was demonstrated that SEMA7A is specifically expressed on mammary progenitor/stem cells and required for their survival (FIGS. 14A-14G). Additionally, it was observed that SEMA7A was sufficient to promote stem cell phenotypes, including mammosphere formation, anchorage independent growth and resistance to chemotherapy, which is a property of cancer stem cells. Finally, SEMA7A expressing cells display increased functions and markers of cancer stem cells (FIG. 15). However, treatment of cells with SmAbH1 was sufficient to reduce mammosphere formation and induce cell death (FIGS. 16A-16B).

These exemplary methods suggest that patients with SEMA7A expressing tumors can be resistant to current standard of care therapies and that alternative therapies, such as anti-PD-L1/PD-1 and use of an exemplary BCL-2 inhibitor: Venetoclax—or other therapies that target cell survival pathways, should be used alone or in combination with anti-SEMA7A antibodies or monoclonal antibodies to SEMA7A or epitopes targeted herein. Consistent with this, analysis of the cancer genome atlas (TCGA) reveals that patient tumors express high levels of SEMA7A, regardless of stage, and levels of SEMATA are highest in younger patients and in patients with immune and stem cell enriched tumors. Finally, co-expression of SEMA7A and alpha-6 integrin, a partner for beta-1 integrin and stem cell marker, have decreased relapse free survival; this data supports a role for SEMA7A in promoting relapse in breast cancer patients regardless of current therapies.

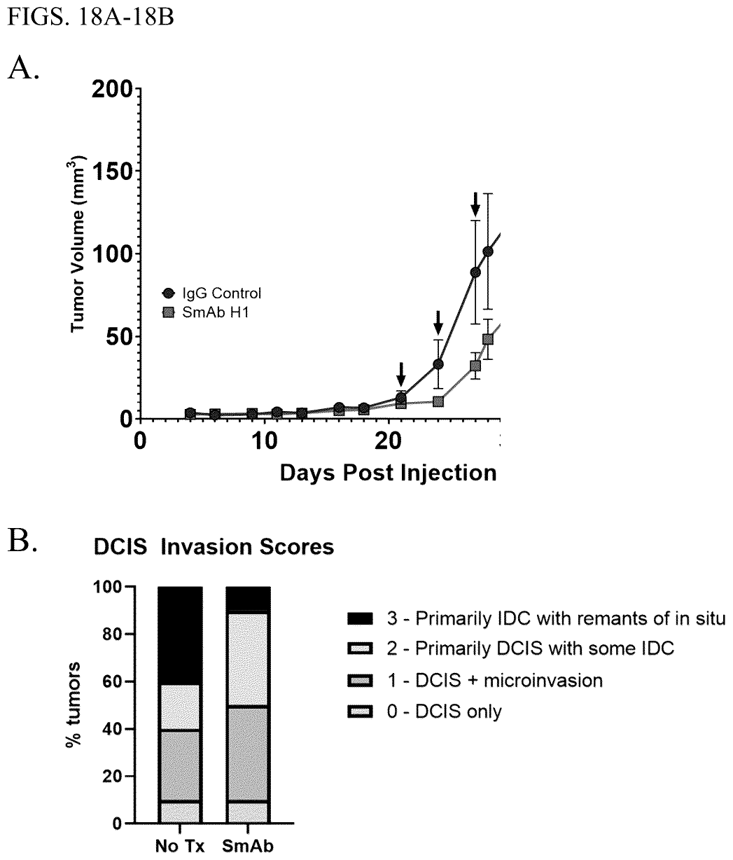

In another exemplary method, the ability of the SEMA7A monoclonal antibody (SmAbH1) to block or inhibit tumor growth and invasion of ductal carcinoma in situ (DCIS) lesions was demonstrated in a mouse model, which suggests that SEMA7A could be targeted at the earliest stages of breast cancer in order to reduce or prevent cancer progression, metastasis, and promote tumor cell killing (FIGS. 18A-18B).

### Example 5

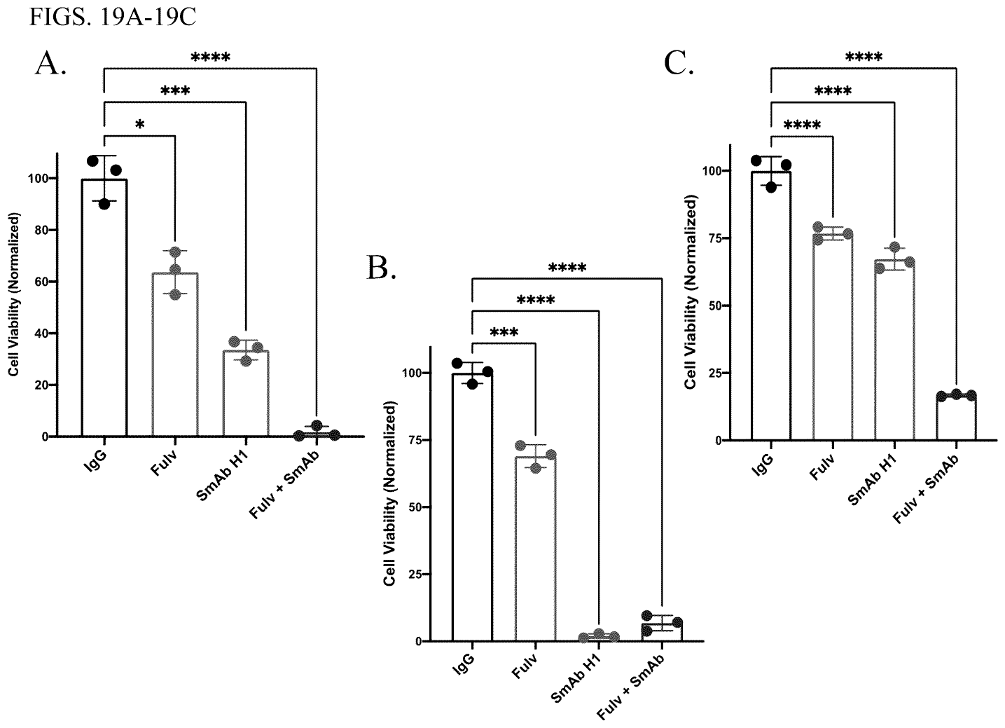

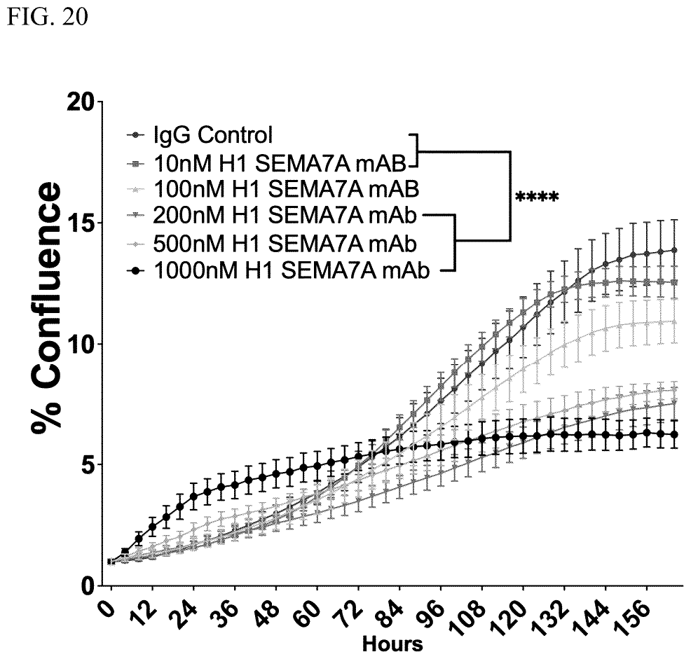

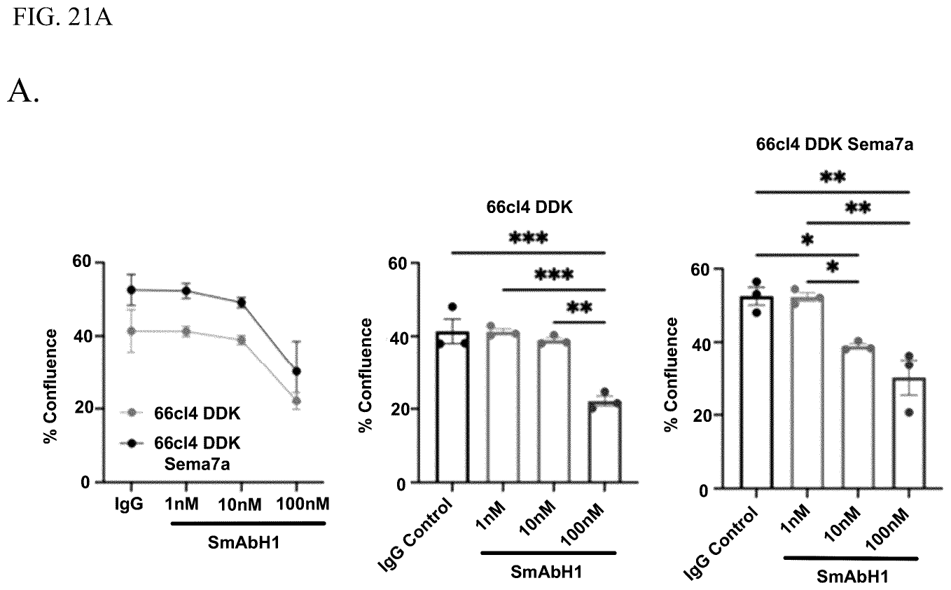

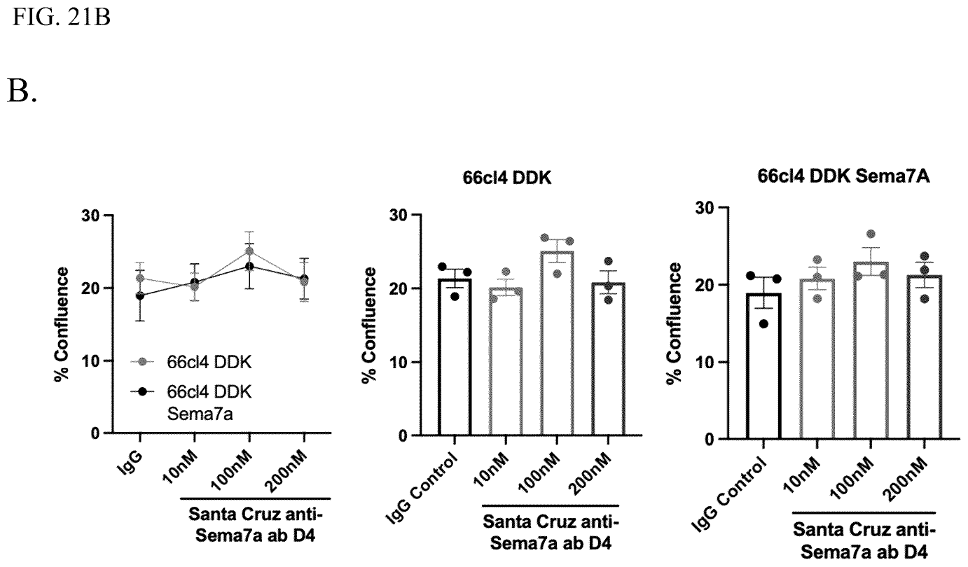

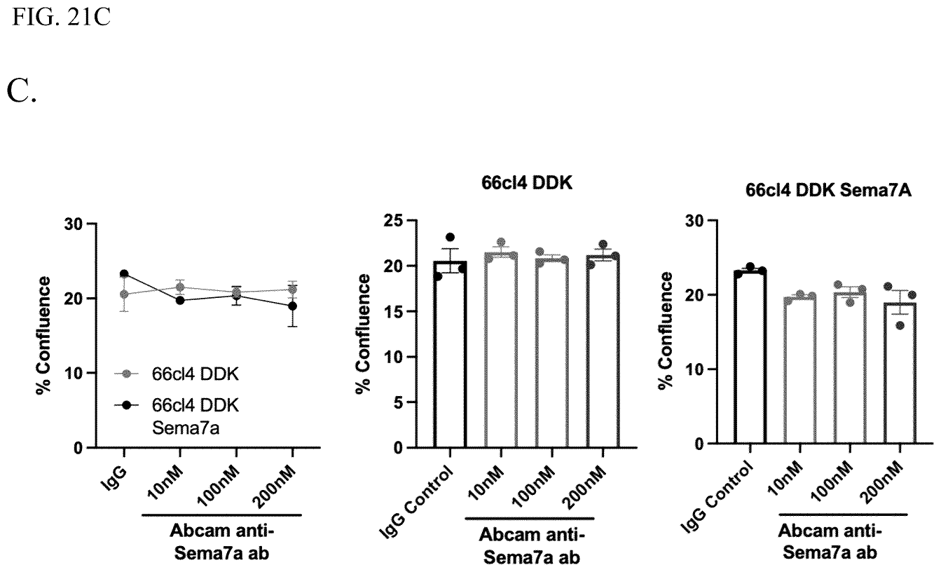

In another exemplary model, direct tumor cell killing by SEMA7A monoclonal antibodies (SmAbH1) was demonstrated in vitro. ER+SEMA7A expressing tumor cells (MCF7, TC11 and SSM2) viability was more significantly reduced by SEMA7A monoclonal antibody (SmAbH1) compared to anti-estrogen therapy (fulvestrant) (FIGS. 19A-19C). Additionally, in a model of ER-SEMA7A expressing tumors decreased viability was observed with SEMA7A monoclonal antibody (SmAbH1) in a concentration and time dependent manner, modeled in this example (FIG. 20). In another method, in the same model, the ability of the SEMA7A monoclonal antibody (SmAbH1) to decrease cellular confluence, a measure of cell viability, was observed which was greater than commercially available SEMA7A directed antibodies, (e.g., SC-374432; Santa Cruz Biotech and Ab23578; Abcam) (FIGS. 21A-21C). This data supports that the monoclonal antibodies to the epitope claimed herein can cure SEMA7A-related conditions and work better than the commercially available antibodies.

These exemplary methods suggest that SEMA7A monoclonal antibody (SmAbH1) could be utilized for treating both ER+ and ER− breast cancers alone or in combination with currently approved therapies.

In one exemplary method, data mining for combined molecular score in multiple types of cancer reveals that SEMA7A plays an important role for other types of cancer besides breast cancer. It was found that other cancers can benefit from reducing SEMA7A expression or translation such as endometrial, skin, liver, brain, ovarian, stomach, mesothelioma but the highest correlation was leukemia and pancreatic other than breast cancer (data not shown). It is noted that leukemia can be treated with the BCL-2 inhibitor (e.g., venetoclax) in addition to a SEMA7A monoclonal antibody against SEQ ID NO:2 or 3 or conjugate thereof. Given this observation, leukemia could also be treated with an anti-SEMA7A antibody disclosed herein alone or in combination with a BCL-2 inhibitor.

### Materials and Methods

### Immunohistochemistry

Tissues were formalin fixed and paraffin embedded as previously described. Four μm sections of paraffin-embedded human tissue were deparaffinized and pretreated with 1×Dako Target Retrieval solution under pressure for 5 minutes. Slides were prepared in a Dako Autostainer using semaphorin 7a primary antibody (SEMA7A C-6, Santa Cruz). Immunoreactivity was detected using Envision+ Mouse secondary antibody (Dako). 3,3′-diaminobenzidine was used as the chromogen (Dako, 10 min). Hematoxylin was used as the counterstain (Dako, 6 min). The majority (>80%) of cases had Ki-67% index staining centrally performed as previously described.

Staining quantification was done using Aperio ImageScope software (Leica Biosystems). Histological sections were digitally scanned using Aperio ScanScope3 equipment. Each section was assessed for normal-adjacent tissue and invasive breast cancer (IBC), and peritumoral lymphatic vessel density in a blinded manner. Tissues were subsequently annotated for representative regions of each tissue category present. Annotated regions were analyzed for percent positive staining using a proprietary color deconvolution algorithm created in ImageScope. Percent positive was calculated as the sum of total medium and strong (M+S) positive signal, divided by the total annotation area, and multiplied by 100.

### Statistical Analysis

Two-sample independent t-tests were used to compare the distributions of the continuous outcomes between Nullip vs PPBC groups, and the paired comparisons between subgroups; Fisher's exact tests were conducted to compare the distributions of the categorical variables between Nullip vs PPBC groups, and the paired comparisons between subgroups. For all tables and dot plot graphs, continuous outcomes are expressed as the mean+/−SEM, and categorical outcomes are presented as frequencies and the percentages. For Kaplan Meier analysis, Recurrence Free Survival (RFS) is defined as the time from the date of diagnosis to the date of local recurrence, reginal recurrence, or the last date of follow-up, whichever comes first. The non-parametric log rank test was conducted to compare the recurrence free survival curves of SEMATA low vs high groups for PPBC patients and for Nulliparous patients separately. The optimal cutoff level of SEMA7A were obtained using R package ‘survminer’ 20. The cutoff level of SEMATA is set to 36.1, which is slightly above the average observed in the PPBC group and can describe the bimodal feature of the distribution of the SEMA7A in this group reasonably well and is also biologically and clinically plausible. P-values less than 0.05 were deemed statistically significant. The datasets generated during and/or analyzed during the current study are available from the corresponding author on reasonable request.

### SEMA7A ELISA

Human SEMA7A ELISA (Biomatik, Cat #EKU07269) was used according to manufacturer's instructions. Breast cancer patient serum samples were obtained from our collaboration with Dr. Virginia Borges and collected under an IRB-approved protocol. Samples were selected based on tumor level expression of SEMA7A and sample availability.

### Generating SEMA7A-Antibody Drug Conjugate

Fab-ZAP Mouse antibody drug conjugation kit (KIT-48) was obtained from Advanced Targeting Systems (San Diego, CA), used with human anti-mouse-SEMA7A antibody (sc-374432, clone C6, lot #K1512 SantaCruz Biotechnologies, Dallas TX) according to the manufacturer's instructions, with modifications discussed in results section.

### Cell Culture and Reagents

Overexpression plasmid (SEMA7A-Fc) was generously gifted from R. Medzhitov (Yale University, New Haven, CT). Control plasmid (pcDNA3.1) was obtained from H. Ford (CU Anschutz Medical Campus, Denver, CO). All other overexpression plasmids (p304-V5-Blasticidin and V5-SEMA7A) were obtained from the Functional Genomics Core at the CU Anschutz Medical Campus and overexpression was confirmed via qPCR and Western blot analysis.

### Monoclonal Antibody Generation

Mice (5) were injected with KLH-conjugated Sema7a Peptitde. Serum was then collected for testing. ELISA, Western Blot and IHC using serum to detect purified SEMA7A and peptide. Mice were then boosted with peptide and testing serum until detection is at a desired level. Spleens were then harvested and B cells from spleens were fused with myeloma cells to create hybridomas. Positive hybridomas were then selected and monoclonal antibody can be harvested from the supernatant.

### Proliferation Assays

### In Vitro Growth, Proliferation Rate, Adhesion, and Motility Assays

For proliferation assays, cells were plated at 2000 cells per well of a 96-well dish. Four pictures of different regions in each well were taken every four hours for 48 h using an IncuCyte ZOOM (Essen BioScience, Ann Arbor, MI, USA). Percent confluence was determined using the IncuCyte ZOOM software (Essen BioScience). 66cl4 Sema7A DDK cells were plated in a 96-well plate in triplicate at 1,500 cells/well. They sat down over 24 hours and then were treated with 1000 nM IgG control, 10 nM SmAbH1, 100 nM SmAbH1, 200 nM SmAbH1, 500 nM SmAbH1, or 1000 nM SmAbH1. The plate was promptly transferred to the IncuCyte where photos were taken and analyzed every 4 hours for 7 days. For additional treatment studies in MCF7, TC11 and SSM2 cells (10,00/well) were plated in a 24-well plate in complete media and allowed to sit down over 24 hours. At 24 hours after plating, cells were treated with 500 nM IgG control, 500 nM SmAbH1, 8 nM Fulvestrant or the combination. The plate was incubated at 37 C for 48 hours, then fixed with 10% NBF and stained with crystal violet.

### Trans-Well Invasion Assays

For transwell invasion assays, 25,000 cells per well were plated in triplicate precoated transwell inserts with 1% horse serum as chemoattractant and quantified by Image J.

### In Vivo Assays

E0771 model: Female C57/BL6 mice were injected orthotopically into the left and right #4 mammary fat pads with 200,000 E0771 SEMA7A OE mouse mammary carcinoma cells. Tumors were measured every day once tumors became palpable (3 days post injection). When the average tumor volume for the full cohort (all 10 mice) reached 150 mm³, mice began treatment with either SmAb H1 or IgG2 isotype control. Treatment was administered every other day by intraperitoneal injection at a concentration of 12 ug, 50 ug or 250 ug (F50 μg in 100 μl total volume (final concentration=2 μg/ul). For PD-L1 studies PD-L1 was co-delivered at 250 μg.

DCIS model: Female SHO mice, aged 6-8 weeks old, are injected with 250,000 MCF10ADCIS GFP human breast cancer cells into both #4 mammary fat pads, then palpated and measured three times a week with digital calipers and tumor visualization is aided by use of the illumatool light source with GFP filter so we can accurately measure tumors. Treatment with SmAb started at day 21 post injection. SmAb treatment is given by IP injection every third day. Weights are taken every treatment day and mice are monitored for weight loss and other health/behavior changes.

### Flow Cytometry

Tumors were separated from the mammary gland and placed in six-well plates with 2 mL of Click's media without mercaptoethanol or L-glutamine (Irvine Scientific, Santa Ana, CA), where they were minced with scalpels, digested with 500 units/ml collagenase type II and IV and 20 μg/ml DNase (Worthington Biochemical Corporation, Lakewood, NJ) and incubated for 1h at 37° C. The tissue suspension was then filtered through a 100 μm strainer and washed with Click's. The filtered cells were centrifuged at 1,400 RPM for 5 min, the supernatant was removed, and the pellet was resuspended in 1 mL FACS buffer (500 mL 1×HBSS pH 7.4, 0.1% BSA, 0.02% sodium azide, up to 1 L ddH2O). The tumor cells were stained with BD viability 510 dye prior to staining with CD45 (clone30-F11), CD8a APC/Cy7 (clone 53-6.7) (1:400), CD4 APC or PerCp-Cy5.5 (clone RM4-5) (1:300), CD8 T cells were identified from live, CD3+/CD8+ Cells were run on the DakoCytomation CyAn ADP flow cytometer (Fort Collins, CO) or FACs Canto II, acquired using Summit software or Diva Software, and analyzed with FlowJo software (Tree Star, Ashland, OR). Geometric mean fluorescence intensity (gMFI) was calculated with FlowJo software. Cells were isolated from the tissue and treated with or without (unstimulated controls) phorbol 12-myristate 13-acetate (PMA) (20 ng/ml) (Sigma, St. Louis, MO) plus ionomycin (1 μg/ml) (Sigma, St. Louis, MO) for 4-6 h at 37 degrees in the presence of 2 μg/ml of brefeldin A (Adipogen, San Diego, CO) in RPMI+2.5% FBS. Cells were then stained with CD8, CD45, CD4, Gating was determined based on unstimulated controls. All antibodies were purchased from Biolegend (San Diego, CA).

### Data Mining Assays

Kaplan-Meir plotter was used to assess relapse-free survival (RFS) data for 5,143 breast cancer patients with a mean follow-up of 69 months. SEMA7A was queried and stratified into high and low expression groups using the autoselect best cutoff level function. The generated data were exported, graphed, and analyzed in GraphPad Prism to calculate hazard ratio (HR) with 95% confidence intervals and log-rank P values. The Gene Expression-Based Outcome for Breast Cancer Online platform (http://co.bmc.lu.se/gobo/) was used to query SEMA7A expression in 1,881 available sample tumors and a panel of 51 breast cancer cell lines. Data were stratified as high and low SEMA7A based on median expression and reported by molecular subtype. Outcome data were reported only in patients with ER+ breast cancer. The Oncomine Platform (https://www.oncomine.org) was used to query SEMA7A in available breast cancer data sets. The P value threshold was set to 0.05. All fold changes and gene ranks were accepted. Data are reported from the molecular subtype analysis, correlation with stage and grade, and drug-sensitivity screens.

### Cell Culture

MCF10A/MCF12A cells were obtained from, and cultured, according to ATCC. MCF10DCIS.com cells were obtained. Cells were validated by the DNA sequencing and found to be pure populations of their respective cell lines. Cells were regularly tested for mycoplasma throughout the studies. Cells were sub-cultured as previously described (reference), or according to ATCC standards. Exogenous SEMA7A protein utilized in our studies was purified from media of cells stably transfected with an Fc-tagged version of SEMA7A by published methods. Control plasmid (pcDNA3.1) was obtained.

shRNA Knockdown and Overexpression

shRNA plasmids targeting Sema7a, and a negative control shRNA (SABiosciences), were amplified in E. coli and plasmid DNA was isolated by Plasmid Maxi-Prep (Qiagen). MCF10DCIS.com-GFP cells were cultured overnight to ˜80% confluence. 1 μg/uL of each shRNA was added to Transfectagro Reduced-Serum Medium (Corning) and incubated for 15 min with 4 uL of X-treme Gene HP DNA transfection reagent (Roche). Transfected cells were selected for hygromycin resistance. Stable knockdown was confirmed by qPCR as shown in Black et al [2]. Negative control cells were transfected with a scrambled artificial sequence not matching human, mouse, or rat. Overexpression plasmid (SEMA7A-Fc) was a generous gift from R. Medzhitov (Yale University, New Haven, CT). All other over-expression plasmids (p304-V5-Blasticidin and V5-SEMA7A) were obtained from the Functional Genomics Core at the CU Anschutz Medical Campus and overexpression was confirmed via qPCR and western blot analysis. KD and overexpression as previously described.

### Animal Studies

All animal procedures were approved by the University of Colorado Anschutz Medical Campus Institutional Animal Care and Use Committee. Sema7atm1Alk/J mice (a generous gift from Alex Kolodkin at John's Hopkins University) and C57Bl/6 (Jax) were housed and bred as previously described [28]. Briefly, C57Bl/6 SEMA7Atm1Alk females were bred with WT C57Bl/6 males to induce pregnancy, lactation, and postpartum involution. Pup numbers were normalized to 6-8 per dam after birth to ensure for adequate lactation. Postpartum involution was initiated by forced weaning of pups at the peak of lactation (L10-L12). #4 right and left mammary glands were harvested from age-matched nulliparous animals and postpartum dams at involution days 1,2,4,6,8,10,14, and 28. For flow cytometry, inguinal lymph nodes were removed prior to tissue digestion. #3 mammary glands were also harvested, formalin-fixed, paraffin-embedded, and 5 μm sections generated for histologic/immunohistochemical analysis. See Supplemental Table 1 for animal study sample sizes.

### In Vitro Survival Assays

MCF10A and MCF12A cells were cultured with 200 ng/ml purified SEMA7A or PBS vehicle control in adherent (tissue culture plastic) or forced suspension conditions (ultra-low attachment plates (Corning, Corning, NY, USA: #3473)). Cells were seeded at a density of 1000 cells/well in a 96-well plate plus media only wells as controls. Cell death was measured 24 h post seeding via luminescence using the Caspase Glo assay (Promega, Madison, WI, USA: #G8090). Annexin-V/7AAD staining (Biolegend, San Diego, CA, USA: #640930) and pAKTS473 (ThermoFisher: Waltham, MA, USA, #17-9715-42) were also used to confirm cell viability by flow cytometry. See flow cytometry methods for staining protocol. Function blocking antibodies for b1 (CD29−9EG7; BD Biosciences: #550531) and α6 integrin (ThermoFisher: #14-0495-82) were used to disrupt integrin signaling in the presence of SEMA7A or as antibody alone controls to determine off-target effects. IgG controls were also used. 9EG7 was used at a concentration of 0.6 μg/ml while GoH3 was used at a concentration of 100 μg/mL for inhibition studies consistent with previous reports [29, 30]. Cells were cultured with anti-integrin inhibitors at time of seeding.

### Label Retention Stem Cell Assay

MCF10A and MCF12A cells were labeled with CellTrace Violet (ThermoFisher: #C34557) for 20 min at 37° ° C. according to the manufacturer's instructions. A sample of labeled cells was analyzed by flow cytometry to determine labeling efficiency and fluorescent intensity at time of seeding (Supplemental FIG. 1) Single cells were seeded at a density of 4000 cells/well in ultra-low attachment 24-well plates to induce mammosphere formation. Cells were cultured in normal culture media for 7 days and were subsequently counted, dissociated enzymatically in Accutase, (Stem Cell Technologies, Vancouver, Canada: #07922), and analyzed by flow cytometry. See flow cytometry methods for detailed information.

### Paclitaxel Resistance Studies

MCF10DCIS.com cells were treated with 100 nM paclitaxel (Millipore Sigma, Burling, MA, USA #T7402-5MG) for 24 h prior to analysis by flow cytometry. Dose was determined experimentally based on cell viability and cancer stem cell enrichment. Drug resistance was measured by label-retention and mammosphere formation as described above and % CD44+(Biolegend: #103018) CD24− (Biolegend: #311104) of singlets was used as a molecular definition of drug-resistant stem cells. See label retention stem cell assay and flow cytometry methods for additional details.

### Flow Cytometry

Single cell suspensions were generated from cultured cells via enzymatic harvest with Accutase. Mouse mammary tissues were minced with scalpels, digested in Click's media containing 500 units/mL Collagenase II (Worthington, Columbus, OH, USA; LS004174) and Collagenase IV (Worthington, LS004186) and 20 μg/mL DNAse (Worthington, LS002004) for 1 h at 37° C. with occasional trituration and strained through 70 μm filters to generate single cell suspensions. Single cells were stained at 4° C. for 30 min and washed with PBS+2% FBS. Cells were filtered through 30 μm filters, diluted to <106 cells/ml, and analyzed with the ZE5-YETI flow cytometer. Fixation/Permeabilization kit (BD Biosciences, Franklin Lakes, NJ, USA: #554715) was used for intracellular antigen staining. Data were analyzed with Kaluza software. Single stain, FMO, and unstained controls were used to set gates. Single cells were lineage depleted using CD45− (BD Biosciences: #564279)/CD31− (BD Biosciences: #745436) gating. Full gating strategy is included as Supplemental FIG. 2.

### Immunohistochemistry

Tissues were formalin-fixed and paraffin-embedded as previously described [31]. Hematoxylin and Eosin staining was used to define morphological features. Antigen retrieval was performed using target retrieval solution (Dako, Glostrup, Denmark; #S1699) for CC3 (Cell Signaling Technologies, Danvers, MA, USA; #9661) and Perilipin (Cell Signaling: #3470) or (EDTA Dako cat #S2367) for pSTAT3 (Cell Signaling Technologies; #9145). ImmPRESS polymer anti-rabbit IgG secondary reagent (Vector Labs, San Francisco, CA, USA t #MP-7401-15) was used for secondary staining of CC3 and pSTAT3 stained tissues and anti-rabbit secondary (Dako #K4003) for perilipin stains. DAB (Vector Labs; #SK-4105) and counterstaining were performed with hematoxylin (Vector Labs #H-3401). Alveolar area was measured on H&E-stained tissues in ImageJ on 10 alveoli/field; 5 fields/tissue. 10 fields/tissue were analyzed for positive CC3, pSTAT3, and perilipin stained tissues using CellSense Dimension software count and measure feature on regions of interest.

### Data Mining

Analysis of SEMA7A in breast cancer versus normal was performed on http://ualcan.path.uab.edu/index.html by selecting breast cancer samples from The Cancer Genome Atlas (TCGA). Five-year relapse-free survival (RFS) curves for Sema7a (Affy ID: 230345_at) and Itga6 (Affy ID: 201656_at) were generated in KM plot using default parameters and no restrictions. Patients split by median survival. For multi-gene analyses, genes were filtered by median expression.

### Statistical Analysis

Unpaired t-test and one-way ANOVA were performed in GraphPad Prism. Nonparametric equivalent analyses were used for samples with uneven distribution or unequal standard deviations. p values of <0.05 were considered significant. Error bars represent mean±standard error of the mean. All in vivo studies were replicated in at least three mice and in vitro studies performed in technical and biological triplicate with representative data presented.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as can be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A synthetic molecule comprising, an isolated or synthetic polypeptide comprising at least 75% homology to the polypeptide represented by SEQ. ID. NO:2, wherein the isolated or synthetic polypeptide fragment comprising at least 75% homology to the polypeptide represented by SEQ. ID. NO:2 is not part of a full-length naturally occurring Semaphorin 7a (SEMA7A) molecule.

2. The synthetic molecule according to claim 1, wherein the isolated or synthetic polypeptide is fused to a second molecule for formation of a fusion polypeptide.

3. The synthetic molecule according to claim 2, wherein the second molecule comprises an antibody fragment, an immunogen, a peptide transduction domain, or a conjugate.

4-6. (canceled)

7. An antibody or antibody fragment thereof against the isolated or synthetic polypeptide according to claim 1, wherein the antibody or antibody fragment thereof is capable of binding an isolated or synthetic polypeptide according to claim 1 and capable of at least one of inhibiting expression of SEMA7A, inhibiting activity of SEMA7A, and killing cells expressing SEMA7A.

8. The antibody according to claim 7, wherein the antibody comprises a monoclonal antibody or a polyclonal antibody.

9. (canceled)

10. A pharmaceutical composition comprising the antibody according to claim 7, and a pharmaceutically acceptable excipient.

11. The pharmaceutical composition according to claim 10, further comprising one or more anti-cancer agents or immunostimulatory agents.

12. The pharmaceutical composition according to claim 10, wherein the one or more anti-cancer agents or immunostimulatory agents comprise one or more of an anti-BCL-2, anti-PD-L1, or anti-PD-1 antibody.

13. (canceled)

14. The pharmaceutical composition according to claim 10, wherein pharmaceutical composition further comprises an anti-estrogen therapeutic, an estrogen receptor antagonist, or other endocrine modulating therapeutic.

15-16. (canceled)

17. A polynucleotide encoding the polypeptide according to claim 1.

18. The polynucleotide according to claim 17, further comprising a vector for expressing the encoded polypeptide.

19. A host cell comprising the polynucleotide according to claim 17.

20. A method for treating a subject having a health condition with elevated SEMA7A expression, comprising administering to the subject having the health condition, a pharmaceutical composition according to claim 10 and treating the health condition.

21. The method according to claim 20, wherein the health condition comprises cancer.

22. (canceled)

23. The method according to claim 21, wherein the cancer comprises breast cancer.

24. (canceled)

25. The method according to claim 23, wherein the breast cancer comprises estrogen receptor positive or estrogen receptor negative breast cancer (ER+BC, ER−BC) or postpartum breast cancer (PPBC).

26. (canceled)

27. The method according to claim 20, wherein administering at least one additional therapeutic composition to the subject comprises administering one or more cell survival pathway inhibitor-containing composition, wherein the at least one additional therapeutic composition does not include an anti-estrogen-based therapy, does not include an anti-CDK4/6 therapy or does not include chemotherapy.

28. (canceled)

29. A method for predicting breast cancer relapse in a subject comprising obtaining at least one sample from a subject having breast cancer and analyzing the at least one sample for SEMA7A presence and/or concentration and predicting breast cancer relapse in the subject based on presence and/or concentration of SEMA7A.

30-32. (canceled)

33. A kit comprising the pharmaceutical composition according to claim 10; and at least one container.

34. A method for diagnosing risk for and prognosis of a health condition comprising, obtaining one or more blood samples from a subject, measuring concentration of SEMA7A in the blood sample, and treating the subject according to the level of SEMA7A in the blood sample.

35-36. (canceled)

37. The method according to claim 34, wherein the concentration of SEMA7A in the one or more blood sample indicative of the need for therapeutic intervention comprises about 0.1 ng/ml, or about 0.5 ng/ml to about 1.5 ng/ml or more.

38. A method for reducing the risk of onset or treating cancer in a subject expressing or having elevated concentrations of SEMA7A comprising administering a pharmaceutical composition according to claim 10 to the subject.

39. A method for reducing or eliminating at least one of cancer stem cells, cancer stem cell activities and cancer cell metastasis comprising, treating a subject having cancer with a pharmaceutical composition capable of inhibiting at least one of SEMA7A expression and SEMA7A activity; and reducing or eliminating at least one of cancer stem cells, cancer stem cell activities and cancer cell metastasis.

40-42. (canceled)
